SEQUENCE LISTING (2 sequences)

SEQ ID NO: 1 - protein, 120 aa, synthetic construct
  REGION 1..120: note = Synthetic Construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 2 - protein, 113 aa, synthetic construct
  REGION 1..113: note = Synthetic Construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKR

(19) United States
(12) Patent Application Publication
(10) Pub. No.: US 2023/0330241 A1
(43) Pub. Date: Oct. 19, 2023

(54) HUMANIZED ANTI-LIV1 ANTIBODIES FOR THE TREATMENT OF CANCER

(71) Applicant: Seagen Inc., Bothell, WA (US)

(72) Inventors: Emily STEVENS, Bothell, WA (US); Hong LI, Bothell, WA (US); William HANLEY, Bothell, WA (US); Phillip GARFIN, Bothell, WA (US); Shawna HENGEL, Bothell, WA (US)

(21) Appl. No.: 18/171,208

(22) Filed: Feb. 17, 2023

Related U.S. Application Data
(63) Continuation of application No. 16/934,745, filed on Jul. 21, 2020, now abandoned.
(60) Provisional application No. 63/031,496, filed on May 28, 2020.
(60) Provisional application No. 63/003,613, filed on Apr. 1, 2020.
(60) Provisional application No. 62/890,498, filed on Aug. 22, 2019.
(60) Provisional application No. 62/877,233, filed on Jul. 22, 2019.

(51) Int. Cl. A61K 47/68; A61P 35/00; A61K 9/00; C07K 16/28

(52) U.S. Cl. CPC A61K 47/6801; A61P 35/00; A61K 9/0019; C07K 16/2818; C07K 16/2827

(57) ABSTRACT

Methods for using anti-LIV1 antibodies and antibody-drug conjugates, including anti-LIV1 antibody-drug conjugates, to inhibit proliferation of a cell, such as a LIV1-expressing cell, as well as for the treatment of cancers, such as, e.g., LIV1-associated solid tumors and breast cancer (e.g., locally advanced or metastatic breast cancer), are provided.

### CROSS-REFERENCE TO RELATED APPLICATIONS

This application is a Continuation application of U.S. Ser. No. 16/934,745, filed on Jul. 21, 2020, which claims priority to U.S. Provisional Application No. 62/877,233 filed Jul. 22, 2019, U.S. Provisional Application No. 62/890,498 filed Aug. 22, 2019, U.S. Provisional Application No. 63/003,613 filed Apr. 1, 2020, and U.S. Provisional Application No. 63/031,496 filed May 28, 2020 the contents of each of which are incorporated herein by reference in their entirety.

### REFERENCE TO AN ELECTRONIC SEQUENCE LISTING

The contents of the electronic sequence listing (761682002101SEQLIST.xml; Size: 3,766 bytes; and Date of Creation: Feb. 8, 2023 (is herein incorporated by reference in its entirety.

### FIELD OF THE INVENTION

The present invention relates to the field of antibody-based cancer therapeutics. In particular, the present invention relates to the use of humanized anti-LIV1 antibodies and antigen-binding fragments or antibody-drug conjugates thereof (e.g., LIV1-antibody-drug conjugates (LIV1-ADCs) for the treatment of cancer, such as solid tumors, such as, e.g., locally advanced or metastatic solid tumors (e.g., small cell lung cancer, non-small cell lung cancer, head and neck squamous cell carcinoma, esophageal squamous cell carcinoma, and gastric and gastroesophageal junction adenocarcinoma), and breast cancer (e.g., locally advanced or metastatic breast cancer).

### BACKGROUND

LIV1 (SLC39A6) is a member of the solute carrier family, a multi-span transmembrane protein with putative zinc transporter and metalloproteinase activity. LIV1 was first identified as an estrogen-induced gene in the breast cancer cell line ZR-75-1. LIV-1 is expressed in most subtypes of metastatic breast cancer.

Cancer remains to be one of the most deadly threats to human health. In the U.S., cancer affects nearly 1.3 million new patients each year, and is the second leading cause of death after heart disease, accounting for approximately 1 in 4 deaths. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Lung cancer remains the leading cause of death from cancer in the United States, with over 155,000 deaths estimated in 2017. Treatments with curative intent for patients with early stage disease include surgery, chemotherapy, radiation therapy, or a combined modality approach. However, a majority of patients are diagnosed with advanced stage disease, which is usually incurable. Non-small cell lung cancer (NSCLC) represents up to 80% of all lung cancers. Within the subtypes of NSCLC, squamous cell carcinoma (SCC/NSCLC) represents approximately 30% of NSCLC. Systemic therapies used in the metastatic setting for SCC/NSCLC have shown limited benefit and are primarily aimed at prolonging survival and maintaining the quality of life for as long as possible, while minimizing side effects due to treatment. First line treatment for patients with SCC/NSCLC whose tumors do not express high levels of PD-L1 include a platinum-based chemotherapy doublet that does not contain pemetrexed, anti-VEGF antibody, or an anti-EGFR antibody necitumumab in combination with gemcitabine and cisplatin. Patients with at least 50% tumor cell staining for PD-L1 are offered first-line treatment with the anti-PD-1 inhibitor pembrolizumab. Patients who progress on an initial combination chemotherapy regimen may receive an anti-PD-1 or PD-L1 antibody, and combination chemotherapy is considered for patients whose disease has progressed after receiving PD-1/L1 inhibitors. New classes of therapy are urgently needed that can provide meaningful benefit to SCC/NSCLC patients.

Head and neck cancers make up approximately 3% of cancers in the United States. Over 63,000 cases are estimated to have been diagnosed in 2017 and more than 13,000 patients died from this disease. Though human papilloma virus (HPV) infection also appears to contribute to head and neck cancers. More than 90-95% of oral and nasopharyngeal cancers are of squamous histology. Surgical resection, radiotherapy, and/or chemoradiation are frequently recommended for patients with early-stage or localized disease. Palliative chemotherapy, immunotherapy and/or supportive care are the most appropriate options for patients with locally recurrent or metastatic disease that are not amenable to definitive therapy. Platinum-based regimens are the preferred standard of care treatment for patients with recurrent or de novo metastatic squamous cell carcinoma of the head and neck (SCCHN). Cetuximab in combination with a platinum/5-FU regimen has demonstrated clinically meaningful benefits compared to platinum/5-FU alone. For patients progressing on first line treatment, second line treatment is with single agent chemotherapy, targeted therapy, or a checkpoint inhibitor such as nivolumab or pembrolizumab. Overall, there is a great unmet medical need for patients with SCCHN that have progressed after first line platinum combination therapy followed by second line PD-1 therapy.

Esophageal cancer is the sixth leading cause of cancer-related mortality worldwide due to its overall poor prognosis. The global age-standardized incidence rate of esophageal squamous cell carcinoma (ESCC) is 1.4-13.6 per 100,000 people. Esophageal cancer is estimated to be responsible for 15,690 deaths and 16,940 new cases in the United States in 2016. The majority of patients present with locally advanced or systemic disease and outcomes remain poor despite advances in treatment. More effective treatments for these patients with locally advanced or systemic disease are urgently needed.

Gastric cancer, or stomach cancer, is most commonly caused by infection by the bacteria Helicobacter pylori. About 90 to 95% of cancers of the stomach are adenocarcinomas. Gastric cancer occurs mostly in adults (average age at diagnosis: 69 years). The incidence of gastric cancer is about 1 in 111. The overall 5-year relative survival rate of all people with gastric cancer in the United States is about 29%. Gastroesophageal junction adenocarcinoma is a cancer of the lower part of the esophagus. The incidence of gastroesophageal junction adenocarcinoma is rising rapidly in western countries, the treatment options are limited and the overall prognosis is extremely poor.

Breast cancers are classified on the basis of three protein expression markers: estrogen receptor (ER), progesterone receptor (PgR), and the overexpression of the growth factor receptor HER2/neu. Hormonal therapies, including tamoxifen and aromatase inhibitors, can be effective in treating tumors that express the hormone receptors ER and PgR. HER2-directed therapies are useful for tumors that express HER2/neu; these tumors are the only class of breast cancer that is currently eligible for immunotherapy. For these patients, unconjugated antibodies, such as Herceptin or Perjeta, are generally used in combination with chemotherapy.

There is clearly a significant need for effective treatments for solid tumors, particularly locally advanced or metastatic solid tumors, and breast cancer, particularly late-stage breast cancer. The present invention meets the need for improved treatment of solid tumors, such as, e.g., locally advanced or metastatic solid tumors (e.g., small cell lung cancer, non-small cell lung cancer, head and neck squamous cell carcinoma, esophageal squamous cell carcinoma, and gastric and gastroesophageal junction adenocarcinoma), and breast cancer by providing a highly specific and effective anti-LIV1-antibody-drug conjugate.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating a subject having or at risk of having a LIV1-associated cancer, comprising administering to the subject a therapeutically effective dose of a LIV1 antibody-drug conjugate (LIV1-ADC), wherein the LIV1-ADC comprises a humanized hLIV22 antibody conjugated to a vcMMAE (valine-citruline-monomethyl aurstating E), wherein the hLIV22 antibody comprises a heavy chain variable region comprising the sequence of SEQ ID NO:1 and a light chain variable region comprising the sequence of SEQ ID NO:2, wherein the vcMMAE has the structure:

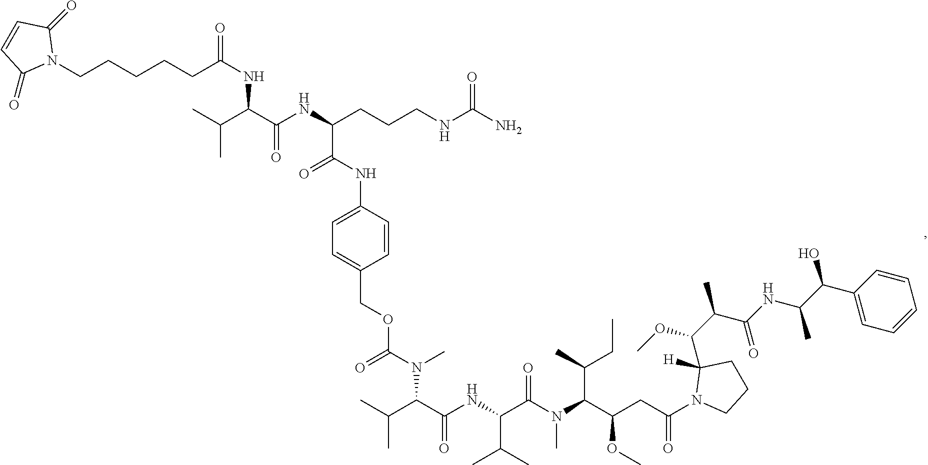 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

wherein the LIV1-ADC is administered about once per week. In some embodiments, the LIV1-ADC is administered at a dose of about 0.5 mg/kg to about 2 mg/kg of body weight of the subject. In some embodiments, the LIV1-ADC is administered at a dose of about 0.75 mg/kg to about 1.67 mg/kg of body weight of the subject. In some embodiments, the LIV1-ADC is administered at a dose of about 0.75 mg/kg of body weight of the subject. In some embodiments, the LIV1-ADC is administered at a dose of about 1.0 mg/kg of body weight of the subject. In some embodiments, the LIV1-ADC is administered at a dose of about 1.25 mg/kg of body weight of the subject. In some embodiments, the LIV1-ADC is administered at a dose of about 1. 5 mg/kg of body weight of the subject. In some embodiments, the LIV1-ADC is administered at a dose of about 1.75 mg/kg of body weight of the subject.

Also provided herein are methods of treating a subject having or at risk of having a LIV1-associated cancer, comprising administering to the subject a therapeutically effective dose of a LIV1 antibody-drug conjugate (LIV1-ADC), wherein the LIV1-ADC comprises a humanized hLIV22 antibody conjugated to a vcMMAE (valine-citruline-monomethyl aurstating E), wherein the hLIV22 antibody comprises a heavy chain variable region comprising the sequence of SEQ ID NO:1 and a light chain variable region comprising the sequence of SEQ ID NO:2, wherein the vcMMAE has the structure:

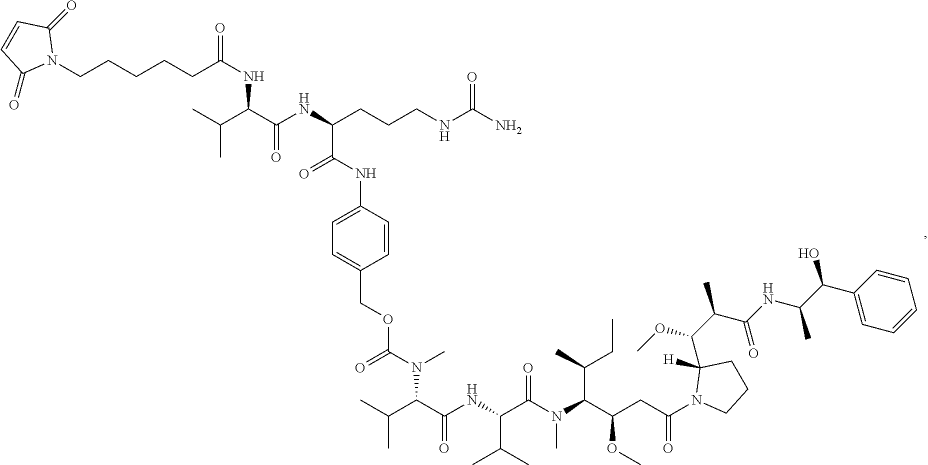 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

wherein the LIV1-ADC is administered twice in a three week treatment cycle. In some embodiments, the LIV1-ADC is administered on day 1 and day 8 of the three week treatment cycle. In some embodiments, the LIV1-ADC is administered is administered at a dose of about 0.5 mg/kg to about 3.0 mg/kg of body weight of the subject. In some embodiments, the LIV1-ADC is administered is administered at a dose of about 1.0 mg/kg to about 2.5 mg/kg of body weight of the subject. In some embodiments, the LIV1-ADC is administered is administered at a dose of about 1.25 mg/kg of body weight of the subject. In some embodiments, the LIV1-ADC is administered is administered at a dose of about 1.5 mg/kg of body weight of the subject. In some embodiments, the LIV1-ADC is administered is administered at a dose of about 1.75 mg/kg of body weight of the subject. In some embodiments, the LIV1-ADC is administered is administered at a dose of about 2.0 mg/kg of body weight of the subject. In some embodiments, the LIV1-ADC is administered is administered at a dose of about 2.5 mg/kg of body weight of the subject.

In some embodiments, a vcMMAE to hLIV22 ratio is from 1 to 8. In some embodiments, the average value of the vcMMAE to hLIV22 ratio in a population of the LIV1-ADC is about 4. In some embodiments, the LIV1-associated cancer is a breast cancer. In some embodiments, the breast cancer is estrogen receptor positive (ER+) breast cancer. In some embodiments, the breast cancer is progesterone receptor positive/human epidermal growth factor receptor 2 negative breast (PR+/HER2−) cancer. In some embodiments, the breast cancer is a triple negative breast cancer. In some embodiments, the breast cancer is hormone receptor positive (HR+) breast cancer. In some embodiments, the breast cancer is HER2 positive breast cancer. In some embodiments, the breast cancer is HR+/HER2 negative breast cancer. In some embodiments, the cancer is an advanced stage cancer. In some embodiments, the advanced stage cancer is a stage 3 or stage 4 cancer. In some embodiments, the cancer is metastatic cancer. In some embodiments, the cancer is unresectable. In some embodiments, the cancer is locally advanced. In some embodiments, the cancer is recurrent cancer. In some embodiments, the subject received prior treatment with standard of care therapy for the cancer and failed the prior treatment. In some embodiments, the subject has been previously treated with one or more therapeutic agents and did not respond to the treatment, wherein the one or more therapeutic agents is not the LIV1-ADC. In some embodiments, the subject has been previously treated with one or more therapeutic agents and relapsed after the treatment, wherein the one or more therapeutic agents is not the LIV1-ADC. In some embodiments, the subject has been previously treated with one or more therapeutic agents and has experienced disease progression during treatment, wherein the one or more therapeutic agents is not the LIV1-ADC. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express LIV1. In some embodiments, one or more therapeutic effects in the subject is improved after administration of the LIV1-ADC relative to a baseline. In some embodiments, the one or more therapeutic effects is selected from the group consisting of: size of a tumor derived from the cancer, objective response rate, duration of response, time to response, progression free survival, and overall survival. In some embodiments, the route of administration for the LIV1-ADC is intravenous infusion. In some embodiments, the LIV1-ADC is administered as a monotherapy. In some embodiments, the LIV1-ADC is administered in combination with trastuzumab. In some embodiments, the LIV1-ADC is in a pharmaceutical composition comprising the LIV1-ADC and a pharmaceutically acceptable carrier. In some embodiments, the subject is a human.

Also provided here are kits comprising
(a) a dosage ranging from about 0.5 mg/kg to about 3.0 mg/kg of a LIV1-ADC; and
(b) instructions for using the LIV1-ADC according to any of the methods provided herein.

Also provided herein are methods of treating a subject having or at risk of having a LIV1-associated cancer, comprising administering to the subject a therapeutically effective dose of an antibody or an antigen-binding fragment thereof that specifically binds human LIV1, wherein the antibody or antigen-binding fragment thereof comprises a heavy chain variable region (HCVR) having at least 95% identity to SEQ ID NO:1, and a light chain variable region (LCVR) having at least 95% identity to SEQ ID NO:2, wherein the cancer is a solid tumor. In some embodiments, the heavy chain variable region of the antibody or antigen-binding fragment thereof comprises the three complementarity determining regions (CDRs) of SEQ ID NO:1 and the light chain variable region of the antibody or antigen-binding fragment thereof comprises the three CDRs of SEQ ID NO:2. In some of the embodiments herein, the heavy chain variable region comprises the sequence of SEQ ID NO:1 and the light chain variable region comprises the sequence of SEQ ID NO:2. In some of the embodiments herein, the antibody or antigen-binding fragment thereof is conjugated to monomethyl auristatin E (MMAE):

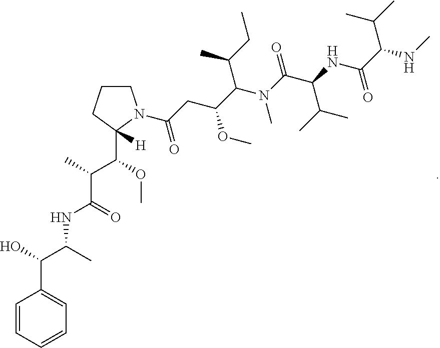 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In some of the embodiments herein, the antibody or antigen-binding fragment thereof is conjugated to valine-citrulline-monomethyl auristatin E (vcMMAE):

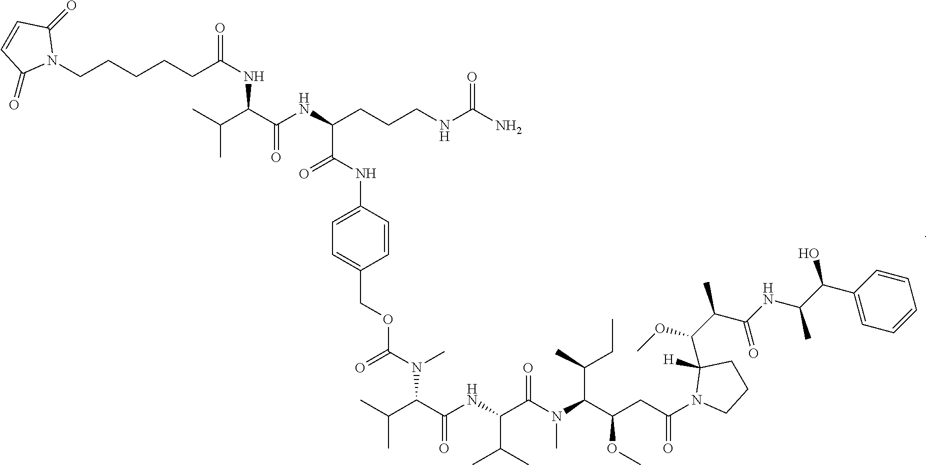 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some of the embodiments herein, the dose is about 2.5 mg/kg of body weight of the subject. In some of the embodiments herein, the dose administered is less than about 200 mg of the antibody or antigen-binding fragment thereof per treatment cycle. In some of the embodiments herein, the dose administered is less than about 250 mg of the antibody or antigen-binding fragment thereof per treatment cycle. In some of the embodiments herein, the treatment cycle is a Q3W treatment cycle. In some of the embodiments herein, the dose is about 1.0 mg/kg of body weight of the subject. In some of the embodiments herein, the dose administered is less than about 100 mg of the antibody or antigen-binding fragment thereof per treatment cycle. In some of the embodiments herein, the dose is about 1.25 mg/kg of body weight of the subject. In some of the embodiments herein, the dose administered is less than about 125 mg of the antibody or antigen-binding fragment thereof per treatment cycle. In some of the embodiments herein, the treatment cycle is a Q1W treatment cycle. In some of the embodiments herein, the subject has been previously treated with one or more therapeutic agents and did not respond to the treatment, wherein the one or more therapeutic agents is not the antibody or antigen-binding fragment thereof. In some of the embodiments herein, the subject has been previously treated with one or more therapeutic agents and relapsed after the treatment, wherein the one or more therapeutic agents is not the antibody or antigen-binding fragment thereof. In some of the embodiments herein, the subject has been previously treated with one or more therapeutic agents and has experienced disease progression during treatment, wherein the one or more therapeutic agents is not the antibody or antigen-binding fragment thereof. In some of the embodiments herein, the solid tumor is selected from the group consisting of lung cancer, head and neck cancer, esophageal cancer, gastric cancer, and gastroesophageal junction cancer. In some of the embodiments herein, the solid tumor is lung cancer. In some of the embodiments herein, the lung cancer is small cell lung cancer. In some of the embodiments herein, the lung cancer is non-small cell lung cancer. In some of the embodiments herein, the non-small cell lung cancer is non-squamous cell carcinoma. In some of the embodiments herein, the non-small cell lung cancer is squamous cell carcinoma. In some of the embodiments herein, the solid tumor is head and neck cancer. In some of the embodiments herein, the head and neck cancer is squamous cell carcinoma. In some of the embodiments herein, the solid tumor is esophageal carcinoma. In some of the embodiments herein, the esophageal carcinoma is squamous cell carcinoma. In some of the embodiments herein, the solid tumor is gastric cancer. In some of the embodiments herein, the gastric cancer is gastric adenocarcinoma. In some of the embodiments herein, the solid tumor is gastroesophageal junction cancer. In some of the embodiments herein, the gastroesophageal junction cancer is gastroesophageal junction adenocarcinoma. In some of the embodiments herein, the cancer is an advanced stage cancer. In some of the embodiments herein, the advanced stage cancer is a stage 3 or stage 4 cancer. In some of the embodiments herein, the advanced stage cancer is metastatic cancer. In some of the embodiments herein, the cancer is recurrent cancer. In some of the embodiments herein, the cancer is unresectable. In some of the embodiments herein, the subject received prior treatment with standard of care therapy for the cancer and failed the prior treatment. In some of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express LIV1. In some of the embodiments herein, one or more therapeutic effects in the subject is improved after administration of the antibody or antigen-binding fragment thereof relative to a baseline. In some of the embodiments herein, the one or more therapeutic effects is selected from the group consisting of: size of a tumor derived from the cancer, objective response rate, duration of response, time to response, progression free survival, and overall survival. In some of the embodiments herein, the route of administration for the antibody or antigen-binding fragment thereof is intravenous infusion. In some of the embodiments herein, the antibody or antigen-binding fragment thereof is administered as a monotherapy. In some of the embodiments herein, the antibody or antigen-binding fragment thereof is administered in combination with a checkpoint inhibitor. In some of the embodiments herein, the checkpoint inhibitor is an anti-PD-1 antibody, an anti-PD-L1 antibody, an anti-CTLA4 antibody, B7-DC-Fc, LAG3, or TIM3. In some of the embodiments herein, the checkpoint inhibitor is selected from the group consisting of MEDI0680, AMP-224, nivolumab, pembrolizumab, pidilizumab, MEDI4736, MPDL3280A, ipilimumab and tremelimumab. In some of the embodiments herein, the checkpoint inhibitor is pembrolizumab. In some of the embodiments herein, the antibody or antigen-binding fragment thereof is in a pharmaceutical composition comprising the antibody or antigen-binding fragment thereof and a pharmaceutically acceptable carrier. In some of the embodiments herein, the subject is a human.

Also provided herein are kits comprising:
(a) a dosage ranging from about 0.5 mg/kg to about 2.8 mg/kg of an antibody or antigen-binding fragment thereof that binds LIV1; and
(b) instructions for using the antibody or antigen-binding fragment thereof according to some of the methods provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic displaying the dose finding (upper panel) and expansion (lower panel) protocol, where 1.25 mg/kg on a D1,8-Q3wk schedule was selected as the starting dose level. The total exposure per cycle (mg/kg/cycle) and the approximate relative exposure (x AUC) compared to the starting dose are also displayed.

FIG. 2 is a table displaying total dose and relative exposure (relative AUC) under a weekly dosing schedule (Q1W) or a D1,8-Q3W dosing schedule at different dose levels.

FIG. 3A is a scatter plot showing the change of drug-antibody ratio (DAR) in blood ADC for subjects over 7 days after LV administration in Q3W dosing. FIG. 3B-C are graphs showing the pharmacokinetics modeling of ADC with DAR ≥4 and ADC with DAR=2-3 over time respectively for LV administration at 3 mg/kg on Q3wk schedule (red line) or at 1.0 mg/kg on Q1wk schedule (black line).

FIG. 4A-H is a series of graphs showing the pharmacokinetics modeling of LV, wherein the respective concentrations of ADC with DAR ≥4, ADC with DAR=2-3, ADC with DAR=1, ADC with DAR=0, total antibody, ADC, antibody-conjugated MMAE and free MMAE over time were simulated for LV administration at 1.5 mg/kg on a D1,8-Q3wk schedule (red line) or at 1.0 mg/kg on a Q1wk schedule (black line). FIG. 4I is a summary table showing the simulated exposure measured by area-under-curve for the first 21 days post-administration (AUC₀₋₂₁day), and C_{trough} values for the respective species subsequent to LV administration under either a D1,8-Q3wk or a Q1wk dosing scheme.

FIG. 5A-E is a series of graphs showing pharmacokinetics modeling of BV (red line) and LV (blue line), wherein the measured total ADC concentration over time (FIG. 5A), and the respective simulated concentrations of ADC with DAR ≥4, ADC with DAR=2-3, ADC with DAR=1, and ADC with DAR=0 over time were displayed (FIG. 5B-5E).

FIG. 6A-C is a series of graph showing the correlation of treatment efficacy with pharmacokinetics of LV, where the probability of response was plotted against the C_{trough} value of ADC (FIG. 6A), Cₘₐₓ value of ADC (FIG. 6B) and C_{trough} value of MMAE (FIG. 6C).

FIG. 7A is a table summarizing the probability of peripheral neuropathy (NP), NP of Grade 2 or higher (Gr≥2 PN) or neutropenia at the indicated standardized dose levels (mg/kg/wk) when subjects were administered with BV under a Q3W or a Q1W dosing schedule. The upper panel of FIG. 7B shows a plot of probability of Gr≥2 PN versus the standardized dose received before such Gr≥2 PN was observed for a Q3W dosing schedule (blue) or a Q1W dosing schedule (red), while the lower panel of FIG. 7B is a survival regression analysis showing the Hazard Ratio (HR), and the 95% confidence interval (95% CI) of Q1W vs. Q3W.

FIG. 8A-B are graphs showing the pharmacokinetics modeling of ADC and MMAE concentrations over time when LV was administered at 2.5 mg/kg D1Q3wk (black line); 0.75 mg/kg QW (blue line); 1 mg/kg D1,8,15-Q4wk (red line); or 1.25 mg/kg D1,8-Q3wk (green line). FIG. 8C is a summary table displaying the respective peak to trough fluctuation (Cₘₐₓ/C_{trough}) and the total exposure (AUCₜₐᵤ) for ADC and MMAE.

FIG. 9A-B are graphs showing the pharmacokinetics modeling of ADC and MMAE concentrations over time when LV was administered under Q3wk at 2.5 mg/kg (black line); or under D1,8-Q3wk at either 1.0 mg/kg (blue line), 1.25 mg/kg (red line), 1.5 mg/kg (green line), or 1.75 mg/kg (purple line). FIG. 9C is a summary table displaying the respective peak to trough fluctuation (Cₘₐₓ/C_{trough}) and the total exposure (AUCₜₐᵤ) for ADC and MMAE.

FIG. 10A-B are graphs showing the pharmacokinetics modeling of ADC and MMAE concentrations over time when LV was administered under Q3W at 2.5 mg/kg; or under Q1W at either 1.0 mg/kg or 1.25 mg/kg.

FIG. 11 is a chart showing the dose escalation and dose expansion scheme for the phase 1 study described in Example 19.

### DETAILED DESCRIPTION

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

### I. Definitions

As used herein, including the appended claims, the singular forms of words such as “a,” “an,” and “the,” include their corresponding plural references unless the context clearly dictates otherwise.

An “antibody-drug conjugate” or “ADC” refers to an antibody conjugated to a cytotoxic agent or cytostatic agent. Typically, antibody-drug conjugates bind to a target antigen (e.g., LIV1) on a cell surface, followed by internalization of the antibody-drug conjugate into the cell and subsequent release of the drug into the cell. In certain exemplary embodiments, an antibody-drug conjugate is a LIV1-ADC.

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures. Substituents such as carbohydrate groups are generally not specified, but may be present nonetheless.

The terms “amino-terminal” and “carboxy-terminal” denote positions within polypeptides. Where the context allows, these terms are used with reference to a particular sequence or portion of a polypeptide to denote proximity or relative position. For example, a certain sequence positioned carboxy-terminal to a reference sequence within a polypeptide is located proximal to the carboxy terminus of the reference sequence, but is not necessarily at the carboxy terminus of the complete polypeptide.

For purposes of classifying amino acids substitutions as conservative or nonconservative, the following amino acid substitutions are considered conservative substitutions: serine substituted by threonine, alanine, or asparagine; threonine substituted by proline or serine; asparagine substituted by aspartic acid, histidine, or serine; aspartic acid substituted by glutamic acid or asparagine; glutamic acid substituted by glutamine, lysine, or aspartic acid; glutamine substituted by arginine, lysine, or glutamic acid; histidine substituted by tyrosine or asparagine; arginine substituted by lysine or glutamine; methionine substituted by isoleucine, leucine or valine; isoleucine substituted by leucine, valine, or methionine; leucine substituted by valine, isoleucine, or methionine; phenylalanine substituted by tyrosine or tryptophan; tyrosine substituted by tryptophan, histidine, or phenylalanine; proline substituted by threonine; alanine substituted by serine; lysine substituted by glutamic acid, glutamine, or arginine; valine substituted by methionine, isoleucine, or leucine; and tryptophan substituted by phenylalanine or tyrosine. Conservative substitutions can also mean substitutions between amino acids in the same class. Classes are as follows: Group I (hydrophobic side chains): Met, Ala, Val, Leu, Ile; Group II (neutral hydrophilic side chains): Cys, Ser, Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wisconsin). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least about 80%, at least about 85%, at about least 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

Designation of a range of values includes all integers within or defining the range.

In antibodies or other proteins described herein, reference to amino acid residues corresponding to those specified by SEQ ID NO includes post-translational modifications of such residues.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a cytostatic or cytotoxic drug.

The term “genetically engineered antibodies” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, e.g., complement binding and other effector functions. Typically, changes in the variable region are made to improve antigen-binding characteristics, improve variable region stability, and/or reduce the risk of immunogenicity.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12: 131-140, 1999; Nguyen et al., EMBO J. 19:921-930, 2000) or from VH domains of other species to produce single-domain antibodies (“dAbs,” see Ward et al., Nature 341: 544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CH1, hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1: 11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22: 195-208, 1985; Kindsvogel et al., DNA 1:335-343, 1982; Breiner et al., Gene 18: 165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31: 169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, M I D, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, M I D, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is substantially from a corresponding CDR in a non-human antibody when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Antibodies are typically provided in isolated form. This means that an antibody is typically at least about 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least about 60%, about 70%, about 80%, about 90%, about 95% or about 99% w/w pure of interfering proteins and contaminants from production or purification. Antibodies, including isolated antibodies, can be conjugated to cytotoxic agents and provided as antibody drug conjugates.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the LIV1-targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4th ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6th ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity. In certain exemplary embodiments, an anti-LIV1 IgG1 antibody of the invention mediates equal or increased ADCC relative to a parental antibody and/or relative to an anti-LIV1 IgG3 antibody.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig. In certain exemplary embodiments, an anti-LIV1 IgG1 antibody of the invention mediates equal or increased ADCP relative to a parental antibody and/or relative to an anti-LIV1 IgG3 antibody.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

The term “patient” or “subject” includes human and other mammalian subjects such as non-human primates, rabbits, rats, mice, and the like and transgenic species thereof, that receive either prophylactic or therapeutic treatment.

The term “effective amount,” in the context of treatment of a LIV1-expressing disorder by administration of an anti-LIV1 antibody or antigen-binding fragment thereof (e.g., a LIV1-ADC) as described herein, refers to an amount of such antibody or antigen-binding fragment thereof that is sufficient to inhibit the occurrence or ameliorate one or more symptoms of a LIV1-related disorder (e.g., a LIV1-expressing cancer). An effective amount of an antibody is administered in an “effective regimen.” The term “effective regimen” refers to a combination of amount of the antibody being administered and dosage frequency adequate to accomplish prophylactic or therapeutic treatment of the disorder (e.g., prophylactic or therapeutic treatment of a LIV1-expressing cancer).

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an anti-LIV1 antibody (e.g., a LIV1-ADC) is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

A “platinum-based therapy” refers to treatment with a platinum-based agent. A “platinum-based agent” refers to a molecule or a composition comprising a molecule containing a coordination complex comprising the chemical element platinum and useful as a chemotherapy drug. Platinum-based agents generally act by inhibiting DNA synthesis and some have alkylating activity. Platinum-based agents encompass those that are currently being used as part of a chemotherapy regimen, those that are currently in development, and those that may be developed in the future.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

### II. Anti-LIV1 Antibodies, Antigen-Binding Fragments and Antibody-Drug Conjugates

The present invention provides isolated, recombinant and/or synthetic human, primate, rodent, mammalian, chimeric, humanized and/or CDR-grafted antibodies and antigen-binding fragments and antibody-drug conjugates (e.g., a LIV1-ADC) thereof, as well as compositions and nucleic acid molecules comprising at least one polynucleotide encoding at least a portion of one antibody molecule. The present invention further includes, but is not limited to, methods of making and using such nucleic acids and antibodies including diagnostic and therapeutic compositions, methods and devices. In certain exemplary embodiments, humanized anti-LIV1 IgG1 antibodies are provided. In other exemplary embodiments, humanized anti-LIV1 IgG1 antibody-drug conjugates are provided.

In some embodiments, the invention provides an antibody-drug conjugate for the treatment of cancer. In some embodiments, the antibody-drug conjugate comprises an antibody conjugated to an auristatin. In some embodiments, the auristatin is a monomethyl auristatin. In some embodiments, the monomethyl auristatin is monomethyl auristatin E.

Unless otherwise indicated, an anti-LIV1-antibody drug conjugate (i.e., a LIV1-ADC) includes an antibody specific for the human LIV1 protein conjugated to a cytotoxic agent.

SGN-LIV1A is an anti-LIV1 humanized antibody (also referred to as hLIV22) which is conjugated to monomethyl auristatin E (MMAE) via a protease-cleavable linker (i.e., a valine-citrulline linker). Upon binding to a LIV1 expressing cell, SGN-LIV1A is internalized and releases MMAE, which disrupts microtubulin and induces apoptosis. SGN-LIV1A is also known as ladiratuzumab vedotin.

SGN-LIV1A comprises a humanized form of the mouse BR2-22a antibody, described in U.S. Pat. No. 9,228,026. Methods of making the SGN-LIV1A antibody are also disclosed in U.S. Pat. No. 9,228,026, which is incorporated herein by reference in its entirety for all purposes.

The amino acid sequence of the heavy chain variable region of SGN-LIV1A is provided herein as SEQ ID NO: 1. The amino acid sequence of the light chain variable region of SGN-LIV1A is provided herein as SEQ ID NO: 2. Synthesis and conjugation of the drug linker vcMMAE (shown below; also referred to as 1006) are further described in U.S. Pat. No. 9,228,026 and US Patent Pub. No. 2005/0238649, which are incorporated herein by reference in their entireties for all purposes.

TABLE 1
HCVR of SGN-LIV1A (SEQ ID NO: 1).

Gln Val Gln Leu Val Gln Ser Gly Ala Glu Val Lys
Lys Pro Gly Ala Ser Val Lys Val Ser Cys Lys Ala
Ser Gly Leu Thr Ile Glu Asp Tyr Tyr Met His Trp
Val Arg Gln Ala Pro Gly Gln Gly Leu Glu Trp Met
Gly Trp Ile Asp Pro Glu Asn Gly Asp Thr Glu Tyr
Gly Pro Lys Phe Gln Gly Arg Val Thr Met Thr Arg
Asp Thr Ser Ile Asn Thr Ala Tyr Met Glu Leu Ser
Arg Leu Arg Ser Asp Asp Thr Ala Val Tyr Tyr Cys
Ala Val His Asn Ala His Tyr Gly Thr Trp Phe Ala
Tyr Trp Gly Gln Gly Thr Leu Val Thr Val Ser Ser

TABLE 2
LCVR of SGN-LIV1A (SEQ ID NO: 2).

Asp Val Val Met Thr Gln Ser Pro Leu Ser Leu Pro
Val Thr Leu Gly Gln Pro Ala Ser Ile Ser Cys Arg
Ser Ser Gln Ser Leu Leu His Ser Ser Gly Asn Thr
Tyr Leu Glu Trp Tyr Gln Gln Arg Pro Gly Gln Ser
Pro Arg Pro Leu Ile Tyr Lys Ile Ser Thr Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Gly
Ser His Val Pro Tyr Thr Phe Gly Gly Gly Thr Lys
Val Glu Ile Lys Arg

According to certain exemplary embodiments, a LIV1-ADC comprises monomethyl auristatin E (MMAE) (PubChem CID: 53297465):

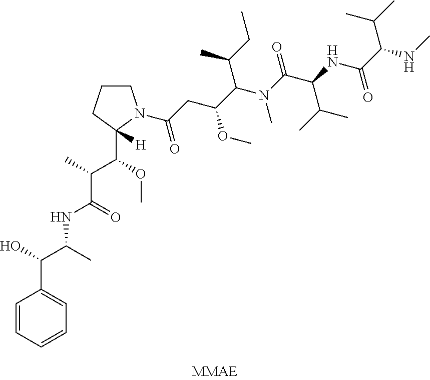 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

According to certain exemplary embodiments, a LIV1-ADC comprises vcMMAE conjugated thereto. vcMMAE is a drug-linker conjugate for ADC with potent anti-tumor activity comprising the anti-mitotic agent, MMAE, linked via the lysosomally cleavable dipeptide valine-citrulline (vc):

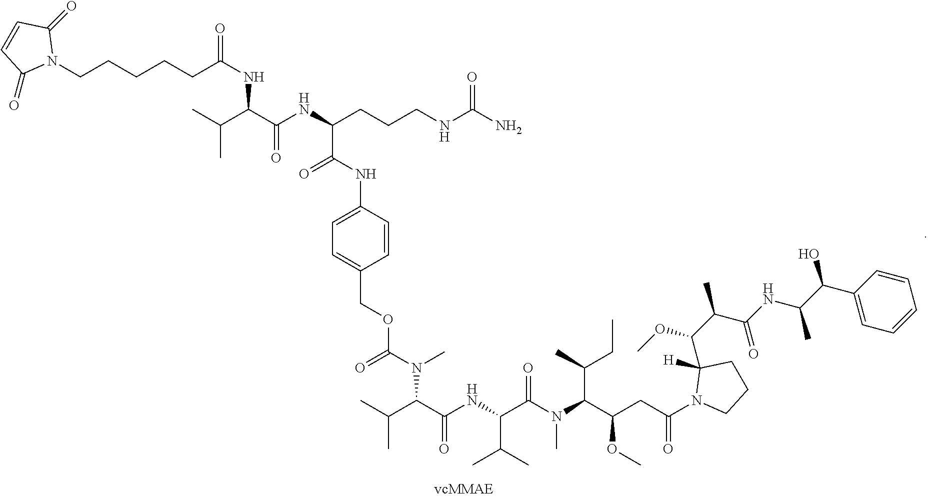 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

U.S. Pat. No. 9,228,026 discloses methods for conjugating vcMMAE to hLIV22.

A vcMMAE-antibody conjugate (e.g., a LIV1-ADC) according to certain exemplary embodiments is set forth below.

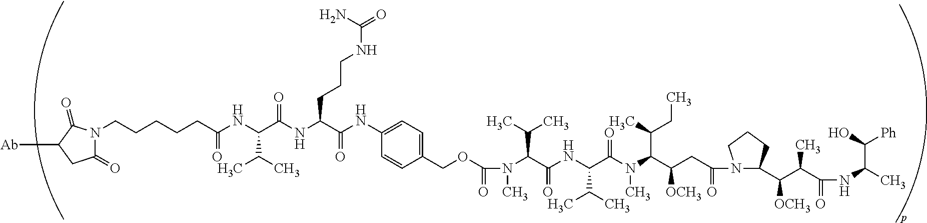

According to certain exemplary embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p may be any integer from about 1 to about 8. In some embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 1, representing a vcMMAE to antibody or antigen-binding fragment thereof ratio of 1. In some embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 2, 3, 4, 5, 6, 7, 8, 9, or 10, representing a vcMMAE to antibody or antigen-binding fragment thereof ratio (also known as a “Drug-to-Antibody Ratio” or “DAR”) of 2, 3, 4, 5, 6, 7, 8, 9, or 10, respectively. Accordingly, in some embodiments, a vcMMAE-antibody conjugate (e.g., a LIV1-ADC) is provided as set forth above, wherein a vcMMAE to antibody or antigen-binding fragment thereof ratio is 1, 2, 3, 4, 5, 6, 7, 8, 9, or 10. In certain exemplary embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 4, representing a vcMMAE to antibody or antigen-binding fragment thereof ratio of 4. Accordingly, in certain exemplary embodiments, a vcMMAE-antibody conjugate (e.g., a LIV1-ADC) is provided as set forth above, wherein a vcMMAE to antibody or antigen-binding fragment thereof ratio is 4.

SGN-LIV1A can be administered to subjects at a level that inhibits cancer cell growth, while at the same time is tolerated by the subject.

In certain exemplary embodiments, an anti-LIV1 antibody or antigen-binding fragment thereof comprises CDRs from an HCVR set forth as SEQ ID NO: 1 and/or CDRs from an LCVR set forth as SEQ ID NO: 2. In certain exemplary embodiments, an anti-LIV1 antibody or antigen-binding fragment thereof comprises an HCVR set forth as SEQ ID NO: 1 and/or an LCVR set forth as SEQ ID NO: 2. In other embodiments, an anti-LIV1 antibody or antigen-binding fragment thereof comprises an HCVR/LCVR pair SEQ ID NO: 1/SEQ ID NO: 2. In other embodiments, an anti-LIV1 antibody or antigen-binding fragment thereof comprises an HCVR that has at least about 80% homology or identity (e.g., 80%, 85%, 90%, 91%, 92%, 93%, 94%, 95%, 96%, 97%, 98% or 99%) to SEQ ID NO: 1 and/or comprises an LCVR that has at least about 80% homology or identity (e.g., 80%, 85%, 90%, 91%, 92%, 93%, 94%, 95%, 96%, 97%, 98% or 99%) to SEQ ID NO: 2.

Antibodies and antigen-binding fragments thereof and antibody-drug conjugates described herein (e.g., anti-LIV1 antibodies or LIV1-ADCs) can be expressed in a modified form. For instance, a region of additional amino acids, particularly charged amino acids, can be added to the N-terminus of an antibody or an antigen-binding fragment thereof or antibody-drug conjugates (e.g., a LIV1-ADC) to improve stability and persistence in the host cell, during purification, or during subsequent handling and storage. Also, peptide moieties can be added to an antibody or an antigen-binding fragment thereof or antibody-drug conjugates (e.g., a LIV1-ADC) of the present invention to facilitate purification. Such regions can be removed prior to final preparation of an antibody molecule or at least one fragment thereof. Such methods are described in many standard laboratory manuals, such as Sambrook, supra; Ausubel, et al., ed., Current Protocols In Molecular Biology, John Wiley & Sons, Inc., NY, N.Y. (1987-2001).

The antibodies or antigen-binding fragments thereof or antibody-drug conjugates (e.g., anti-LIV1 antibodies or LIV1-ADCs) described herein typically bind the target antigen (e.g., LIV1) with an equilibrium binding constant of about ≤1 μM, e.g., about ≤100 nM, about 10 nM, or about 1 nM, as measured using standard binding assays, for example, a Biacore-based binding assay.

Antibody molecules of the present invention may be characterized relative to a reference anti-LIV1 antibody, for example, BR2-22a. Antibody BR2-22a is described in U.S. Pat. No. 8,591,863 and is commercially available from American Type Culture Collection.

### Antibody-Drug Conjugates

In certain embodiments, the antibodies of the invention (e.g., anti-LIV1 antibodies) can be conjugated to a drug to form antibody-drug conjugates (ADCs). An exemplary anti-LIV1-ADC antibody is SGN-LIV1A. Particular ADCs may comprise cytotoxic agents (e.g., chemotherapeutic agents), prodrug converting enzymes, radioactive isotopes or compounds, or toxins (these moieties being collectively referred to as a therapeutic agent). For example, an ADC can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA replication inhibitors, chemotherapy sensitizers, DNA alkylating agents, and tubulin inhibitors. Other exemplary classes of cytotoxic agents include anthracyclines, auristatins, camptothecins, duocarmycins, etoposides, maytansinoids and vinca alkaloids. Some exemplary cytotoxic agents include auristatins (e.g., auristatin T, auristatin E, AFP, monomethyl auristatin F (MMAF), lipophilic monomethyl aurstatin F, monomethyl auristatin E (MMAE)), DNA minor groove binders (e.g., enediynes and lexitropsins), duocarmycins, taxanes (e.g., paclitaxel and docetaxel), vinca alkaloids, nicotinamide phosphoribosyltranferase inhibitor (NAMPTi), tubulysin M, doxorubicin, morpholino-doxorubicin, and cyanomorpholino-doxorubicin.

The cytotoxic agent can be a chemotherapeutic such as, for example, doxorubicin, paclitaxel, melphalan, vinca alkaloids, methotrexate, mitomycin C or etoposide. The agent can also be a CC-1065 analogue, calicheamicin, maytansine, an analog of dolastatin 10, rhizoxin, or palytoxin.

The cytotoxic agent can also be an auristatin. The auristatin can be an auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatins include auristatin T, AFP, MMAF, and MMAE. The synthesis and structure of various auristatins are described in, for example, US 2005-0238649 and US2006-0074008.

The cytotoxic agent can be a DNA minor groove binding agent. (See, e.g., U.S. Pat. No. 6,130,237.) For example, the minor groove binding agent can be a CBI compound or an enediyne (e.g., calicheamicin).

The cytotoxic or cytostatic agent can be an anti-tubulin agent. Examples of anti-tubulin agents include taxanes (e.g., Taxol® (paclitaxel), Taxotere® (docetaxel)), T67 (Tularik), vinca alkyloids (e.g., vincristine, vinblastine, vindesine, and vinorelbine), and auristatins (e.g., auristatin E, AFP, MMAF, MMAE, AEB, AEVB). Other suitable antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermoide and eleuthrobin.

The cytotoxic agent can be a maytansinoid, another group of anti-tubulin agents (e.g., DM1, DM2, DM3, DM4). For example, the maytansinoid can be maytansine or a maytansine containing drug linker such as DM-1 or DM-4 (ImmunoGen, Inc.; see also Chari et al., 1992, Cancer Res.).

An ADC can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14: 1-9; Senter, Cancer J., 2008, 14(3): 154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the LIV1-expressing cancer cell but is not substantially sensitive to the extracellular environment, such that the conjugate is cleaved from the antibody when it is internalized by the LIV1-expressing cancer cell (e.g., in the endosomal or, for example by virtue of pH sensitivity or protease sensitivity, in the lysosomal environment or in the caveolear environment). In some embodiments, the therapeutic agent can also be attached to the antibody with a non-cleavable linker.

In certain exemplary embodiments, an ADC can include a linker region between a cytotoxic or cytostatic agent and the antibody. As noted supra, typically, the linker can be cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in LIV1-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

A cleavable linker can be pH-sensitive, i.e., sensitive to hydrolysis, at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al, Biol. Chem. 264: 14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

A linker can also be a malonate linker (Johnson et al., Anticancer Res. 15: 1387-93, 1995), a maleimidobenzoyl linker (Lau et al., Bioorg-Med-Chem. 3: 1299-1304, 1995), or a 3′-N-amide analog (Lau et al., Bioorg-Med-Chem. 3: 1305-12, 1995).

A linker also can be a non-cleavable linker, such as a maleimido-alkylene or maleimide-aryl linker that is directly attached to the therapeutic agent and released by proteolytic degradation of the antibody.

Typically, a linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the ADC are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

A linker can also promote cellular internalization, e.g., when conjugated to the therapeutic agent (i.e., in the milieu of the linker-therapeutic agent moiety of the ADC or ADC derivate as described herein). Alternatively, the linker can promote cellular internalization when conjugated to both the therapeutic agent and the antibody (i.e., in the milieu of the ADC as described herein).

An antibody (e.g., anti-LIV1 antibody) can be conjugated to a linker via a heteroatom of the antibody. These heteroatoms can be present on the antibody in its natural state or can be introduced into the antibody (e.g., anti-LIV1 antibody). In some aspects, the antibody (e.g., anti-LIV1 antibody) will be conjugated to the linker via a sulfur atom of a cysteine residue. Methods of conjugating linker and drug-linkers to antibodies are known in the art.

Exemplary antibody-drug conjugates include auristatin based antibody-drug conjugates meaning that the drug component is an auristatin drug. Auristatins bind tubulin, have been shown to interfere with microtubule dynamics and nuclear and cellular division, and have anticancer activity. Typically the auristatin based antibody-drug conjugate comprises a linker between the auristatin drug and the antibody (e.g., anti-LIV1 antibody). The linker can be, for example, a cleavable linker (e.g., a peptidyl linker) or a non-cleavable linker (e.g., linker released by degradation of the antibody). Auristatins include MMAF and MMAE. The synthesis and structure of exemplary auristatins are described in U.S. Pat. Nos. 7,659,241, 7,498,298, 7,968,687, and U.S. Pub. Nos. 2009/0111756 and 2009/0018086, each of which is incorporated herein by reference in its entirety and for all purposes.

In certain embodiments, an antibody or antigen-binding fragment thereof can be conjugated to a drug to form an antibody-drug conjugate (ADC) and may have a ratio of drug moieties per antibody of about 1 to about 8. In certain embodiments, an antibody or antigen-binding fragment thereof (e.g., anti-LIV1 antibody) can be conjugated to a drug to form an ADC and may have a ratio of drug moieties per antibody of about 2 to about 5. In some embodiments, the ratio of drug moieties per antibody is 1, 2, 3, 4, 5, 6, 7, 8, 9, or 10. In certain exemplary embodiments, an anti-LIV1 antibody or antigen-binding fragment thereof can be conjugated to a drug to form an ADC and have a ratio of drug moieties per antibody of about 4. In some embodiments, the average number of drug moieties per antibody in a population of antibody-drug conjugates is about 1 to about 8. In some embodiments, the average number of drug moieties per antibody in a population of antibody-drug conjugates is about 4. Methods of determining the ratio of drug moieties per antibody or antigen-binding fragment thereof of an ADC are readily known to those skilled in the art.

### III. Therapeutic Applications

The invention provides methods of treating disorders associated with cells that express LIV1, e.g., cancers. In one aspect, the invention provides the use of humanized anti-LIV1 antibodies and antigen-binding fragments or conjugates thereof (e.g., anti-LIV1-antibody-drug conjugates (anti-LIV1-ADCs) for the treatment of cancers, such as solid tumors, such as, e.g., locally advanced or metastatic solid tumors (e.g., small cell lung cancer, non-small cell lung cancer, head and neck squamous cell carcinoma, esophageal squamous cell carcinoma, and gastric and gastroesophageal junction adenocarcinoma). In one aspect, the invention provides the use of humanized anti-LIV1 antibodies and antigen-binding fragments or conjugates thereof (e.g., anti-LIV1-antibody-drug conjugates (anti-LIV1-ADCs) for the treatment of cancers, such as breast cancer.

As used herein, the terms “subject” and “patient” refer to organisms to be treated by the methods of the present invention. Such organisms preferably include, but are not limited to, mammals (e.g., murines, simians, equines, bovines, porcines, canines, felines, and the like), and more preferably includes humans. As used herein, the terms “treat,” “treatment” and “treating” include any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

Positive therapeutic effects in cancer can be measured in a number of ways (See, W. A. Weber, J. Null. Med. 50:1S-10S (2009); Eisenhauer et al., supra). In certain exemplary embodiments, response to an anti-LIV1 antibody or an antigen-binding fragment thereof (e.g., a LIV1-ADC) is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat a cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Tumor” as it applies to a subject diagnosed with, or suspected of having, cancer (e.g., solid cancer or breast cancer), refers to a malignant or potentially malignant neoplasm or tissue mass of any size.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

As used herein, the term “effective amount” refers to the amount of a compound (e.g., an anti-LIV1 antibody or antigen-binding fragment thereof or antibody-drug conjugate) sufficient to effect beneficial or desired results. An effective amount of an antibody or antigen-binding fragment thereof or antibody-drug conjugate (e.g., a LIV1-ADC) can be administered in one or more administrations, applications or dosages and is not intended to be limited to a particular formulation or administration route. Generally, a therapeutically effective amount of an antibody or antigen-binding fragment thereof or antibody-drug conjugate (e.g., a LIV1-ADC) is in the range of 0.2 mg/kg to 3.5 mg/kg of body weight of the subject. In some embodiments, the maximum dose is about 100 mg, about 125 mg, about 200 mg or about 250 mg. In some embodiments, a therapeutically effective amount of an antibody or antigen-binding fragment thereof or antibody-drug conjugate (e.g., a LIV1-ADC) is in the range of 0.5 mg/kg to 2.8 mg/kg at a maximum dose of about 100 mg, about 125 mg, about 200 mg, or about 250 mg. In some embodiments, a therapeutically effective amount of an antibody or antigen-binding fragment thereof or antibody-drug conjugate (e.g., a LIV1-ADC) is in the range of 0.5 mg/kg to 3.0 mg/kg of body weight of the subject at a maximum dose of about 200 mg. In some embodiments, a therapeutically effective amount of an antibody or antigen-binding fragment thereof or antibody-drug conjugate (e.g., a LIV1-ADC) is in the range of 0.5 mg/kg to 2.0 mg/kg of body weight of the subject. In some embodiments, a therapeutically effective amount of an antibody or antigen-binding fragment thereof or antibody-drug conjugate (e.g., a LIV1-ADC) is in the range of 0.75 mg/kg to 1.67 mg/kg of body weight of the subject. In some embodiments, a therapeutically effective amount of an antibody or antigen-binding fragment thereof or antibody-drug conjugate (e.g., a LIV1-ADC) is in the range of 0.5 mg/kg to 3.0 mg/kg of body weight of the subject. In some embodiments, a therapeutically effective amount of an antibody or antigen-binding fragment thereof or antibody-drug conjugate (e.g., a LIV1-ADC) is in the range of 1.0 mg/kg to 2.5 mg/kg of body weight of the subject. In some embodiments, a therapeutically effective amount of an antibody or antigen-binding fragment thereof or antibody-drug conjugate (e.g., a LIV1-ADC) is about 0.75 mg/kg of body weight of the subject. In some embodiments, a therapeutically effective amount of an antibody or antigen-binding fragment thereof or antibody-drug conjugate (e.g., a LIV1-ADC) is 0.75 mg/kg of body weight of the subject. In some embodiments, a therapeutically effective amount of an antibody or antigen-binding fragment thereof or antibody-drug conjugate (e.g., a LIV1-ADC) is about 1.0 mg/kg of body weight of the subject. In some embodiments, a therapeutically effective amount of an antibody or antigen-binding fragment thereof or antibody-drug conjugate (e.g., a LIV1-ADC) is 1.0 mg/kg of body weight of the subject. In some embodiments, a therapeutically effective amount of an antibody or antigen-binding fragment thereof or antibody-drug conjugate (e.g., a LIV1-ADC) is about 1.25 mg/kg of body weight of the subject. In some embodiments, a therapeutically effective amount of an antibody or antigen-binding fragment thereof or antibody-drug conjugate (e.g., a LIV1-ADC) is 1.25 mg/kg of body weight of the subject. In some embodiments, a therapeutically effective amount of an antibody or antigen-binding fragment thereof or antibody-drug conjugate (e.g., a LIV1-ADC) is about 1.5 mg/kg of body weight of the subject. In some embodiments, a therapeutically effective amount of an antibody or antigen-binding fragment thereof or antibody-drug conjugate (e.g., a LIV1-ADC) is 1.5 mg/kg of body weight of the subject. In some embodiments, a therapeutically effective amount of an antibody or antigen-binding fragment thereof or antibody-drug conjugate (e.g., a LIV1-ADC) is about 1.67 mg/kg of body weight of the subject. In some embodiments, a therapeutically effective amount of an antibody or antigen-binding fragment thereof or antibody-drug conjugate (e.g., a LIV1-ADC) is 1.67 mg/kg of body weight of the subject. In some embodiments, a therapeutically effective amount of an antibody or antigen-binding fragment thereof or antibody-drug conjugate (e.g., a LIV1-ADC) is about 1.75 mg/kg of body weight of the subject. In some embodiments, a therapeutically effective amount of an antibody or antigen-binding fragment thereof or antibody-drug conjugate (e.g., a LIV1-ADC) is 1.75 mg/kg of body weight of the subject. In some embodiments, a therapeutically effective amount of an antibody or antigen-binding fragment thereof or antibody-drug conjugate (e.g., a LIV1-ADC) is about 2.0 mg/kg of body weight of the subject. In some embodiments, a therapeutically effective amount of an antibody or antigen-binding fragment thereof or antibody-drug conjugate (e.g., a LIV1-ADC) is 2.0 mg/kg of body weight of the subject. In some embodiments, a therapeutically effective amount of an antibody or antigen-binding fragment thereof or antibody-drug conjugate (e.g., a LIV1-ADC) is about 2.25 mg/kg of body weight of the subject. In some embodiments, a therapeutically effective amount of an antibody or antigen-binding fragment thereof or antibody-drug conjugate (e.g., a LIV1-ADC) is 2.5 mg/kg of body weight of the subject. The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment. Dosing frequency can vary, depending on factors such as route of administration, dosage amount, serum half-life of the antibody, and the disease being treated. Exemplary dosing frequencies are once per day, once per week, once every two weeks and once every three weeks. Formulation of monoclonal antibody-based drugs is within ordinary skill in the art. In some embodiments, a monoclonal antibody is lyophilized, and then reconstituted in buffered saline, at the time of administration.

In some embodiments, an antibody or antigen-binding fragment thereof or antibody-drug conjugate (e.g., a LIV1-ADC) is administered to a patient who failed to achieve a sustained response after prior therapy (e.g., after failed or ineffective therapy with a systemic anti-cancer therapy that is not the antibody or antigen-binding fragment thereof or antibody-drug conjugate (e.g., a LIV1-ADC)), i.e., is cancer treatment-experienced.

In some embodiments, a medicament comprising an antibody or antigen-binding fragment thereof or antibody-drug conjugate (e.g., a LIV1-ADC), as described above, may be provided as a liquid formulation or prepared by reconstituting a lyophilized powder with sterile water for injection prior to use.

In certain embodiments, the dosing regimen will comprise administering an anti-LIV1 antibody or antigen-binding fragment thereof or antibody-drug conjugate (e.g., a LIV1-ADC) once about every week, once about every two weeks, once about every three weeks or once about every month.

In certain embodiments, the dosing regimen will comprise administering an anti-LIV1 antibody or antigen-binding fragment thereof or antibody-drug conjugate (e.g., a LIV1-ADC) at a dose of about 2.5 mg/kg of a subject's body weight at intervals of about 21 days (±2 days) throughout the course of treatment. In certain embodiments, an anti-LIV1 antibody or antigen-binding fragment thereof (e.g., a LIV1-ADC) is used at a dose of less than about 200 mg every 3 weeks.

In certain embodiments, the dosing regimen will comprise administering an anti-LIV1 antibody or antigen-binding fragment thereof or antibody-drug conjugate (e.g., a LIV1-ADC) at a dose of about 1.0 mg/kg of a subject's body weight at intervals of about 7 days (±1 day) throughout the course of treatment. In certain embodiments, an anti-LIV1 antibody or antigen-binding fragment thereof (e.g., a LIV1-ADC) is used at a dose of less than about 100 mg every 1 week.

In certain embodiments, the dosing regimen will comprise administering an anti-LIV1 antibody or antigen-binding fragment thereof or antibody-drug conjugate (e.g., a LIV1-ADC) at a dose of about 1.25 mg/kg of a subject's body weight at intervals of about 7 days (±1 day) throughout the course of treatment. In certain embodiments, an anti-LIV1 antibody or antigen-binding fragment thereof (e.g., a LIV1-ADC) is used at a dose of less than about 125 mg every 1 week.

In certain embodiments, the dosing regimen will comprise administering an anti-LIV1 antibody or antigen-binding fragment thereof or antibody-drug conjugate (e.g., a LIV1-ADC) at a dose of about 2.5 mg/kg of a subject's body weight at intervals of about 21 days (±2 days) throughout the course of treatment. In certain embodiments, an anti-LIV1 antibody or antigen-binding fragment thereof or antibody-drug conjugate (e.g., a LIV1-ADC) is used at a dose of less than or equal to about 250 mg every 3 weeks. In certain embodiments, an anti-LIV1 antibody or antigen-binding fragment thereof or antibody-drug conjugate (e.g., a LIV1-ADC) is used at a dose of less than or equal to 250 mg every 3 weeks. In certain embodiments, the subject is further administered granulocyte colony stimulating factor (GCSF). In certain embodiments, if the anti-LIV1 antibody or antigen-binding fragment thereof or antibody-drug conjugate (e.g., a LIV1-ADC) is used at a dose of greater than or equal to about 200 mg and less than or equal to about 250 mg every 3 weeks, the subject is further administered GCSF. In certain embodiments, if the anti-LIV1 antibody or antigen-binding fragment thereof or antibody-drug conjugate (e.g., a LIV1-ADC) is used at a dose of greater than or equal to 200 mg and less than or equal to 250 mg every 3 weeks, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In certain embodiments, the dosing regimen will comprise administering an anti-LIV1 antibody or antigen-binding fragment thereof or antibody-drug conjugate (e.g., a LIV1-ADC) at a dose of about 1.0 mg/kg of a subject's body weight at intervals of about 7 days (±1 day) throughout the course of treatment. In certain embodiments, an anti-LIV1 antibody or antigen-binding fragment thereof or antibody-drug conjugate (e.g., a LIV1-ADC) is used at a dose of less than or equal to about 100 mg every 1 week. In certain embodiments, an anti-LIV1 antibody or antigen-binding fragment thereof or antibody-drug conjugate (e.g., a LIV1-ADC) is used at a dose of less than or equal to 100 mg every 1 week. In certain embodiments, the subject is further administered granulocyte colony stimulating factor (GCSF). In certain embodiments, if the anti-LIV1 antibody or antigen-binding fragment thereof or antibody-drug conjugate (e.g., a LIV1-ADC) is used at a dose of greater than or equal to about 80 mg and less than or equal to about 100 mg every 1 week, the subject is further administered GCSF. In certain embodiments, if the anti-LIV1 antibody or antigen-binding fragment thereof or antibody-drug conjugate (e.g., a LIV1-ADC) is used at a dose of greater than or equal to 80 mg and less than or equal to 100 mg every 1 week, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In certain embodiments, the dosing regimen will comprise administering an anti-LIV1 antibody or antigen-binding fragment thereof or antibody-drug conjugate (e.g., a LIV1-ADC) at a dose of about 1.25 mg/kg of a subject's body weight at intervals of about 7 days (±1 day) throughout the course of treatment. In certain embodiments, an anti-LIV1 antibody or antigen-binding fragment thereof or antibody-drug conjugate (e.g., a LIV1-ADC) is used at a dose of less than or equal to about 125 mg every 1 week. In certain embodiments, an anti-LIV1 antibody or antigen-binding fragment thereof or antibody-drug conjugate (e.g., a LIV1-ADC) is used at a dose of less than or equal to 125 mg every 1 week. In certain embodiments, the subject is further administered granulocyte colony stimulating factor (GCSF). In certain embodiments, if the anti-LIV1 antibody or antigen-binding fragment thereof or antibody-drug conjugate (e.g., a LIV1-ADC) is used at a dose of greater than or equal to about 100 mg and less than or equal to about 125 mg every 1 week, the subject is further administered GCSF. In certain embodiments, if the anti-LIV1 antibody or antigen-binding fragment thereof or antibody-drug conjugate (e.g., a LIV1-ADC) is used at a dose of greater than or equal to 100 mg and less than or equal to 125 mg every 1 week, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In certain embodiments, the dosing regimen will comprise administering an antibody or antigen-binding fragment thereof or antibody-drug conjugate (e.g., a LIV1-ADC) once about every week, once about every two weeks, once about every three weeks or once about every month. In certain embodiments, the subject is further administered granulocyte colony-stimulating factor (GCSF). In certain embodiments, if the dose is greater than or equal to about 200 mg and less than or equal to about 250 mg Q3W or Q21D, the subject is further administered GCSF. In certain embodiments, the subject is further administered GCSF. In certain embodiments, if the dose is greater than or equal to 200 mg and less than or equal to 250 mg, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In certain embodiments, the dosing regimen will comprise administering an antibody or antigen-binding fragment thereof or antibody-drug conjugate (e.g., a LIV1-ADC) at a dose of about 0.5 mg/kg to about 2.0 mg/kg of body weight of the subject at intervals of about 7 days (±1 day) throughout the course of treatment. In certain embodiments, the dosing regimen will comprise administering an antibody or antigen-binding fragment thereof or antibody-drug conjugate (e.g., a LIV1-ADC) at a dose of about 0.5 mg/kg to about 3.0 mg/kg of body weight of the subject twice in a three week treatment cycle throughout the course of treatment. In some embodiments, the antibody or antigen-binding fragment thereof or antibody-drug conjugate (e.g., a LIV1-ADC) is administered on day 1 and day 8 of the three week treatment cycle (D1 & D8 Q3W). Increasing the frequency of antibody-drug conjugate dosing, such as by the use of weekly dosing or administration on day 1 and day 8 of a three week treatment cycle (D1 & D8 Q3W), may result in the ability to administer higher total doses and improved efficacy while potentially incurring only minimal worsening of certain adverse events. As shown in the examples, when antibody-drug conjugates are administered less frequently, such as once every three weeks, the concentration of antibody-drug conjugates with a high drug to antibody ratio (DAR) drops to zero between doses due to fast clearance. More frequent administration, such as administration every week or on day 1 and day 8 of a three week treatment cycle (D1 & D8 Q3W) may result in smaller peak to trough fluctuations and maintain higher C_{trough} values throughout the course of treatment, which could improve treatment efficacy. Furthermore, the elimination of antibody-drug conjugate administration on D15 of a D1 & D8 Q3W treatment cycle enables hematologic recovery and may prevent dose delays and dose elimination.

In a particular embodiment of the invention, the antibody or antigen-binding fragment thereof or antibody-drug conjugate (e.g., a LIV1-ADC) is administered to the subject a dose selected from the group consisting of about 0.5 mg/kg of body weight every week or every 7 days (±1 day), about 0.6 mg/kg of body weight every week or every 7 days (±1 day), about 0.7 mg/kg of body weight every week or every 7 days (±1 day), about 0.75 mg/kg of body weight every week or every 7 days (±1 day), about 0.8 mg/kg of body weight every week or every 7 days (±1 day), about 0.9 mg/kg of body weight every week or every 7 days (±1 day), about 1.0 mg/kg of body weight every week or every 7 days (±1 day), about 1.1 mg/kg of body weight every week or every 7 days (±1 day), about 1.2 mg/kg of body weight every week or every 7 days (±1 day), about 1.25 mg/kg of body weight every week or every 7 days (±1 day), about 1.3 mg/kg of body weight every week or every 7 days (±1 day), about 1.4 mg/kg of body weight every week or every 7 days (±1 day), about 1.5 mg/kg of body weight every week or every 7 days (±1 day), about 1.6 mg/kg of body weight every week or every 7 days (±1 day), about 1.67 mg/kg of body weight every week or every 7 days (±1 day), about 1.7 mg/kg of body weight every week or every 7 days (±1 day), about 1.75 mg/kg of body weight every week or every 7 days (±1 day), about 1.8 mg/kg of body weight every week or every 7 days (±1 day), about 1.9 mg/kg of body weight every week or every 7 days (±1 day), and about 2.0 mg/kg of body weight every week or every 7 days (±1 day), and maximum equivalents of any of these doses, such as, e.g., less than about 200 mg every week or every 7 days (±1 day).

In a particular embodiment of the invention, the antibody or antigen-binding fragment thereof or antibody-drug conjugate (e.g., a LIV1-ADC) is administered to the subject a dose selected from the group consisting of 0.5 mg/kg of body weight every week or every 7 days (±1 day), 0.6 mg/kg of body weight every week or every 7 days (±1 day), 0.7 mg/kg of body weight every week or every 7 days (±1 day), 0.75 mg/kg of body weight every week or every 7 days (±1 day), 0.8 mg/kg of body weight every week or every 7 days (±1 day), 0.9 mg/kg of body weight every week or every 7 days (±1 day), 1.0 mg/kg of body weight every week or every 7 days (±1 day), 1.1 mg/kg of body weight every week or every 7 days (±1 day), 1.2 mg/kg of body weight every week or every 7 days (±1 day), 1.25 mg/kg of body weight every week or every 7 days (±1 day), 1.3 mg/kg of body weight every week or every 7 days (±1 day), 1.4 mg/kg of body weight every week or every 7 days (±1 day), 1.5 mg/kg of body weight every week or every 7 days (±1 day), 1.6 mg/kg of body weight every week or every 7 days (±1 day), 1.67 mg/kg of body weight every week or every 7 days (±1 day), 1.7 mg/kg of body weight every week or every 7 days (±1 day), 1.75 mg/kg of body weight every week or every 7 days (±1 day), 1.8 mg/kg of body weight every week or every 7 days (±1 day), 1.9 mg/kg of body weight every week or every 7 days (±1 day), and 2.0 mg/kg of body weight every week or every 7 days (±1 day), and maximum equivalents of any of these doses, such as, e.g., less than about 200 mg every week or every 7 days (±1 day).

In a particular embodiment of the invention, the antibody or antigen-binding fragment thereof or antibody-drug conjugate (e.g., a LIV1-ADC) is administered to the subject a dose selected from the group consisting of about 0.5 mg/kg of body weight twice every three weeks (±3 days), about 0.75 mg/kg of body weight twice every three weeks (±3 days), about 1.0 mg/kg of body weight twice every three weeks (±3 days), about 1.25 mg/kg of body weight twice every three weeks (±3 days), about 1.5 mg/kg of body weight twice every three weeks (±3 days), about 1.75 mg/kg of body weight twice every three weeks (±3 days), about 2.0 mg/kg of body weight twice every three weeks (±3 days), about 2.25 mg/kg of body weight twice every three weeks (±3 days), about 2.5 mg/kg of body weight twice every three weeks (±3 days), about 2.75 mg/kg of body weight twice every three weeks (±3 days), and about 3.0 mg/kg of body weight twice every three weeks (±3 days), and maximum equivalents of any of these doses, such as, e.g., less than about 200 mg every 3 weeks (±3 days). In some embodiments, the antibody or antigen-binding fragment thereof or antibody-drug conjugate (e.g., a LIV1-ADC) is administered on day 1 and day 8 of the three week treatment cycle (D1 & D8 Q3W).

In a particular embodiment of the invention, the antibody or antigen-binding fragment thereof or antibody-drug conjugate (e.g., a LIV1-ADC) is administered to the subject a dose selected from the group consisting of 0.5 mg/kg of body weight twice every three weeks (±3 days), 0.75 mg/kg of body weight twice every three weeks (±3 days), 1.0 mg/kg of body weight twice every three weeks (±3 days), 1.25 mg/kg of body weight twice every three weeks (±3 days), 1.5 mg/kg of body weight twice every three weeks (±3 days), 1.75 mg/kg of body weight twice every three weeks (±3 days), 2.0 mg/kg of body weight twice every three weeks (±3 days), 2.25 mg/kg of body weight twice every three weeks (±3 days), 2.5 mg/kg of body weight twice every three weeks (±3 days), 2.75 mg/kg of body weight twice every three weeks (±3 days), and 3.0 mg/kg of body weight twice every three weeks (±3 days), and maximum equivalents of any of these doses, such as, e.g., less than about 200 mg every 3 weeks (±3 days). In some embodiments, the antibody or antigen-binding fragment thereof or antibody-drug conjugate (e.g., a LIV1-ADC) is administered on day 1 and day 8 of the three week treatment cycle (D1 & D8 Q3W).

In certain exemplary embodiments, an antibody or antigen-binding fragment thereof or antibody-drug conjugate (e.g., a LIV1-ADC) is administered as a monotherapy. In certain exemplary embodiments, an antibody or antigen-binding fragment thereof or antibody-drug conjugate (e.g., a LIV1-ADC) is administered in combination with trastuzumab. In certain exemplary embodiments, an antibody or antigen-binding fragment thereof or antibody-drug conjugate (e.g., a LIV1-ADC) is administered in combination with a checkpoint inhibitor. In some embodiments, the checkpoint inhibitor is an anti-PD-1 antibody, an anti-PD-L1 antibody, an anti-CTLA4 antibody, B7-DC-Fc, LAG3, or TIM3. In some embodiments, the checkpoint inhibitor is selected from the group consisting of MEDI0680, AMP-224, nivolumab, pembrolizumab, pidilizumab, MEDI4736, MPDL3280A, ipilimumab and tremelimumab. In some embodiments, the checkpoint inhibitor is pembrolizumab.

In certain exemplary embodiments, the present invention provides a method for treating cancer in a cell, tissue, organ, animal or patient. In certain exemplary embodiments, the present invention provides a method for treating solid tumor in a human. In certain exemplary embodiments, the present invention provides a method for treating breast cancer in a human.

In certain embodiments, a subject will be administered a parenteral dosing, e.g., an intravenous (IV) infusion, of a medicament comprising an anti-LIV1 antibody or antigen-binding fragment thereof or antibody-drug conjugate (e.g., a LIV1-ADC).

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.5 mg/kg of body weight every three weeks (Q3W, Q3wk) or every 21 days (Q21D), about 1.0 mg/kg of body weight Q3W or Q21D, about 1.5 mg/kg of body weight Q3W or Q21D, about 2.0 mg/kg of body weight Q3W or Q21D, about 2.5 mg/kg of body weight Q3W or Q21D, about 2.8 mg/kg of body weight Q3W or Q21D, about 3.0 mg/kg of body weight Q3W or Q21D, about 3.2 mg/kg of body weight Q3W or Q21D, or about 3.5 mg/kg of body weight Q3W or Q21D, and maximum equivalents of any of these doses, such as, e.g., less than about 200 mg Q3W or Q21D.

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.5 mg/kg of body weight every three weeks (Q3W) or every 21 days (Q21D), about 1.0 mg/kg of body weight Q3W or Q21D, about 1.5 mg/kg of body weight Q3W or Q21D, about 2.0 mg/kg of body weight Q3W or Q21D, about 2.5 mg/kg of body weight Q3W or Q21D, about 2.8 mg/kg of body weight Q3W or Q21D, about 3.0 mg/kg of body weight Q3W or Q21D, about 3.2 mg/kg of body weight Q3W or Q21D, or about 3.5 mg/kg of body weight Q3W or Q21D, and maximum equivalents of any of these doses, such as, e.g., less than or equal to about 250 mg Q3W or Q21D. In certain embodiments, the subject is further administered GCSF. In certain embodiments, if the dose is greater than or equal to about 200 mg and less than or equal to about 250 mg Q3W or Q21D, the subject is further administered GCSF. In certain embodiments, if the dose is greater than or equal to 200 mg and less than or equal to 250 mg Q3W or Q21D, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.25 mg/kg of body weight every one week (Q1W, Q1wk) or every 7 days (Q7D), about 0.50 mg/kg of body weight Q1W or Q7D, about 0.75 mg/kg of body weight Q1W or Q7D, about 1.0 mg/kg of body weight Q1W or Q7D, about 1.25 mg/kg of body weight Q1W or Q7D, about 1.5 mg/kg of body weight Q1W or Q7D, or about 1.75 mg/kg of body weight Q1W or Q7D, and maximum equivalents of any of these doses, such as, e.g., less than about 100 mg Q1W or Q7D or less than about 125 mg Q1W or Q7D.

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.25 mg/kg of body weight every one week (Q1W) or every 7 days (Q7D), about 0.5 mg/kg of body weight Q1W or Q7D, about 0.75 mg/kg of body weight Q1W or Q7D, about 1.0 mg/kg of body weight Q1W or Q7D, about 1.25 mg/kg of body weight Q1W or Q7D, about 1.5 mg/kg of body weight Q1W or Q7D, or about 1.75 mg/kg of body weight Q1W or Q7D, and maximum equivalents of any of these doses, such as, e.g., less than or equal to about 100 mg Q1W or Q7D or less than about 125 mg Q1W or Q7D. In certain embodiments, the subject is further administered GCSF. In certain embodiments, if the dose is greater than or equal to about 80 mg and less than or equal to about 100 mg Q1W or Q7D, the subject is further administered GCSF. In certain embodiments, if the dose is greater than or equal to 80 mg and less than or equal to 100 mg Q1W or Q7D, the subject is further administered GCSF. In certain embodiments, if the dose is greater than or equal to about 100 mg and less than or equal to about 125 mg Q1W or Q7D, the subject is further administered GCSF. In certain embodiments, if the dose is greater than or equal to 100 mg and less than or equal to 125 mg Q1W or Q7D, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In some embodiments, an anti-LIV1 antibody or antigen-binding fragment thereof or antibody-drug conjugate (e.g., a LIV1-ADC) is provided in a dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 191 mg, about 192 mg, about 193 mg, about 194 mg, about 195 mg, about 196 mg, about 197 mg, about 198 mg, about 199 mg or about 200 mg. In certain exemplary embodiments, an anti-LIV1 antibody or antigen-binding fragment thereof or antibody-drug conjugate (e.g., a LIV1-ADC) is provided in a dosage of less than about 200 mg, e.g., at a dosage of about 200 mg, at a dosage of about 199 mg, about 198 mg, about 197 mg, about 196 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, about 100 mg, about 90 mg, about 80 mg, about 75 mg, about 60 mg, about 50 mg, about 40 mg, about 30 mg, about 25 mg, about 20 mg, or about 10 mg.

In some embodiments, an anti-LIV1 antibody or antigen-binding fragment thereof or antibody-drug conjugate (e.g., a LIV1-ADC) is provided in a dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 245 mg or about 250 mg. In certain exemplary embodiments, an anti-LIV1 antibody or antigen-binding fragment thereof or antibody-drug conjugate (e.g., a LIV1-ADC) is provided in a dosage of less than or equal to about 250 mg, e.g., at a dosage of about 250 mg, at a dosage of about 245 mg, about 240 mg, about 235 mg, about 230 mg, about 225 mg, about 220 mg, about 215 mg, about 210 mg, about 205 mg, about 200 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, about 100 mg, about 90 mg, about 80 mg, about 75 mg, about 60 mg, about 50 mg, about 40 mg, about 30 mg, about 25 mg, about 20 mg, or about 10 mg.

In certain exemplary embodiments, an anti-LIV1 antibody or antigen-binding fragment thereof (e.g., a LIV1-ADC) is administered as a monotherapy. In certain exemplary embodiments, an anti-LIV1 antibody or antigen-binding fragment thereof (e.g., a LIV1-ADC) is administered in combination with a checkpoint inhibitor. In some embodiments, the checkpoint inhibitor is an anti-PD-1 antibody, an anti-PD-L1 antibody, an anti-CTLA4 antibody, B7-DC-Fc, LAG3, or TIM3. In some embodiments, the checkpoint inhibitor is selected from the group consisting of MEDI0680, AMP-224, nivolumab, pembrolizumab, pidilizumab, MEDI4736, MPDL3280A, ipilimumab and tremelimumab. In some embodiments, the checkpoint inhibitor is pembrolizumab.

In certain exemplary embodiments, the present invention provides a method for treating cancer in a cell, tissue, organ, animal or patient. In certain exemplary embodiments, the present invention provides a method for treating solid tumors, such as, e.g., small cell lung cancer, non-small cell lung cancer, head and neck squamous cell carcinoma, esophageal squamous cell carcinoma, and gastric and gastroesophageal junction adenocarcinoma, or breast cancer in a human. In a particular exemplary embodiment, the small cell lung cancer, non-small cell lung cancer, head and neck squamous cell carcinoma, esophageal squamous cell carcinoma, gastric and gastroesophageal junction adenocarcinoma, or breast cancer is locally advance or metastatic.

In some embodiments, the subject has been previously treated for the small cell lung cancer, non-small cell lung cancer, head and neck squamous cell carcinoma, esophageal squamous cell carcinoma, gastric and gastroesophageal junction adenocarcinoma, or breast cancer. In some embodiments, the subject did not respond to the treatment (e.g., the subject experienced disease progression during treatment). In some embodiments, the subject relapsed after the treatment. In some embodiments, the subject experienced disease progression after the treatment. In some embodiments, the treatment previously administered to the subject was not an anti-LIV1 antibody or antigen-binding fragment thereof as described herein.

Certain small cell lung cancer, non-small cell lung cancer, head and neck squamous cell carcinoma, esophageal squamous cell carcinoma, gastric and gastroesophageal junction adenocarcinoma cancers or breast cancers show detectable levels of LIV1 measured at either the protein (e.g., by immunoassay using one of the exemplified antibodies) or the mRNA level. In certain embodiments, a small cell lung cancer, non-small cell lung cancer, head and neck squamous cell carcinoma, esophageal squamous cell carcinoma, gastric and gastroesophageal junction adenocarcinoma cancers or breast cancer shows elevated levels of LIV1 relative to non-cancerous tissue or cells of the same type, e.g., lung, squamous, esophageal, gastric and gastroesophageal, or other breast cells or tissues from the same patient. In other embodiments, a small cell lung cancer, non-small cell lung cancer, head and neck squamous cell carcinoma, esophageal squamous cell carcinoma, gastric and gastroesophageal junction adenocarcinoma cancers or breast cancer shows similar levels of LIV1 relative to non-cancerous lung, squamous, esophageal, gastric and gastroesophageal, or breast tissue or breast cells of the same type, e.g., from the same patient.

An exemplary level of LIV1 protein on small cell lung cancer, non-small cell lung cancer, head and neck squamous cell carcinoma, esophageal squamous cell carcinoma, gastric and gastroesophageal junction adenocarcinoma cancer or breast cancer cells amenable to treatment is 5,000-150,000 LIV1 proteins per cell, although breast cancers associated with higher or lower levels can be treated. Optionally, LIV1 levels (e.g., LIV1 protein levels) in a small cell lung cancer, non-small cell lung cancer, head and neck squamous cell carcinoma, esophageal squamous cell carcinoma, gastric and gastroesophageal junction adenocarcinoma cancer or breast cancer from a subject are measured before performing treatment. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express LIV1.

A. Lung Cancer

Lung cancer remains the leading cause of death from cancer in the United States, with over 155,000 deaths estimated in 2017. Treatments with curative intent for patients with early stage disease include surgery, chemotherapy, radiation therapy, or a combined modality approach. However, a majority of patients are diagnosed with advanced stage disease, which is usually incurable. Non-small cell lung cancer (NSCLC) represents up to 80% of all lung cancers. Within the subtypes of NSCLC, squamous cell carcinoma (SCC/NSCLC) represents approximately 30% of NSCLC. Systemic therapies used in the metastatic setting for SCC/NSCLC have shown limited benefit and are primarily aimed at prolonging survival and maintaining the quality of life for as long as possible, while minimizing side effects due to treatment. First line treatment for patients with SCC/NSCLC whose tumors do not express high levels of PD-L1 include a platinum-based chemotherapy doublet that does not contain pemetrexed, anti-VEGF antibody, or an anti-EGFR antibody necitumumab in combination with gemcitabine and cisplatin. Patients with at least 50% tumor cell staining for PD-L1 are offered first-line treatment with the anti-PD-1 inhibitor pembrolizumab. Patients who progress on an initial combination chemotherapy regimen may receive an anti-PD-1 or PD-L1 antibody, and combination chemotherapy is considered for patients whose disease has progressed after receiving PD-1/L1 inhibitors. New classes of therapy are urgently needed that can provide meaningful benefit to SCC/NSCLC patients.

The invention provides method for treating lung cancer with an anti-LIV1 antibody or antigen-binding fragment thereof or antibody-drug conjugate (e.g., a LIV1-ADC) described herein. In one aspect, the anti-LIV1 antibody or antigen-binding fragment thereof or antibody-drug conjugate (e.g., a LIV1-ADC) described herein are for use in a method of treating lung cancer in a subject. In some embodiments, the lung cancer is small cell lung cancer. In some embodiments, the subject has received prior systemic therapy for the small cell lung cancer. In some embodiments, the subject experienced disease progression on or after the prior systemic therapy for the small cell lung cancer. In some embodiments, the subject received prior therapy with a cytotoxic chemotherapy. In some embodiments, the subject received prior therapy with an inhibitor of PD-1 or PD-L1. In some embodiments, the subject received 1 line of systemic therapy for the small cell lung cancer. In some embodiments, the lung cancer is non-small cell lung cancer. In some embodiments, the non-small cell lung cancer is squamous cell carcinoma. In some embodiments, the non-small cell lung cancer has predominant squamous histology. In some embodiments, greater than 85% of the non-small cell lung cancer cells have squamous histology. In some embodiments, the non-small cell lung cancer is non-squamous cell carcinoma. In some embodiments, the subject received prior systemic therapy for the non-small cell lung cancer. In some embodiments, the subject experienced disease progression on or after the prior systemic therapy for the non-small cell lung cancer. In some embodiments, the subject received prior therapy with a cytotoxic chemotherapy. In some embodiments, the subject received prior therapy with a platinum-based therapy or platinum-based combination therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject received prior therapy with an inhibitor of PD-1 or PD-L1. In some embodiments, the inhibitor of PD-1 is selected from the group consisting of nivolumab (OPDIVO®, BMS-936558, MDX-1106), pembrolizumab (KEYTRUDA®, MK-3475), pidilizumab (CT-011) and cemiplimab (REGN2810). In some embodiments, the inhibitor of PD-L1 is selected from the group consisting of atezolizumab (TECENTRIQ®, MPDL3280A), avelumab (BAVENCIO®), durvalumab and BMS-936559. In some embodiments, the subject received 1 line of prior systemic therapy for the non-small cell lung cancer. In some embodiments, the lung cancer is an advanced stage cancer. In some embodiments, the advanced stage cancer is a stage 3 or 4 cancer. In some embodiments, the lung cancer is a recurrent cancer. In some embodiments, the subject received prior treatment with standard of care therapy for the cancer and failed the prior treatment. In a particular embodiment, the subject is a human.

B. Head and Neck Cancer

Head and neck cancers make up approximately 3% of cancers in the United States. Over 63,000 cases are estimated to have been diagnosed in 2017 and more than 13,000 patients died from this disease. Though human papilloma virus (HPV) infection also appears to contribute to head and neck cancers. More than 90-95% of oral and nasopharyngeal cancers are of squamous histology. Surgical resection, radiotherapy, and/or chemoradiation are frequently recommended for patients with early-stage or localized disease. Palliative chemotherapy, immunotherapy and/or supportive care are the most appropriate options for patients with locally recurrent or metastatic disease that are not amenable to definitive therapy. Platinum-based regimens are the preferred standard of care treatment for patients with recurrent or de novo metastatic squamous cell carcinoma of the head and neck (SCCHN). Cetuximab in combination with a platinum/5-FU regimen has demonstrated clinically meaningful benefits compared to platinum/5-FU alone. For patients progressing on first line treatment, second line treatment is with single agent chemotherapy, targeted therapy, or a checkpoint inhibitor such as nivolumab or pembrolizumab. Overall, there is a great unmet medical need for patients with SCCHN that have progressed after first line platinum combination therapy followed by second line PD-1 therapy.

The invention provides method for treating head and neck cancer with an anti-LIV1 antibody or antigen-binding fragment thereof or antibody-drug conjugate (e.g., a LIV1-ADC) described herein. In one aspect, the anti-LIV1 antibody or antigen-binding fragment thereof or antibody-drug conjugate (e.g., a LIV1-ADC) described herein are for use in a method of treating head and neck cancer in a subject. In some embodiments, the head and neck cancer is squamous cell carcinoma. In some embodiments, the head and neck cancer has predominant squamous histology. In some embodiments, greater than 85% of the head and neck cancer cells have squamous histology. In some embodiments, the subject received prior systemic therapy for the head and neck cancer. In some embodiments, the subject experienced disease progression on or after the prior systemic therapy for the head and neck cancer. In some embodiments, the subject received prior therapy with a cytotoxic chemotherapy. In some embodiments, the subject received prior therapy with a platinum-based therapy or platinum-based combination therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject received prior therapy with an inhibitor of PD-1 or PD-L1. In some embodiments, the inhibitor of PD-1 is selected from the group consisting of nivolumab (OPDIVO®, BMS-936558, MDX-1106), pembrolizumab (KEYTRUDA®, MK-3475), pidilizumab (CT-011) and cemiplimab (REGN2810). In some embodiments, the inhibitor of PD-L1 is selected from the group consisting of atezolizumab (TECENTRIQ®, MPDL3280A), avelumab (BAVENCIO®), durvalumab and BMS-936559. In some embodiments, the subject received 1 line of prior systemic therapy for the head and neck cancer. In some embodiments, the head and neck cancer is an advanced stage cancer. In some embodiments, the advanced stage cancer is a stage 3 or 4 cancer. In some embodiments, the head and neck cancer is a recurrent cancer. In some embodiments, the subject received prior treatment with standard of care therapy for the cancer and failed the prior treatment. In a particular embodiment, the subject is a human.

C. Esophageal Cancer

Esophageal cancer is the sixth leading cause of cancer-related mortality worldwide due to its overall poor prognosis. The global age-standardized incidence rate of esophageal squamous cell carcinoma (ESCC) is 1.4-13.6 per 100,000 people. Esophageal cancer is estimated to be responsible for 15,690 deaths and 16,940 new cases in the United States in 2016. The majority of patients present with locally advanced or systemic disease and outcomes remain poor despite advances in treatment. More effective treatments for these patients with locally advanced or systemic disease are urgently needed.

The invention provides method for treating esophageal carcinoma with an anti-LIV1 antibody or antigen-binding fragment thereof or antibody-drug conjugate (e.g., a LIV1-ADC) described herein. In one aspect, the anti-LIV1 antibody or antigen-binding fragment thereof or antibody-drug conjugate (e.g., a LIV1-ADC) described herein are for use in a method of treating esophageal carcinoma in a subject. In some embodiments, esophageal carcinoma is squamous cell carcinoma. In some embodiments, the esophageal carcinoma has predominant squamous histology. In some embodiments, greater than 85% of the esophageal cells have squamous histology. In some embodiments, the subject received prior systemic therapy for the esophageal cancer. In some embodiments, the subject experienced disease progression on or after the prior systemic therapy for the esophageal cancer. In some embodiments, the subject received prior therapy with a cytotoxic chemotherapy. In some embodiments, the subject received 1 line of prior systemic therapy for the esophageal cancer. In some embodiments, the esophageal carcinoma is an advanced stage cancer. In some embodiments, the advanced stage cancer is a stage 3 or 4 cancer. In some embodiments, the esophageal carcinoma is a recurrent cancer. In some embodiments, the subject received prior treatment with standard of care therapy for the cancer and failed the prior treatment. In a particular embodiment, the subject is a human.

D. Gastric and Gastroesophageal Junction Cancer

Gastric cancer, or stomach cancer, is most commonly caused by infection by the bacteria Helicobacter pylori. About 90 to 95% of cancers of the stomach are adenocarcinomas. Gastric cancer occurs mostly in adults (average age at diagnosis: 69 years). The incidence of gastric cancer is about 1 in 111. The overall 5-year relative survival rate of all people with gastric cancer in the United States is about 29%. Gastroesophageal junction adenocarcinoma is a cancer of the lower part of the esophagus. The incidence of gastroesophageal junction adenocarcinoma is rising rapidly in western countries, the treatment options are limited and the overall prognosis is extremely poor.

The invention provides method for treating gastric and gastroesophageal junction cancer with an anti-LIV1 antibody or antigen-binding fragment thereof or antibody-drug conjugate (e.g., a LIV1-ADC) described herein. In one aspect, the anti-LIV1 antibody or antigen-binding fragment thereof or antibody-drug conjugate (e.g., a LIV1-ADC) described herein are for use in a method of treating gastric or gastroesophageal junction cancer in a subject. In some embodiments, the solid tumor is gastric cancer. In some embodiments, the gastric cancer is gastric adenocarcinoma. In some embodiments, the subject received prior systemic therapy for the gastric cancer. In some embodiments, the subject experienced disease progression on or after the prior systemic therapy for the gastric cancer. In some embodiments, the subject received prior therapy with a cytotoxic chemotherapy. In some embodiments, the subject received prior therapy with a platinum-based therapy or platinum-based combination therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject overexpresses human epidermal growth factor receptor 2 (HER2). In some embodiments, the subject received prior HER2-targeted therapy. In some embodiments, the HER2-targeted therapy is selected from the group consisting of trastuzumab, pertuzumab, margetuximab, and nelipepimut-S. In some embodiments, the subject received 1 line of prior systemic therapy for the gastric cancer. In some embodiments, the solid tumor is gastroesophageal junction cancer. In some embodiments, the gastroesophageal junction cancer is gastroesophageal junction adenocarcinoma. In some embodiments, the subject received prior systemic therapy for the gastroesophageal junction cancer. In some embodiments, the subject experienced disease progression on or after the prior systemic therapy for the gastroesophageal junction cancer. In some embodiments, the subject received prior therapy with a cytotoxic chemotherapy. In some embodiments, the subject received prior therapy with a platinum-based therapy or platinum-based combination therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject overexpresses human epidermal growth factor receptor 2 (HER2). In some embodiments, the subject received prior HER2-targeted therapy. In some embodiments, the HER2-targeted therapy is selected from the group consisting of trastuzumab, pertuzumab, margetuximab, and nelipepimut-S. In some embodiments, the subject received 1 line of prior systemic therapy for the gastroesophageal junction cancer. In some embodiments, the gastric or gastroesophageal cancer is an advanced stage cancer. In some embodiments, the advanced stage cancer is a stage 3 or 4 cancer. In some embodiments, the gastric or gastroesophageal carcinoma is a recurrent cancer. In some embodiments, the subject received prior treatment with standard of care therapy for the cancer and failed the prior treatment. In a particular embodiment, the subject is a human.

E. Breast Cancer

Breast cancers are classified on the basis of three protein expression markers: estrogen receptor (ER), progesterone receptor (PgR), and the overexpression of the growth factor receptor HER2/neu. Hormonal therapies, including tamoxifen and aromatase inhibitors, can be effective in treating tumors that express the hormone receptors ER and PgR. HER2-directed therapies are useful for tumors that express HER2/neu; these tumors are the only class of breast cancer that is currently eligible for immunotherapy. For these patients, unconjugated antibodies, such as Herceptin or Perjeta, are generally used in combination with chemotherapy.

The invention provides methods of treating cancers, such as breast cancer, with antibodies and antigen-binding fragments thereof and antibody-drug conjugates. In some embodiments, the invention provides methods of treating cancers, such as breast cancer, with antibody-drug conjugates. In some embodiments, the antibody-drug conjugate comprises an antibody conjugated to an auristatin. In some embodiments, the auristatin is a monomethyl auristatin. In some embodiments, the monomethyl auristatin is monomethyl auristatin E. In one aspect, the invention provides methods of treating disorders associated with cells that express LIV-1, e.g., cancers (e.g., breast cancers such as locally advanced breast cancer or metastatic breast cancer). As a result, the invention provides a method of treating a subject, for example, a subject with breast cancer, using the anti-LIV1 antibodies and antigen-binding fragments thereof and antibody-drug conjugates described herein. The method comprises administering an effective amount of an anti-LIV1 antibody or a composition comprising an anti-LIV1 antibody or an antigen-binding fragment thereof or an antibody-drug conjugate (e.g., a LIV1-ADC) to a subject in need thereof. In some embodiments, the cancer is an advanced stage cancer. In some embodiments, the advanced stage cancer is metastatic cancer. In some embodiments, the cancer is unresectable. In some embodiments, the cancer is locally advanced. In some embodiments, the cancer is recurrent cancer. In some embodiments, the subject received prior treatment with standard of care therapy for the cancer and failed the prior treatment. In some embodiments, the subject has been previously treated with one or more therapeutic agents and did not respond to the treatment, wherein the one or more therapeutic agents is not an antibody-drug conjugate (e.g., LIV1-ADC). In some embodiments, the subject has been previously treated with one or more therapeutic agents and relapsed after the treatment, wherein the one or more therapeutic agents is not an antibody-drug conjugate (e.g., LIV1-ADC). In some embodiments, the subject has been previously treated with one or more therapeutic agents and has experienced disease progression during treatment, wherein the one or more therapeutic agents is an antibody-drug conjugate (e.g., LIV1-ADC). In some embodiments, the subject is a human.

Exemplary breast cancers are those that express LIV1 in a cell expressing the cancer (i.e., LIV1-expressing cancers). In certain exemplary embodiments, a breast cancer is selected from the group consisting of carcinomas, sarcomas, phyllodes, Paget disease, and angiosarcomas. The breast cancer may be in situ (e.g., ductal carcinoma in situ (DCIS), lobular carcinoma in situ (LCIS) and the like) or invasive/infiltrating (e.g., invasive ductal carcinoma (IDC), invasive lobular carcinoma (IIC), inflammatory breast cancer (IBC) and the like).

Breast cancer may have the following characteristics: estrogen receptor positive (ER+); estrogen receptor positive (ER−); progesterone receptor positive (PR+); progesterone receptor negative (PR−); hormone receptor positive (HR+); hormone receptor negative (HR−); HER2 gene overexpressing (HER2+); HER2 gene wild-type or under-expressing (HER2−); group 1 (luminal A), i.e., ER+/PR+/HER2−; group 2 (luminal B), i.e., ER+/PR−/HER2+; group 3 (HER2+), i.e., ER−/PR−/HER2+; and group 4 (basal-like or triple negative (TN)), i.e., ER−/PR−/HER2−.

A breast cancer can further be categorized as grade 1, 2 or 3. Grade 1 or well-differentiated (score 3, 4, or 5) breast cancer comprises cells that are slower-growing, and look more like normal breast tissue than the higher grades of breast cancer. Grade 2 or moderately differentiated (score 6, 7) breast cancer has cells that grow at a speed of and look like cells somewhere between grades 1 and 3. Grade 3 or poorly differentiated (score 8, 9) breast cancer has cells that look very different from normal cells and typically grow and spread faster than grades 1 or 2.

In certain exemplary embodiments, a breast cancer is an incurable, unresectable, locally advanced or metastatic breast cancer (LA/MBC). In certain embodiments, a breast cancer is either a triple negative (TN) (ER−/PR−/HER2−) breast cancer, an ER- and/or PR+/HER2− breast cancer, and an LA/MBC breast cancer. In certain exemplary embodiments, the breast cancer is HER2+ and LA/MBC. In certain exemplary embodiments, a breast cancer is TN and LA/MBC. In certain exemplary embodiments, a breast cancer is selected from the group consisting of a TN breast cancer, a metastatic breast cancer, and a metastatic, TN breast cancer.

In certain exemplary embodiments, the present invention provides a method for treating breast cancer in a human. In some embodiments, the present invention provides a method for treating ER+ breast cancer in a subject. In some embodiments, the subject with ER+ breast cancer is not a candidate for hormonal therapy. In some embodiments, the subject with ER+ breast cancer received one prior cytotoxic regimen. In some embodiments, the subject with ER+ breast cancer received two or more prior cytotoxic regimens. In some embodiments, the present invention provides a method for treating ER+/HER2− breast cancer in a subject. In some embodiments, the subject with ER+/HER2− breast cancer is not a candidate for hormonal therapy. In some embodiments, the subject with ER+/HER2− breast cancer has not received a prior cytotoxic regimen. In some embodiments, the subject with ER+/HER2− breast cancer received one prior cytotoxic regimen. In some embodiments, the subject with ER+/HER2− breast cancer received two or more prior cytotoxic regimens. In some embodiments, the present invention provides a method for treating PR+/HER2− breast cancer in a subject. In some embodiments, the subject with PR+/HER2− breast cancer is not a candidate for hormonal therapy. In some embodiments, the subject with PR+/HER2− breast cancer received one prior cytotoxic regimen. In some embodiments, the subject with PR+/HER2− breast cancer received two or more prior cytotoxic regimens. In some embodiments, the present invention provides a method of treating ER+/PR+HER2− breast cancer in a subject. In some embodiments, the subject with ER+/PR+/HER2− breast cancer is not a candidate for hormonal therapy. In some embodiments, the subject with ER+/PR+HER2− breast cancer received one prior cytotoxic regimen. In some embodiments, the subject with ER+/PR+HER2− breast cancer received two or more prior cytotoxic regimens. In some embodiments, the present invention provides a method of treating triple negative breast cancer in a subject. In some embodiments, the subject with triple negative breast cancer received one non-hormonally directed prior therapy. In some embodiments, the subject with triple negative breast cancer received one prior cytotoxic regimen. In some embodiments, the subject with triple negative breast cancer received two or more prior cytotoxic regimens. In some embodiments, the present invention provides a method of treating HR+ breast cancer in a subject. In some embodiments, the subject with HR+ breast cancer received one prior cytotoxic regimen. In some embodiments, the subject with HR+ breast cancer received two or more prior cytotoxic regimens. In some embodiments, the present invention provides a method of treating HR+/ER+/HER2− breast cancer in a subject. In some embodiments, the subject with HR+/ER+/HER2− breast cancer is not a candidate for hormonal therapy. In some embodiments, the subject with HR+/ER+/HER2− breast cancer is eligible for chemotherapy. In some embodiments, the subject with HR+/ER+/HER2− breast cancer received one prior cytotoxic regimen. In some embodiments, the subject with HR+/ER+/HER2− breast cancer received one prior non-hormonally-directed therapy regimen. In some embodiments, the present invention provides a method of treating HR+/PR+/HER2− breast cancer in a subject. In some embodiments, the subject with HR+/PR+/HER2− breast cancer is not a candidate for hormonal therapy. In some embodiments, the subject with HR+/PR+/HER2− breast cancer is eligible for chemotherapy. In some embodiments, the subject with HR+/PR+/HER2− breast cancer received one prior cytotoxic regimen. In some embodiments, the subject with HR+/PR+/HER2− breast cancer received one prior non-hormonally-directed therapy regimen. In some embodiments, the present invention provides a method of treating HR+/ER+/PR+/HER2− breast cancer in a subject. In some embodiments, the subject with HR+/ER+/PR+/HER2− breast cancer is not a candidate for hormonal therapy. In some embodiments, the subject with HR+/ER+/PR+/HER2− breast cancer is eligible for chemotherapy. In some embodiments, the subject with HR+/ER+/PR+/HER2− breast cancer received one prior cytotoxic regimen. In some embodiments, the subject with HR+/ER+/PR+HER2− breast cancer received one prior non-hormonally-directed therapy regimen. In some embodiments, the present invention provides a method of treating HER2+ breast cancer in a subject. In some embodiments, the subject with HER2+ breast cancer received one prior cytotoxic regimen. In some embodiments, the subject with HER2+ breast cancer received two or more prior cytotoxic regimens. In some embodiments, the present invention provides a method of treating HR+/HER2+ breast cancer in a subject. In some embodiments, the subject with HR+/HER2+ breast cancer is eligible for chemotherapy. In some embodiments, the subject with HR+/HER2+ breast cancer is not eligible for chemotherapy. In some embodiments, the subject with HR+/HER2+ breast cancer is not a candidate for hormonal therapy. In some embodiments, the breast cancer is an advanced breast stage cancer. In some embodiments, the advanced stage breast cancer is metastatic breast cancer. In some embodiments, the breast cancer is unresectable. In some embodiments, the breast cancer is locally advanced. In some embodiments, the breast cancer is recurrent breast cancer. In some embodiments, the subject received prior treatment with standard of care therapy for the breast cancer and failed the prior treatment. In some embodiments, the subject has been previously treated with one or more therapeutic agents and did not respond to the treatment, wherein the one or more therapeutic agents is not an antibody-drug conjugate (e.g., LIV1-ADC). In some embodiments, the subject has been previously treated with one or more therapeutic agents and relapsed after the treatment, wherein the one or more therapeutic agents is not an antibody-drug conjugate (e.g., LIV1-ADC). In some embodiments, the subject has been previously treated with one or more therapeutic agents and has experienced disease progression during treatment, wherein the one or more therapeutic agents is an antibody-drug conjugate (e.g., LIV1-ADC). In some embodiments, the subject is a human.

F. Adverse Events

In one aspect, a method of treating cancer, such as solid tumors, e.g., locally advanced or metastatic solid tumors (e.g., small cell lung cancer, non-small cell lung cancer, head and neck squamous cell carcinoma, esophageal squamous cell carcinoma, and gastric and gastroesophageal junction adenocarcinoma) and breast cancer, with an antibody or antigen-binding fragment thereof or antibody-drug conjugate described herein (e.g., LIV1-ADC), results in the subject developing one or more adverse events. In some embodiments, the subject is administered an additional therapeutic agent to eliminate or reduce the severity of the adverse event. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some of any of the embodiments herein, the subject is administered a treatment with the additional therapeutic agent to eliminate or reduce the severity of the adverse. In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

In one aspect, a subject treated with an antibody or antigen-binding fragment thereof or antibody-drug conjugate described herein (e.g., LIV1-ADC), is at risk of developing one or more adverse events. In some embodiments, the subject is administered an additional therapeutic agent to prevent the development of the adverse event or to reduce the severity of the adverse event. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some of any of the embodiments herein, the subject is administered a treatment with the additional therapeutic agent to prevent the development of the adverse event or to reduce the severity of the adverse event. In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

G. Treatment Outcomes

In one aspect, a method of treating a solid tumor, e.g., small cell lung cancer, non-small cell lung cancer, head and neck squamous cell carcinoma, esophageal squamous cell carcinoma, and gastric and gastroesophageal junction adenocarcinoma or breast cancer with an antibody or antigen-binding fragment thereof or antibody-drug conjugate (e.g., LIV1-ADC) as described herein results in an improvement in one or more therapeutic effects in the subject after administration of the antibody or antigen-binding fragment thereof or antibody-drug conjugate (e.g., LIV1-ADC) relative to a baseline. In some embodiments, the one or more therapeutic effects is the size of the tumor derived from the cancer (e.g., small cell lung cancer, non-small cell lung cancer, head and neck squamous cell carcinoma, esophageal squamous cell carcinoma, gastric and gastroesophageal junction adenocarcinoma, or breast cancer), the objective response rate, the duration of response, the time to response, progression free survival, overall survival, or any combination thereof. In one embodiment, the one or more therapeutic effects is the size of the tumor derived from the cancer (e.g., small cell lung cancer, non-small cell lung cancer, head and neck squamous cell carcinoma, esophageal squamous cell carcinoma, gastric and gastroesophageal junction adenocarcinoma, or breast cancer). In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an antibody or antigen-binding fragment thereof or antibody-drug conjugate as described herein, such as e.g., a LIV1-ADC, may include the following criteria (RECIST Criteria 1.1):

| | Category | Criteria |
|---|---|---|
| Based on | Complete | Disappearance of all target lesions. Any pathological |
| target | Response (CR) | lymph nodes must have reduction in short axis to <10 mm. |
| lesions | Partial | ≥30% decrease in the sum of the longest diameter (LD) |
| | Response (PR) | of target lesions, taking as reference the baseline sum of LDs. |
| | Stable | Neither sufficient shrinkage to qualify for PR nor |
| | Disease (SD) | sufficient increase to qualify for PD, taking as |
| | | reference the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on | CR | Disappearance of all non-target lesions and |
| non-target | | normalization of tumor marker level. All lymph nodes |
| lesions | | must be non-pathological in size (<10 mm short axis). |
| | SD | Persistence of one or more non-target lesion(s) or/and |
| | | maintenance of tumor marker level above the normal limits. |
| | PD | Appearance of one or more new lesions and/or |
| | | unequivocal progression of existing non-target lesions. |

In one embodiment of the methods or uses or product for uses provided herein, the effectiveness of treatment with an antibody or antigen-binding fragment thereof or antibody-drug conjugate as described herein, such as e.g., a LIV1-ADC, is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an antibody or antigen-binding fragment thereof or antibody-drug conjugate as described herein, such as e.g., a LIV1-ADC, is assessed by measuring the size of a tumor derived from the cancer (e.g., small cell lung cancer, non-small cell lung cancer, head and neck squamous cell carcinoma, esophageal squamous cell carcinoma, gastric and gastroesophageal junction adenocarcinoma, or breast cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antigen-binding fragment thereof or antibody-drug conjugate described herein (e.g., a LIV1-ADC). In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antigen-binding fragment thereof or antibody-drug conjugate described herein (e.g., a LIV1-ADC). In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by ultrasound.

In one embodiment of the methods or uses or product for uses provided described herein, response to treatment with an antibody or antigen-binding fragment thereof or antibody-drug conjugate described herein (e.g., a LIV1-ADC), promotes regression of a tumor derived from the cancer (e.g., small cell lung cancer, non-small cell lung cancer, head and neck squamous cell carcinoma, esophageal squamous cell carcinoma, gastric and gastroesophageal junction adenocarcinoma, or breast cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antigen-binding fragment thereof or antibody-drug conjugate described herein (e.g., a LIV1-ADC). In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antigen-binding fragment thereof or antibody-drug conjugate described herein (e.g., a LIV1-ADC). In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In one embodiment, regression of a tumor is determined by measuring the size of the tumor by positron emission tomography (PET). In one embodiment, regression of a tumor is determined by measuring the size of the tumor by ultrasound.

In one embodiment of the methods or uses or product for uses described herein, response to treatment with an antibody or antigen-binding fragment thereof or antibody-drug conjugate described herein (e.g., a LIV1-ADC), is assessed by measuring the time of progression free survival after administration of the antibody or antigen-binding fragment thereof or antibody-drug conjugate described herein (e.g., a LIV1-ADC). In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the subject exhibits progression-free survival of at least about 6 months after administration of the antibody or antigen-binding fragment thereof or antibody-drug conjugate described herein (e.g., a LIV1-ADC). In some embodiments, the subject exhibits progression-free survival of at least about one year after administration of the antibody or antigen-binding fragment thereof or antibody-drug conjugate described herein (e.g., a LIV1-ADC). In some embodiments, the subject exhibits progression-free survival of at least about two years after administration of the antibody or antigen-binding fragment thereof or antibody-drug conjugate described herein (e.g., a LIV1-ADC). In some embodiments, the subject exhibits progression-free survival of at least about three years after administration of the antibody or antigen-binding fragment thereof or antibody-drug conjugate described herein (e.g., a LIV1-ADC). In some embodiments, the subject exhibits progression-free survival of at least about four years after administration of the antibody or antigen-binding fragment thereof or antibody-drug conjugate described herein (e.g., a LIV1-ADC). In some embodiments, the subject exhibits progression-free survival of at least about five years after administration of the antibody or antigen-binding fragment thereof or antibody-drug conjugate described herein (e.g., a LIV1-ADC). In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the subject exhibits progression-free survival of at least 6 months after administration of the antibody or antigen-binding fragment thereof or antibody-drug conjugate described herein (e.g., a LIV1-ADC). In some embodiments, the subject exhibits progression-free survival of at least one year after administration of the antibody or antigen-binding fragment thereof or antibody-drug conjugate described herein (e.g., a LIV1-ADC). In some embodiments, the subject exhibits progression-free survival of at least two years after administration of the antibody or antigen-binding fragment thereof or antibody-drug conjugate described herein (e.g., a LIV1-ADC). In some embodiments, the subject exhibits progression-free survival of at least three years after administration of the antibody or antigen-binding fragment thereof or antibody-drug conjugate described herein (e.g., a LIV1-ADC). In some embodiments, the subject exhibits progression-free survival of at least four years after administration of the antibody or antigen-binding fragment thereof or antibody-drug conjugate described herein (e.g., a LIV1-ADC). In some embodiments, the subject exhibits progression-free survival of at least five years after administration of the antibody or antigen-binding fragment thereof or antibody-drug conjugate described herein (e.g., a LIV1-ADC).

In one embodiment of the methods or uses or product for uses described herein, response to treatment with an antibody or antigen-binding fragment thereof or antibody-drug conjugate described herein (e.g., a LIV1-ADC), is assessed by measuring the time of overall survival after administration of the antibody or antigen-binding fragment thereof or antibody-drug conjugate described herein (e.g., a LIV1-ADC). In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the subject exhibits overall survival of at least about 6 months after administration of the antibody or antigen-binding fragment thereof or antibody-drug conjugate described herein (e.g., a LIV1-ADC). In some embodiments, the subject exhibits overall survival of at least about one year after administration of the antibody or antigen-binding fragment thereof or antibody-drug conjugate described herein (e.g., a LIV1-ADC). In some embodiments, the subject exhibits overall survival of at least about two years after administration of the antibody or antigen-binding fragment thereof or antibody-drug conjugate described herein (e.g., a LIV1-ADC). In some embodiments, the subject exhibits overall survival of at least about three years after administration of the antibody or antigen-binding fragment thereof or antibody-drug conjugate described herein (e.g., a LIV1-ADC). In some embodiments, the subject exhibits overall survival of at least about four years after administration of the antibody or antigen-binding fragment thereof or antibody-drug conjugate described herein (e.g., a LIV1-ADC). In some embodiments, the subject exhibits overall survival of at least about five years after administration of the antibody or antigen-binding fragment thereof or antibody-drug conjugate described herein (e.g., a LIV1-ADC). In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the subject exhibits overall survival of at least 6 months after administration of the antibody or antigen-binding fragment thereof or antibody-drug conjugate described herein (e.g., a LIV1-ADC). In some embodiments, the subject exhibits overall survival of at least one year after administration of the antibody or antigen-binding fragment thereof or antibody-drug conjugate described herein (e.g., a LIV1-ADC). In some embodiments, the subject exhibits overall survival of at least two years after administration of the antibody or antigen-binding fragment or antibody-drug conjugate thereof described herein (e.g., a LIV1-ADC). In some embodiments, the subject exhibits overall survival of at least three years after administration of the antibody or antigen-binding fragment thereof or antibody-drug conjugate described herein (e.g., a LIV1-ADC). In some embodiments, the subject exhibits overall survival of at least four years after administration of the antibody or antigen-binding fragment thereof or antibody-drug conjugate described herein (e.g., a LIV1-ADC). In some embodiments, the subject exhibits overall survival of at least five years after administration of the antibody or antigen-binding fragment thereof described herein (e.g., a LIV1-ADC).

In one embodiment of the methods or uses or product for uses described herein, response to treatment with an antibody or antigen-binding fragment thereof or antibody-drug conjugate described herein (e.g., a LIV1-ADC), is assessed by measuring the duration of response to the antibody or antigen-binding fragment thereof or antibody-drug conjugate described herein (e.g., a LIV1-ADC) after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about 6 months after administration of the antibody or antigen-binding fragment thereof or antibody-drug conjugate described herein (e.g., a LIV1-ADC). In some embodiments, the duration of response to anti-LIV1 antibody or antigen-binding fragment thereof or antibody-drug conjugate described herein (e.g., a LIV1-ADC) is at least about one year after administration of the anti-LIV1 antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about two years after administration of the antibody or antigen-binding fragment thereof or antibody-drug conjugate described herein (e.g., a LIV1-ADC). In some embodiments, the duration of response to the anti-LIV1 antibody or antigen-binding fragment thereof or antibody-drug conjugate described herein (e.g., a LIV1-ADC) is at least about three years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about four years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about five years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least 6 months after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least one year after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least two years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least three years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least four years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least five years after administration of the antibody or antigen-binding fragment thereof or antibody-drug conjugate described herein (e.g., a LIV1-ADC).

### IV. Pharmaceutical Compositions and Formulations

For therapeutic use, an antibody or antigen-binding fragment thereof or antibody-drug conjugate (e.g., a LIV1-ADC) is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, antibody or antigen-binding fragment thereof or antibody-drug conjugate (e.g., a LIV1-ADC) compositions of the present invention can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

Suitable pharmaceutical excipients and/or additives for use in the antibody molecule compositions according to the invention are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998).

Pharmaceutical compositions containing an antibody or antigen-binding fragment thereof or antibody-drug conjugate (e.g., a LIV1-ADC) as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences (1990) supra. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

The compositions of this invention may be in a variety of forms. These include, for example, liquid, semi-solid and solid dosage forms, such as liquid solutions (e.g., injectable and infusible solutions), dispersions or suspensions, and liposomes. The particular form depends on the intended mode of administration and therapeutic application. In exemplary embodiments, compositions provided are in the form of injectable or infusible solutions. Exemplary administration is parenteral (e.g., intravenous, subcutaneous, intraocular, intraperitoneal, intramuscular). In an exemplary embodiment, the preparation is administered by intravenous infusion or injection. In another preferred embodiment, the preparation is administered by intramuscular or subcutaneous injection.

The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

Exemplary dosages of an antibody or antigen-binding fragment thereof or antibody-drug conjugate (e.g., a LIV1-ADC) are about 0.5 mg/kg of a subject's body weight, about 0.75 mg/kg of a subject's body weight, about 1.0 mg/kg of a subject's body weight, about 1.25 mg/kg of a subject's body weight, about 1.5 mg/kg of a subject's body weight, about 1.67 mg/kg of a subject's body weight, about 1.75 mg/kg of a subject's body weight, about 2.0 mg/kg of a subject's body weight, about 2.25 mg/kg of a subject's body weight, about 2.5 mg/kg of a subject's body weight, about 2.75 mg/kg of a subject's body weight, or about 2.8 mg/kg of a subject's body weight. In a particular embodiment, an exemplary dose of LIV1-ADC is about 0.75 mg/kg of a subject's body weight. In another particular embodiment, an exemplary dose of LIV1-ADC is about 1.0 mg/kg of a subject's body weight. In another particular embodiment, an exemplary dose of LIV1-ADC is about 1.25 mg/kg of a subject's body weight. In another particular embodiment, an exemplary dose of LIV1-ADC is about 1.5 mg/kg of a subject's body weight. In another particular embodiment, an exemplary dose of LIV1-ADC is about 1.67 mg/kg of a subject's body weight. In another particular embodiment, an exemplary dose of LIV1-ADC is about 1.75 mg/kg of a subject's body weight. In another particular embodiment, an exemplary dose of LIV1-ADC is about 2.0 mg/kg of a subject's body weight. In another particular embodiment, an exemplary dose of LIV1-ADC is about 2.25 mg/kg of a subject's body weight. In another particular embodiment, an exemplary dose of LIV1-ADC is about 2.5 mg/kg of a subject's body weight. In another particular embodiment, an exemplary dose of LIV1-ADC is about 2.75 mg/kg of a subject's body weight. In another particular embodiment, an exemplary dose of LIV1-ADC is about 2.8 mg/kg of a subject's body weight. In another particular embodiment, a maximum exemplary dose of LIV1-ADC is about 100 mg per cycle. In another particular embodiment, a maximum exemplary dose of LIV1-ADC is about 125 mg per cycle. In another particular embodiment, a maximum exemplary dose of LIV1-ADC is about 200 mg per cycle. In another particular embodiment, a maximum exemplary dose of LIV1-ADC is about 250 mg per cycle.

In certain exemplary embodiments, a subject is administered a dose of about 2.5 mg/kg, at a maximum dose of about 200 mg, once every three weeks. In certain exemplary embodiments, a subject is administered an intravenous dose of about 2.5 mg/kg, at a maximum dose of about 200 mg, once every three weeks.

In certain exemplary embodiments, a subject is administered a dose of about 2.5 mg/kg, at a maximum dose of about 250 mg, once every three weeks. In certain exemplary embodiments, a subject is administered an intravenous dose of about 2.5 mg/kg, at a maximum dose of about 250 mg, once every three weeks.

In certain exemplary embodiments, a subject is administered a dose of about 1.0 mg/kg, at a maximum dose of about 100 mg, once every one week. In certain exemplary embodiments, a subject is administered an intravenous dose of about 1.0 mg/kg, at a maximum dose of about 100 mg, once every one week.

In certain exemplary embodiments, a subject is administered a dose of about 1.25 mg/kg, at a maximum dose of about 125 mg, once every one week. In certain exemplary embodiments, a subject is administered an intravenous dose of about 1.25 mg/kg, at a maximum dose of about 125 mg, once every one week.

In certain exemplary embodiments, a subject is administered a dose of about 2.5 mg/kg, at a maximum dose of about 250 mg, once every three weeks. In certain exemplary embodiments, a subject is administered an intravenous dose of about 2.5 mg/kg, at a maximum dose of about 250 mg, once every three weeks. In certain exemplary embodiments, the subject is further administered GCSF. In certain exemplary embodiments, if the anti-LIV1 antibody or antigen-binding fragment thereof (e.g., a LIV1-ADC) is used at a dose of greater than or equal to about 200 mg and less than or equal to about 250 mg once every three weeks, the subject is further administered GCSF. In certain exemplary embodiments, if the anti-LIV1 antibody or antigen-binding fragment thereof (e.g., a LIV1-ADC) is used at a dose of greater than or equal to 200 mg and less than or equal to 250 mg once every three weeks, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In certain exemplary embodiments, a subject is administered a dose of about 1.0 mg/kg, at a maximum dose of about 100 mg, once every one week. In certain exemplary embodiments, a subject is administered an intravenous dose of about 1.0 mg/kg, at a maximum dose of about 100 mg, once every one week. In certain exemplary embodiments, the subject is further administered GCSF. In certain exemplary embodiments, if the anti-LIV1 antibody or antigen-binding fragment thereof (e.g., a LIV1-ADC) is used at a dose of greater than or equal to about 80 mg and less than or equal to about 100 mg once every one week, the subject is further administered GCSF. In certain exemplary embodiments, if the anti-LIV1 antibody or antigen-binding fragment thereof (e.g., a LIV1-ADC) is used at a dose of greater than or equal to 80 mg and less than or equal to 100 mg once every one week, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In certain exemplary embodiments, a subject is administered a dose of about 1.25 mg/kg, at a maximum dose of about 125 mg, once every one week. In certain exemplary embodiments, a subject is administered an intravenous dose of about 1.25 mg/kg, at a maximum dose of about 125 mg, once every one week. In certain exemplary embodiments, the subject is further administered GCSF. In certain exemplary embodiments, if the anti-LIV1 antibody or antigen-binding fragment thereof (e.g., a LIV1-ADC) is used at a dose of greater than or equal to about 100 mg and less than or equal to about 125 mg once every one week, the subject is further administered GCSF. In certain exemplary embodiments, if the anti-LIV1 antibody or antigen-binding fragment thereof (e.g., a LIV1-ADC) is used at a dose of greater than or equal to 100 mg and less than or equal to 125 mg once every one week, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

In certain exemplary embodiments, a subject is administered LIV1-ADC at a dose of about 0.75 mg/kg of body weight once every 7 days (±1 day). In certain exemplary embodiments, a subject is administered LIV1-ADC at a dose of about 1.0 mg/kg of body weight once every 7 days (±1 day). In certain exemplary embodiments, a subject is administered LIV1-ADC at a dose of about 1.25 mg/kg of body weight once every 7 days (±1 day). In certain exemplary embodiments, a subject is administered LIV1-ADC at a dose of about 1.5 mg/kg of body weight once every 7 days (±1 day). In certain exemplary embodiments, a subject is administered LIV1-ADC at a dose of about 1.67 mg/kg of body weight once every 7 days (±1 day). In certain exemplary embodiments, a subject is administered LIV1-ADC at a dose of about 1.25 mg/kg of body weight on day 1 and day 8 of a three week treatment cycle. In certain exemplary embodiments, a subject is administered LIV1-ADC at a dose of about 1.5 mg/kg of body weight on day 1 and day 8 of a three week treatment cycle. In certain exemplary embodiments, a subject is administered LIV1-ADC at a dose of about 1.75 mg/kg of body weight on day 1 and day 8 of a three week treatment cycle. In certain exemplary embodiments, a subject is administered LIV1-ADC at a dose of about 2.0 mg/kg of body weight on day 1 and day 8 of a three week treatment cycle. In certain exemplary embodiments, a subject is administered LIV1-ADC at a dose of about 2.25 mg/kg of body weight on day 1 and day 8 of a three week treatment cycle. In certain exemplary embodiments, a subject is administered LIV1-ADC at a dose of about 2.5 mg/kg of body weight on day 1 and day 8 of a three week treatment cycle.

In certain exemplary embodiments, the subject is further administered GCSF. In certain exemplary embodiments, if the anti-LIV1 antibody or antigen-binding fragment thereof or antibody-drug conjugate (e.g., a LIV1-ADC) is used at a dose of greater than or equal to about 200 mg and less than or equal to about 250 mg, the subject is further administered GCSF. In certain exemplary embodiments, if the anti-LIV1 antibody or antigen-binding fragment thereof or antibody-drug conjugate (e.g., a LIV1-ADC) is used at a dose of greater than or equal to 200 mg and less than or equal to 250 mg, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

The present invention provides a kit, comprising packaging material and at least one vial comprising a solution of at least one an antibody or antigen-binding fragment thereof or antibody-drug conjugate (e.g., a LIV1-ADC) with the prescribed buffers and/or preservatives, optionally in an aqueous diluent. The concentration of preservative used in the formulation is a concentration sufficient to yield an anti-microbial effect. Such concentrations are dependent on the preservative selected and are readily determined by the skilled artisan.

Various delivery systems can be used to administer antibodies or antigen-binding fragments thereof or antibody-drug conjugate to a subject. In certain exemplary embodiments, administration of an antibody or antigen-binding fragment thereof or antibody-drug conjugate (e.g., a LIV1-ADC) is by intravenous infusion.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized, it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present invention can be used with the methods described herein or with other methods for treating disease. The antibody or antigen-binding fragment thereof or antibody-drug conjugate (e.g., LIV1-ADC) formulations may be further diluted before administration to a subject. In some embodiments, the formulations will be diluted with saline and held in IV bags or syringes before administration to a subject. Accordingly, in some embodiments, the methods for treating a cancer, such as a LIV1-expressing cancer, in a subject will comprise administering to a subject in need thereof a weekly dose of a pharmaceutical composition comprising an antibody or antigen-binding fragment thereof or antibody-drug conjugate (e.g., a LIV1-ADC).

### V. Articles of Manufacture and Kits

In another aspect, an article of manufacture or kit is provided which comprises an antibody or antigen-binding fragment thereof or antibody-drug conjugate described herein (e.g., a LIV1-ADC). The article of manufacture or kit may further comprise instructions for use of the antibody or antigen-binding fragment thereof or antibody-drug conjugate described herein (e.g., a LIV1-ADC) in the methods of the invention. Thus, in certain embodiments, the article of manufacture or kit comprises instructions for the use of an anti-LIV1 antibody or antigen-binding fragment thereof described herein (e.g., a LIV1-ADC) in methods for treating cancer (e.g., breast cancer) in a subject comprising administering to the subject an effective amount of an anti-LIV1 antibody or antigen-binding fragment thereof or antibody-drug conjugate described herein (e.g., a LIV1-ADC). In some embodiments the cancer is a locally advanced cancer. In some embodiments, the cancer is a metastatic cancer. In some embodiments, the cancer is breast cancer as described herein. In certain embodiments, the article of manufacture or kit comprises instructions for the use of an anti-LIV1 antibody or antigen-binding fragment thereof described herein (e.g., a LIV1-ADC) in methods for treating cancer (e.g., locally advanced or metastatic solid tumors (e.g., small cell lung cancer, non-small cell lung cancer, head and neck squamous cell carcinoma, esophageal squamous cell carcinoma, and gastric and gastroesophageal junction adenocarcinoma)) in a subject comprising administering to the subject an effective amount of an anti-LIV1 antibody or antigen-binding fragment thereof described herein (e.g., a LIV1-ADC). In some embodiments the cancer is a locally advanced solid tumor. In some embodiments, the cancer is a metastatic solid tumor. In some embodiments, the cancer is small cell lung cancer as described herein. In some embodiments, the cancer is non-small cell lung cancer as described herein. In some embodiments, the cancer is head and neck cancer as described herein. In some embodiments, the cancer is esophageal carcinoma as described herein. In some embodiments, the cancer is gastric cancer as described herein. In some embodiments, the cancer is gastroesophageal junction cancer as described herein. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer, e.g., breast cancer, as described herein in a subject. The label or package insert may further indicate that the formulation is useful or intended for subcutaneous, intravenous (e.g., intravenous infusion), or other modes of administration for treating lung cancer, head and neck cancer, esophageal cancer, gastric cancer, or gastroesophageal junction cancer as described herein in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein an anti-LIV1 antibody or antigen-binding fragment thereof or antibody-drug conjugate described herein (e.g., a LIV1-ADC) is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

The article of manufacture or kit herein optionally further comprises a container comprising a second medicament, wherein the second medicament is for eliminating or reducing the severity of one or more adverse events, wherein an antibody or antigen-binding fragment thereof or antibody-drug conjugate described herein (e.g., a LIV1-ADC) is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

In some embodiments, an antibody or antigen-binding fragment thereof or antibody-drug conjugate described herein (e.g., a LIV1-ADC) is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Example 1: Dose Evaluation in Expansion and Escalation in Fractionated Dosing Scheme

Ladiratuzumab vedotin (LV) is an antibody-drug conjugate comprising a LIV1 targeted human monoclonal immunoglobulin conjugated via a protease-cleavable valine citrulline linker to the drug monomethyl auristatin E (MMAE), a dolastatin 10 analog. Dolastatins and auristatins belong to a class of chemotherapies that act as microtubule disrupting agents.

Human patients with breast cancer expressing LIV1 will be treated with ladiratuzumab vedotin (LV).

Previously, LV was administered by intravenous (IV) infusion at a dose of 2.5 mg/kg on Day 1 of each 21-day cycle (Q3W). To investigate whether smaller fractionated dose can result in desirable ADC pharmacokinetics, weekly dosing (Q1W), or dosing on Day 1, Day 8 but with “drug holiday” on the 3rd week (D1,8-Q3W) will be investigated. Increasing the frequency of ADC dosing, such as by the use of Q1W dosing, may result in an ability to administer higher total doses and improved efficacy while potentially incurring only minimal worsening of certain adverse events (AEs). Building on the Q1W dosing, other dosing schedules such as D1,8-Q3W will also be studied. The elimination of LV administration on D15 in a 21-day cycle in D1,8-Q3W enables hematologic recovery, and may prevent dose delays and dose elimination. It also enables more effective use of growth factors, allows subjects in a palliative care setting to not have a drug infusion each week, and provides a schedule (3 week cycles) that can be combined with most breast cancer and solid tumor regimens (e.g. pembrolizumab). To establish the maximum tolerated dose (MTD), a starting dose of 1.25 mg/kg will be administered on each of Day 1 and Day 8 of each 21-day cycle (for D1,8-Q3W) or a starting dose of 0.833 mg/kg will be administered on Day 1 of each 7-day cycle (for Q1W), to achieve an equivalent total dose of 2.5 mg/kg/cycle and similar exposure (AUC) as the previous Q3W dosing at 2.5 mg/kg.

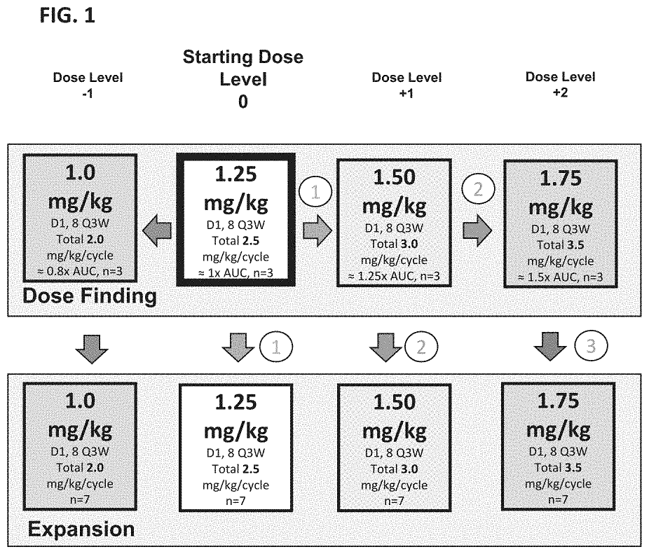

Specifically, human patients will be administered LV by intravenous (IV) infusion at a starting dose of 1.25 mg/kg on each of Day 1 and Day 8 of each 21-day cycle (D1,8-Q3W) (Starting Dose Level 0) (FIG. 1). Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. To monitor for treatment-related AEs, Blood tests or MRI scans will be conducted every week following the first administration of LV to assess and/or grade peripheral neuropathy (PN) as well as neutropenia. As shown in FIG. 1, if toxicity and treatment-related adverse events (AEs) are undesirable at Dose Level 0, the D1,8-Q3W dose will be decreased to 1.0 mg (Dose level −1). On the contrary, if the starting dose level 0 is determined to be below MTD, the next dose level (+1) will be opened for new enrollment and the dose level 0 will be expanded for more human patient enrollment. Similarly, if dose level (+1) was found to be below MTD, the next dose level (+2) was opened for new enrollment and the dose level +1 was expanded.

### Example 2: Dose and Approximate Relative AUC Equivalence

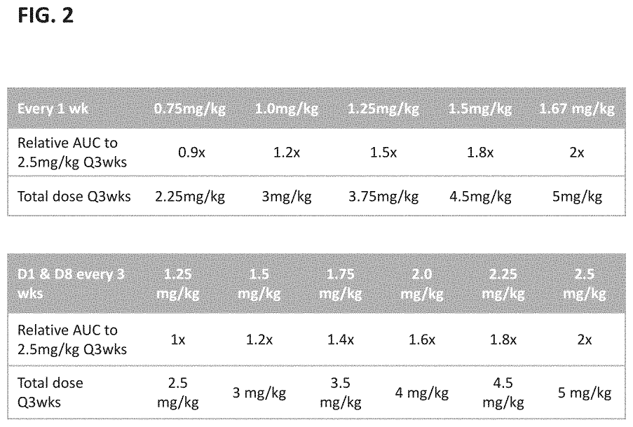

To compare total dose and relative exposure under weekly dosing schedule (Q1W) or D1,8-Q3W dosing schedule at different dose levels, the total dose administered in a 21-day cycle was calculated (Total dose Q3wks) and the simulated AUC was compared relative to that of Q3W dosing at 2.5 mg/kg. FIG. 2 displayed the total dose and approximate relative AUC equivalence in a weekly dosing schedule (Q1W) or D1,8-Q3W dosing schedule of LV.

### Example 3: Pharmacokinetics Modeling of ADC Species with Different DARs Under Tri-Weekly Dosing Scheme (Q3W) Vs. Weekly Dosing Scheme (Q1W)

In order to investigate pharmacokinetics of ADC species with different DARs under the previous tri-weekly dosing scheme (Q3W) versus a weekly dosing scheme (Q1W), LV was administered to subjects accordingly, and the pharmacokinetics of ADC species with different DARs were simulated.

Breast cancer patients were treated with ladiratuzumab vedotin (LV).

LV was administered by 30 minute intravenous (IV) infusion under a Q3W interval.

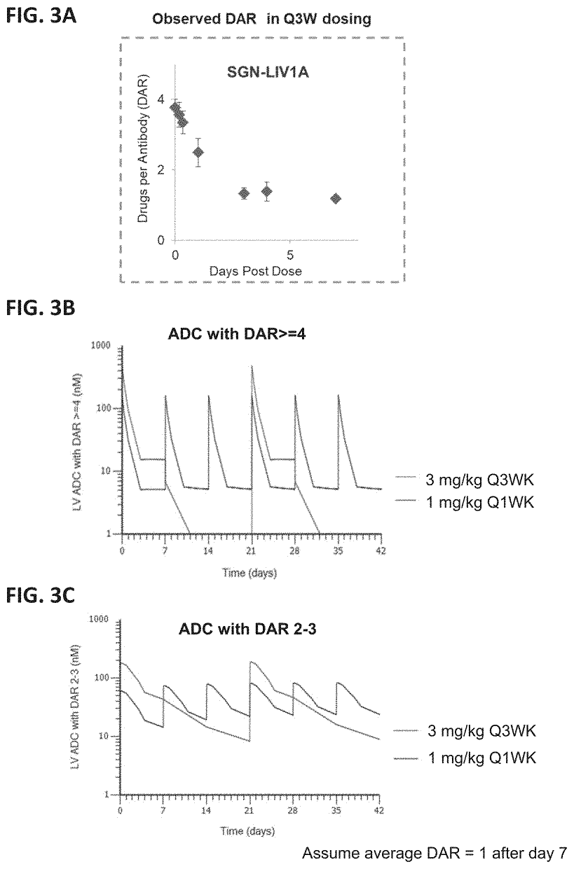

Following administration of LV, subject blood samples were collected at pre-dose, end of infusion, 2 hrs, 4 hrs, 8 hrs, 1 day, 3 days, 7 days, and 14 days after administration. The concentrations of the antibody-drug conjugate (ADC) was measured, and the drug to antibody ratio (DAR) of each species (DAR8, DAR7, DAR6, DAR5, DAR4, DAR3, DAR2, DAR1 and DAR0) of the ADCs collected in blood samples was also analyzed. As shown in FIG. 3A, the DAR of ADCs in Q3W dosing dropped significantly from about 4 (on Day 0) to about 1 (on Day 7). Using computer-assisted modeling, where decrease in average DAR was established previously, i.e. from 4 (on Day 0) to 1 (on Day 7) under Q3W, the pharmacokinetics were simulated for (i) ADCs with DARs ≥4, or (ii) ADCs with DAR of between 2 and 3 (FIGS. 3B, C respectively) when LV was administered at a dose of 1 mg/kg on Day 1 of each 7-day cycle (Q1W) or at a dose of 3 mg/kg on Day 1 of each 21-day cycle (Q3W). Briefly, based on the concentration of each ADC species from a single dose, a nonparametric superposition approach was used to predict steady state concentrations of each ADC species after multiple doses of LV, where predictions were based on an accumulation ratio from the terminal slope. As shown in FIG. 3B, concentrations for highly conjugated species dropped to zero for Q3W dosing due to fast clearance. In contrast, higher C_{trough} values were maintained for all ADC species with weekly dosing (Q1W), with significantly smaller peak to trough fluctuations (FIG. 3B, 3C).

### Example 4: Pharmacokinetics Modeling of ADC Species with Different DARs Under a Weekly Dosing (Q1W) or a Modified Dosing Scheme (D1,8-Q3W)

In order to investigate pharmacokinetics of ADC species with different DARs under a weekly dosing (Q1W) or a modified dosing scheme (D1,8-Q3W), LV was administered to subjects, and the pharmacokinetics of ADC species and MMAE species were simulated.

Breast cancer patients were treated with ladiratuzumab vedotin (LV).

LV was administered by intravenous (IV) infusion at a dose of 3 mg/kg under a Q3W interval. LV was administered as 30 minute IV infusion.

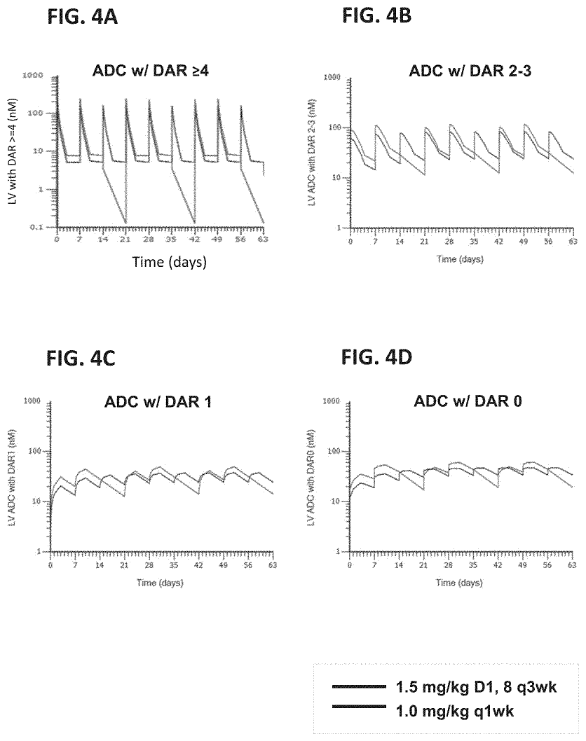

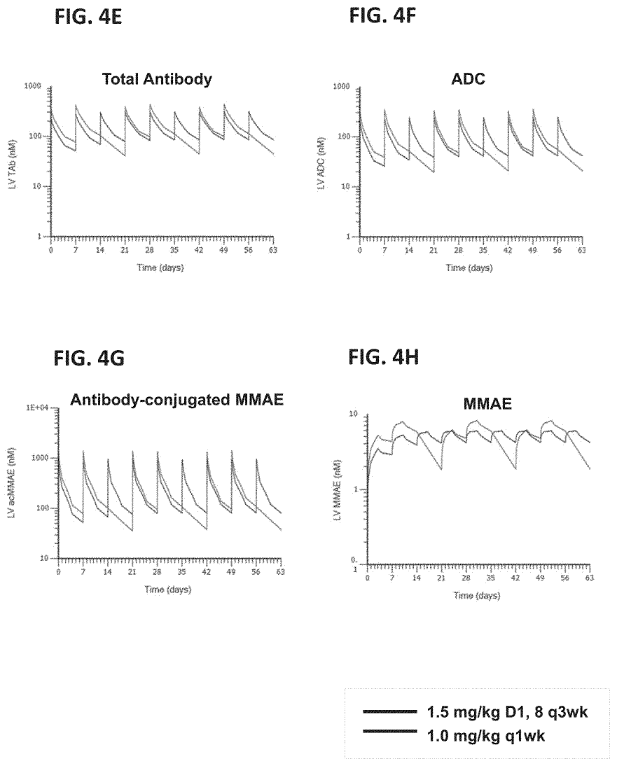

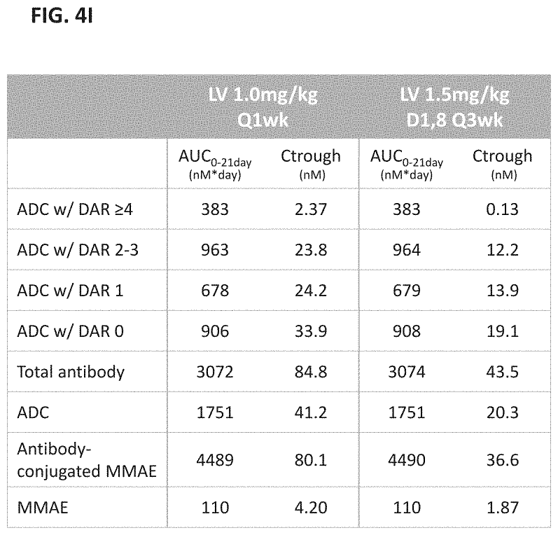

The concentrations of the antibody-drug conjugate (ADC), free MMAE and antibody-conjugated MMAE were simulated based on measured ADC, MMAE concentration, and average DAR under a Q3W interval, respectively. The drug to antibody ratio (DAR) of each species (DAR8, DART, DARE, DAR5, DAR4, DAR3, DAR2, DAR1 and DAR0) of the ADCs collected in blood samples from Q3W were analyzed. Using computer-assisted modeling, where decrease in average DAR was established from the above measurements under Q3W dosing, the pharmacokinetics were simulated for ADCs with DARs ≥4 (FIG. 4A), ADCs with DAR of between 2 and 3 (FIG. 4B); ADCs with DAR=1 (FIG. 4C); and ADCs with DAR=0 (FIG. 4D) when LV was administered according to Q1W or D1,8-Q3W dosing schedule. Briefly, based on the concentration of each ADC species from a single dose, a nonparametric superposition approach was used to predict steady state concentrations of each ADC species after multiple doses of LV, where predictions were based on an accumulation ratio from the terminal slope. The pharmacokinetics were also simulated for total antibody (FIG. 4E); total ADCs (FIG. 4F); antibody-conjugated MMAEs (FIG. 4G) and free MMAEs (FIG. 4H) when LV was administered according to Q1W or D1,8-Q3W dosing schedule. The corresponding area under curve after 21 days (AUC₀₋₂₁day) and C_{trough} values were calculated from the simulations for the mentioned species under the described dosing conditions (FIG. 41). As shown in FIG. 4A, concentrations for highly conjugated species dropped more rapidly under D1,8-Q3W dosing due to fast clearance and drug holiday at D15. The data also showed that while the total dosage as well as the AUC were similar for the two dosing schemes, higher C_{trough} values were achieved for total antibody, total ADC, MMAE and all species of ADC under weekly dosing (Q1W) (FIG. 41), with smaller peak to trough fluctuations (FIG. 4A-4H)

### Example 5: Pharmacokinetics of ADC Species of BV and LV Under a Weekly Dosing Schedule (Q1WK)

In order to further investigate pharmacokinetics of ADC species with different DARs under Q1WK dosing, ladiratuzumab vedotin (LV) and brentuximab vedotin (BV) were administered to subjects, and the pharmacokinetics of ADC species were simulated.

Breast cancer patients were treated with BV or LV.

BV and LV were respectively administered by 30 minute intravenous (IV) infusion under a Q3W interval.

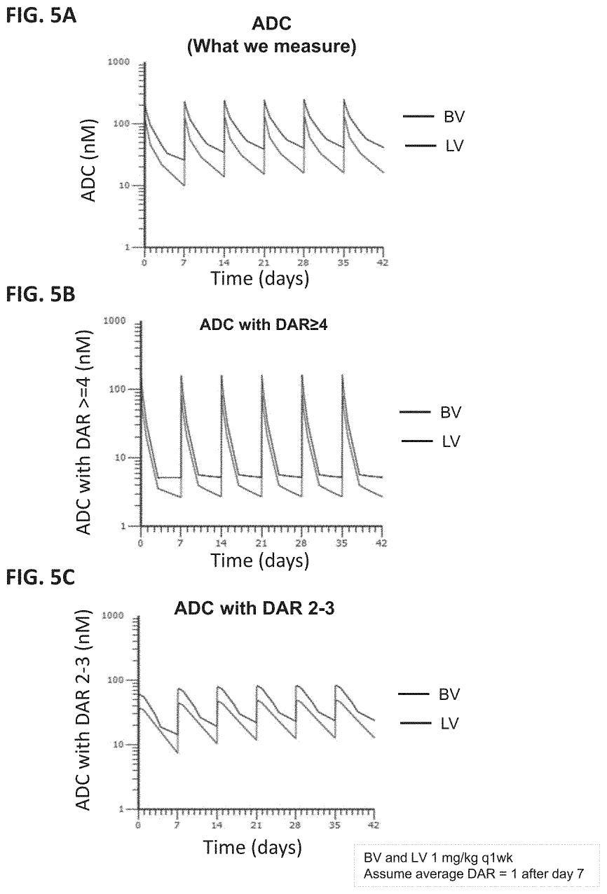

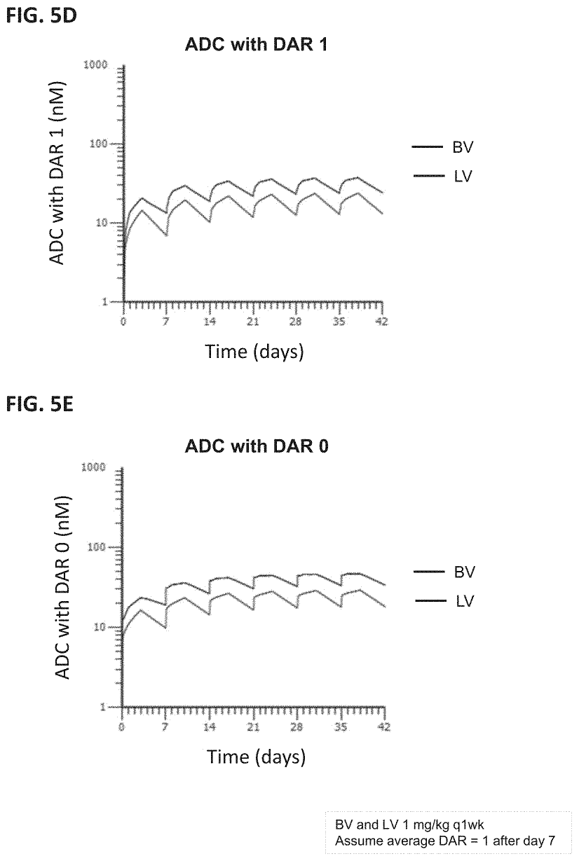

The concentration of the antibody-drug conjugate (ADC) of BV and LV was simulated based on measured ADC concentration under Q3W dosing (FIG. 5A). BV and LV contain MMAE conjugated to the antibody using the same linker. The drug to antibody ratio (DAR) of each species (DAR8, DART, DARE, DAR5, DAR4, DAR3, DAR2, DAR1 and DAR0) of the ADCs measured in blood samples from LV treated patients were applied to BV as well. Using computer-assisted modeling, where decrease in average DAR was established from the above measurements under Q3W dosing, the pharmacokinetics were simulated for ADCs with DARs ≥4 (FIG. 5B), ADCs with DAR of between 2 and 3 (FIG. 5C); ADCs with DAR=1 (FIG. 5D); and ADCs with DAR=0 (FIG. 5E). Briefly, based on the concentration of each ADC species from a single dose, a nonparametric superposition approach was used to predict steady state concentrations of each ADC species after multiple doses of ADC, where predictions were based on an accumulation ratio from the terminal slope. While the PK of total ADC appeared different between BV and LV (FIG. 5A), the antibody-conjugate MMAE is more predictive of safety and efficacy. Specifically, ADC species with higher DAR would deliver more MMAE to cells. As shown in FIG. 5B, ADC species with high DAR (DAR ≥4) exhibited faster clearance than ADC species with lower DARs (FIGS. 5C, 5D, 5E). Nevertheless, the C_{trough} for ADC species with DAR ≥4 was still well above 1 nM for BV and LV, indicating that the Q1W dosing schedule could improve efficacy, and the improvement was similarly observed for BV and LV. In addition, the improvement in C_{trough} values and the reduction of peak to trough fluctuations for ADC species were similar for BV and LV, indicating applicability of the Q1W dosing regimen across multiple kinds of ADCs.

### Example 6: Correlation of LV Pharmacokinetics and Treatment Efficacy

To investigate the correlation of pharmacokinetics of ADCs with treatment efficacy, subjects were treated with LV and analyzed for probability of response in relation to observed ADC and MMAE pharmacokinetics.

Breast cancer patients were treated with ladiratuzumab vedotin (LV).

LV was administered by 30 minute intravenous (IV) infusion at doses of 0.5-2.8 mg/kg under a Q3W interval.

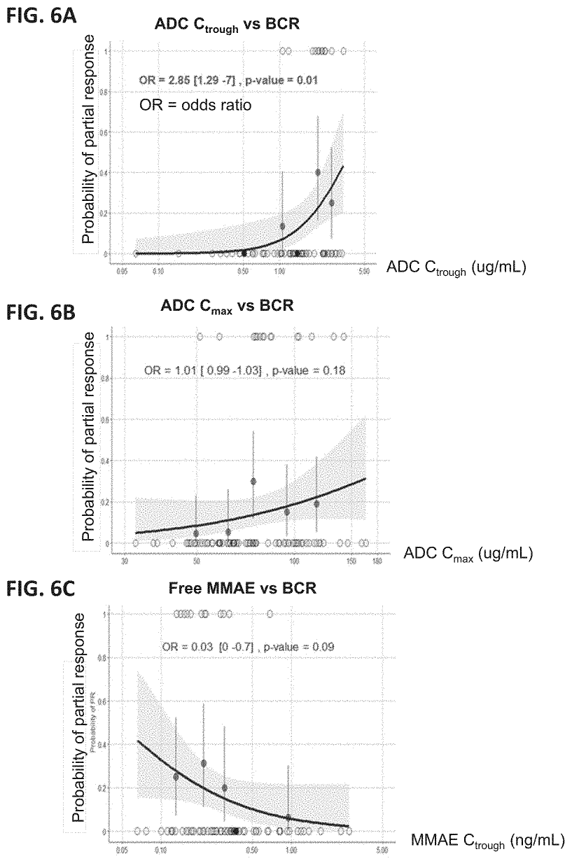

Following administration of LV, subject blood samples were collected at pre-dose, end of drug administration, 2 hrs, 4 hrs, 8 hrs, 1 day, 3 days, 7 days, and 14 days after administration. The concentrations of the antibody-drug conjugate (ADC) and free MMAE were measured. Using measurements of MMAE or ADC concentrations, patients were binned to <=20%, >20-<=40%, >40-<=60%, >60-<=80% and >80% percentiles according to the calculated AUC_{0-21 day}, Cₘₐₓ or C_{trough} of ADC and MMAE. Tumor assessment according to RECIST v1.1 was performed every 6 weeks (±3 days) for the first 12 months and then every 12 weeks (±7 days) thereafter. Objective responses were confirmed with repeat scans 4-6 weeks after the first documentation of response. The probability of best confirmed response (BCR) in the binned patients were plotted against ADC C_{trough}, ADC Cₘₐₓ or MMAE C_{trough} (FIG. 6A, 6B, 6C respectively). As shown from FIG. 6A-C, logistic regressions of best confirmed response (BCR) by ADC C_{trough} was most significant. Treatment efficacy also correlates with AUC (not shown), but not with ADC Cₘₐₓ (FIG. 6B). In addition, there is no endpoint efficacy correlation with free MMAE (FIG. 6C). Taken together, out of the various pharmacokinetic parameters, the C_{trough} values are highly predictive of probability of response to administration of ADCs.

### Example 7: Probability of Adverse Events Under Tri-Weekly Dosing (Q3W) Vs. Weekly Dosing (Q1W) of BV

To investigate the correlation of dosing regimens with probability of AEs, subjects were treated with BV and analyzed for occurrence of AEs at different standardized dose levels when ADCs are administered under tri-weekly dosing (Q3W) or weekly dosing (Q1W).

Patients were treated with brentuximab vedotin (BV).

BV was administered by intravenous (IV) infusion at either: (a) one dose on Day 1 of each 7-day cycle (Q1W); or (b) one dose on Day 1 each 21-day cycle (Q3W), wherein the patients receive dose levels standardized at 0.4, 0.45, 0.6, 0.75, 0.9, 1.05, or 1.2 mg/kg/wk respectively for either dosing schedule. BV was administered as 30 minute IV infusion at the indicated interval.

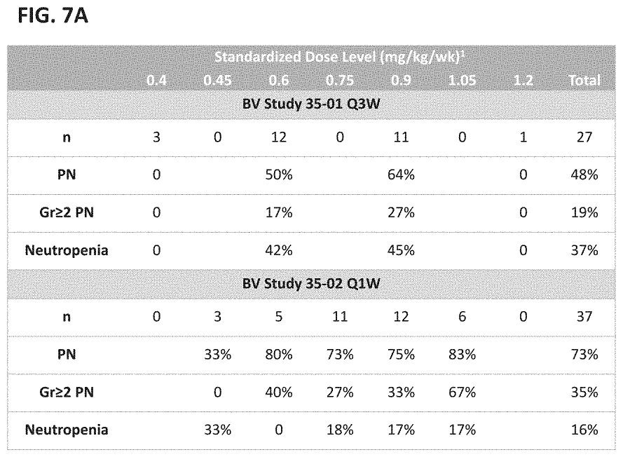

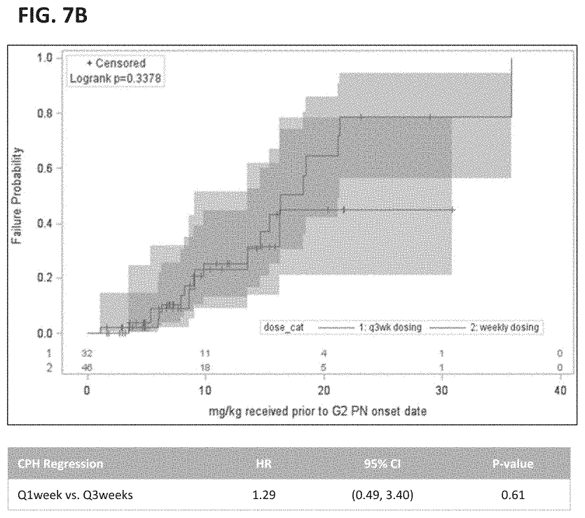

Subjects were binned according to the described standardized dose levels for Q1W or Q3W dosing schedule. To monitor for treatment-related AEs, Blood tests or MRI scans were conducted every week following the first administration of BV to assess and/or grade peripheral neuropathy (PN) as well as neutropenia. FIG. 7A showed the percentage of patients exhibiting PN, PN at or above Grade 2 (Gr≥2 PN), or neutropenia under Q3W or Q1W dosing schedule at the indicated standardized dose levels. Comparing the standardized dose levels of 0.6 and 0.9 (FIG. 7A) as well as the plot of AE probability against total dose administered (FIG. 7B), a multivariate cumulative exposure model indicated there was a trend for higher risk of overall PN under Q1W dosing (HR=0.58 (0.29, 1.15), p=0.12). The trend for higher risk of Gr≥2 PN under Q1W was lower comparatively (HR=0.76 (0.27, 2.20), p=0.61). On the other hand, the risk for neutropenia was significantly lower (69%) for Q1W dosing as compared to Q3W dosing (OR=0.31 (0.09, 1.04), p=0.06). The results show that Q1W dosing can lead to overall lower incidence of severe adverse effects, such as lower risk of neutropenia while maintaining the same standardized dose level.

### Example 8: Fractionated Dosing Decreases Cₘₐₓ and Peak-to-Trough Fluctuation, while Maintaining Similar Total Exposure

To investigate if fractionated dosing could increase treatment efficacy, various fractionated dosing schemes of LV were tested to measure for changes in Cₘₐₓ and peak-to-trough fluctuations at similar total dose intensities and exposure.

Breast cancer patients were treated with ladiratuzumab vedotin (LV).

LV was administered by intravenous (IV) infusion at either: (1) a dose of 2.5 mg/kg on Day 1 of each 21-day cycle (D1Q3wk); (2) a dose of 0.75 mg/kg on Day 1 of each 7-day cycle (QW); (3) a dose of 1 mg/kg on each of Day 1, Day 8 and Day 15 of each 28-day cycle (D1,8,15-Q4wk); or (4) a dose of 1.25 mg/kg on each of Day 1 and Day 8 of each 21-day cycle (D1,8-Q3wk) to achieve comparable dose intensities (at 0.83, 0.75, 0.75 and 0.83 mg/kg/wk respectively). LV was administered as 30 minute IV infusion at the indicated interval.

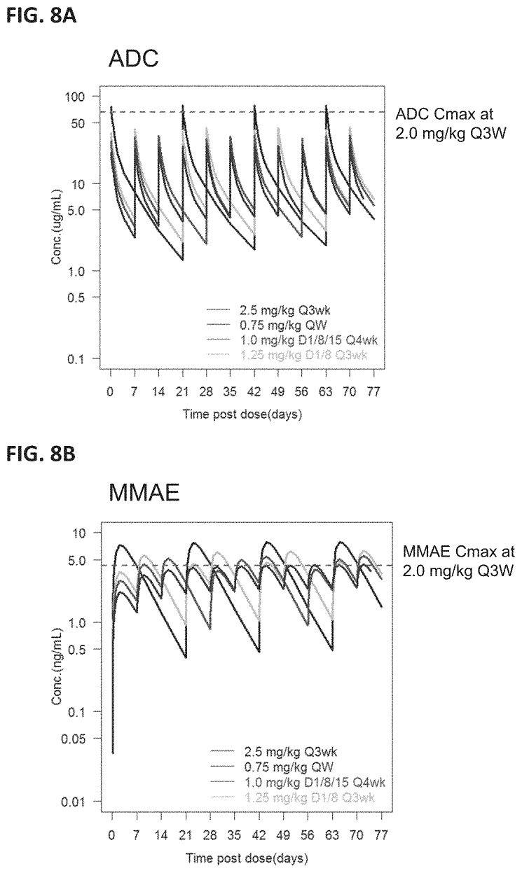

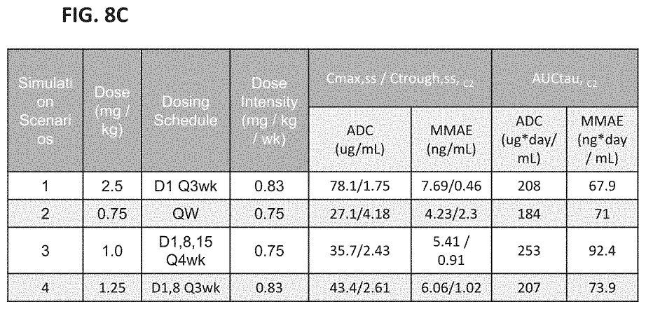

Using compartmental population PK modeling, the pharmacokinetics were simulated for mean concentration of total ADC and MMAE for 11 week after the first dose for all the four dosing schedules described above (FIGS. 8A, 8B respectively). Briefly, based on the measured concentration of total ADC and free MMAE under Q3W dosing interval, a compartmental population PK model was established. Total ADC PK was described by a linear, three-compartment model with first-order elimination. MMAE PK was described by a semi-mechanistic, linear 1-compartment model with first-order elimination. MMAE was assumed to form from both ADC proteolytic degradation and deconjugation processes. The average DAR was assumed to decrease exponentially after each dose. Established population PK model was used to predict total ADC and MMAE concentrations after multiple doses of LV. The Cₘₐₓ, C_{trough}, as well as AUCₜₐᵤ for both ADC and MMAE were projected. As demonstrated in FIG. 8C, fractionated dosing (such as QW, D1,8,15-Q4wk or D1,8-Q3wk) significant decreased Cₘₐₓ of ADC and reduced peak-to-trough fluctuations for both ADC and MMAE as compared to a concentrated dosing (D1Q3wk), while maintaining similar total exposure (as determined by AUCₜₐᵤ).

### Example 9: Fractionated Dosing Increases Total Exposure while Maintains Similar MMAE Cₘₐₓ

To investigate if fractionated dosing could increase treatment efficacy, various fractionated dosing schemes of LV were tested to measure for changes in total exposure in relation to pharmacokinetics of MMAE.

Breast cancer patients were treated with ladiratuzumab vedotin (LV).

LV was administered by intravenous (IV) infusion at either: (a) a dose of 2.5 mg/kg on Day 1 of each 21-day cycle (Q3wk), or (b) a dose on each of Day 1 and Day 8 of each 21-day cycle (D1,8-Q3wk) where each dose was either 1.0, 1.25, 1.5, or 1.75 mg/kg. LV was administered as 30 minute IV infusion at the indicated interval.

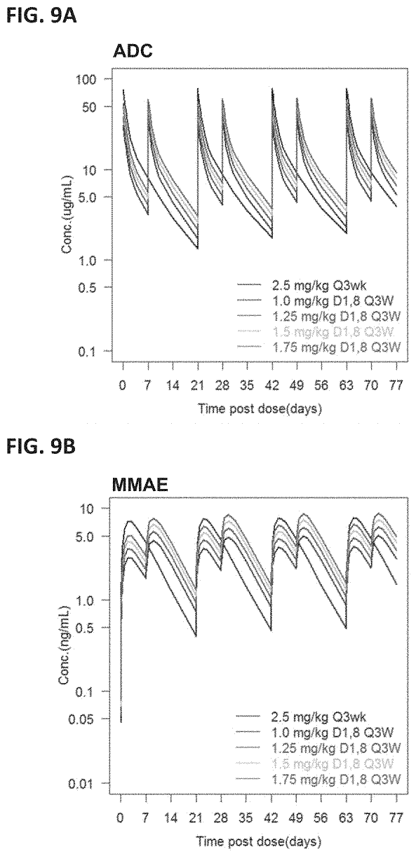

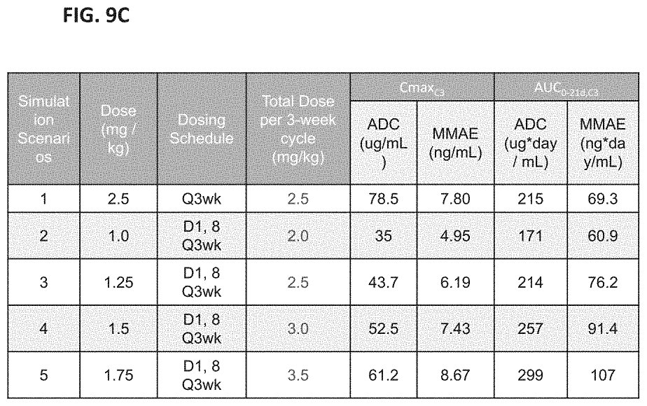

Using compartmental population PK modeling, the pharmacokinetics of ADC and MMAE were simulated under multiple doses for all five dosing schedules described above (FIG. 9A, 9B respectively). Briefly, based on the measured concentration of ADC and MMAE under Q3W dosing, a compartmental population was established. This model (as described in Example 8) was used to predict steady state concentrations of ADC and MMAE after multiple doses of LV. The Cₘₐₓ, as well as AUC₀₋₂₁d for both ADC and MMAE were projected. As demonstrated in FIG. 9C, fractionated dosing (e.g. D1,8-Q3wk at 1.0, 1.25, 1.5 mg/kg) decreased Cₘₐₓ of both ADC and MMAE as compared to a concentrated dosing (Q3wk), while maintaining similar total dose per 3-week cycle (˜2.0 to 3.0 mg/kg). In particular, at a dosing schedule of D1,8-Q3wk at 1.5 mg/kg, the total exposure of ADC and MMAE was significantly higher compared to Q3wk (as observed by AUC₀₋₂₁d values) while similar MMAE Cₘₐₓ was maintained (FIG. 9C).

### Example 10: Fractionated Dosing of Ladiratuzumab Vedotin Maintained Lower Cₘₐₓ and Higher C_{trough} Compared to Q3W

To investigate if fractionated dosing could increase treatment efficacy or reduce the number and severity of adverse events, various fractionated dosing schemes of ladiratuzumab vedotin (LV) were tested to measure for changes in Cₘₐₓ, C_{trough}, and AUC at similar total dose intensities and exposure.

Breast cancer patients were treated with (LV).

LV was administered by intravenous (IV) infusion at either: (1) a dose of 2.5 mg/kg on Day 1 of each 21-day cycle (D1Q3wk); (2) a dose of 1 mg/kg on each of Day 1 each 7-day cycle (Q1W); or (3) a dose of 1.25 mg/kg on each of Day 1 each 7-day cycle (Q1W) to achieve comparable dose intensities (at 0.83, 1.0, and 1.25 mg/kg/wk respectively). LV was administered as 30-minute IV infusion at the indicated interval.

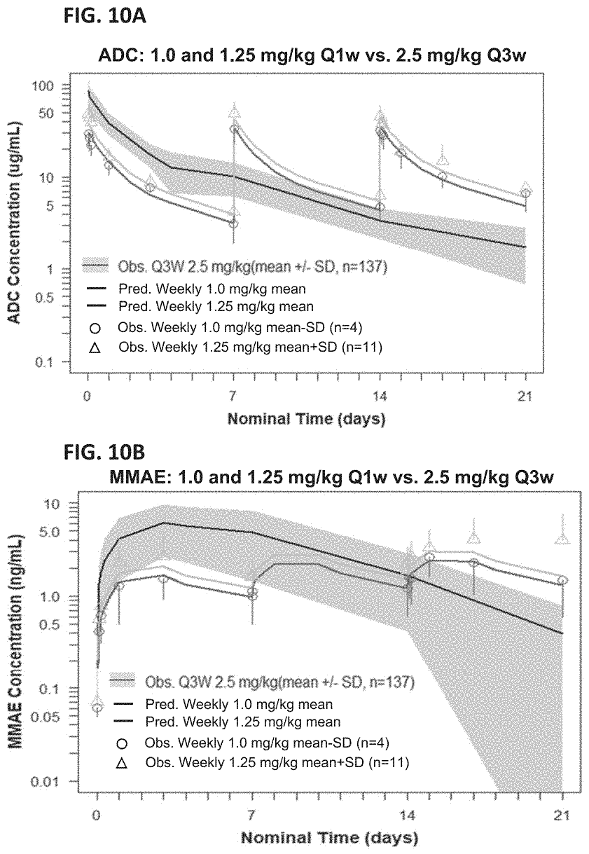

Using compartmental population PK modeling, the pharmacokinetics were simulated for mean concentration of total ADC and MMAE for 21 days after the first dose for all three dosing schedules described above (FIGS. 10A, 10B respectively). Briefly, based on the measured concentration of total ADC and free MMAE under Q1W dosing interval, a compartmental population PK model was established. Total ADC PK was described by a linear, three-compartment model with first-order elimination. MMAE PK was described by a non-compartmental superposition model. MMAE was assumed to form from both ADC proteolytic degradation and deconjugation processes. The average DAR was assumed to decrease exponentially after each dose. Established population PK model was used to predict total ADC and MMAE concentrations after multiple doses of LV. The Cₘₐₓ, C_{trough}, as well as AUCₜₐᵤ for both ADC and MMAE were projected. As demonstrated in FIG. 10A and FIG. 10B, fractionated dosing (Q1W) significantly decreased Cₘₐₓ of ADC and resulted in higher C_{trough} for both ADC and MMAE as compared to a concentrated dosing (D1Q3wk), while maintaining similar total exposure (as determined by AUC₀₋₂₁). These models were experimentally confirmed as shown in FIG. 10A and FIG. 10B and the following tables, demonstrating that the model accurately predicted the ADC concentration and MMAE concentration.

| | Cₘₐₓ | AUC₀₋₂₁ | C_{trough} |
|---|---|---|---|
| | (μg/mL) | (μg*day/mL) | (μg/mL) |
| 1.0 mg/kg weekly | 31.4 (25) | 215.4* | 6.32 (41) |
| (obs.) (n = 4) | | | |
| 1.0 mg/kg weekly | 35.0 | 207.1 | 4.88 |
| (Pred.) | | | |
| 1.25 mg/kg weekly | 43.3 (31) | 266.9* | 7.64 |
| (obs.) (n = 5) | | | (n = 1) |
| 1.25 mg/kg weekly | 43.8 | 258.9 | 6.1 |
| (Pred.) | | | |
| 2.5 mg/kg q3wk | 85 (27) | 210 (27) | 1.62 (58) |
| (obs.) | (n = 131) | (n = 97) | (n = 96) |
| Geo. mean (CV %) | | | |

*Estimated from observed AUC_{0-7 d} of Cycle 1 Dose #1 and Dose #3.

| | Cₘₐₓ | | AUC₀₋₂₁ | C_{trough} | |
|---|---|---|---|---|---|
| | (μg/mL) | | (μg*day/mL) | (μg/mL) | |
| 1.0 mg/kg weekly | 2.1 | (61) | 29.7* | 1.26 | (75) |
| (obs.) (n = 4) | | | | | |
| 1.0 mg/kg weekly | 2.40 | | 34.7 | 1.30 | |
| (Pred.) | | | | | |
| 1.25 mg/kg weekly | 3.74 | (68) | 53.6* | 2.95 | (160) |
| (obs.) (n = 5) | | | | (n = 2) | |
| 1.25 mg/kg weekly | 2.98 | | 43.4 | 1.65 | |
| (Pred.) | | | | | |
| 2.5 mg/kg q3wk | 5.55 | (56) | 50.3 (61) | 0.34 | (80) |

(obs.)
Geomertic mean
(CV %)

*Estimated from observed AUC_{0-7 d} of Cycle 1 Dose #1 and Dose #3.

### Example 11: Anti-Tumor Activity of Ladiratuzumab Vedotin at Various Dosing Schedules in Patients with Estrogen Receptor Positive (ER+) Breast Cancer

Human patients with ER+ breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 12: Anti-Tumor Activity of Ladiratuzumab Vedotin at Various Dosing Schedules in Patients with Progesterone Receptor Positive/Human Epidermal Growth Factor Receptor 2 Negative (PR+/HER2−) Breast Cancer

Human patients with PR+/HER2− breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 13: Anti-Tumor Activity of Ladiratuzumab Vedotin at Various Dosing Schedules in Patients with ER+/PR+/HER2− Breast Cancer

Human patients with ER+/PR+/HER2− breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 14: Anti-Tumor Activity of Ladiratuzumab Vedotin at Various Dosing Schedules in Patients with Tripe Negative Breast Cancer

Human patients with triple negative breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 15: Anti-Tumor Activity of Ladiratuzumab Vedotin at Various Dosing Schedules in Patients with Hormone Receptor Positive (HR+) Breast Cancer

Human patients with HR+ breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 16: Anti-Tumor Activity of Ladiratuzumab Vedotin at Various Dosing Schedules in Patients with HER2 Positive Breast Cancer

Human patients with HER2 positive breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 17: Anti-Tumor Activity of Ladiratuzumab Vedotin at Various Dosing Schedules in Patients with HR+/HER2 Negative Breast Cancer

Human patients with HR+/HER2 negative breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 18: Anti-Tumor Activity of Ladiratuzumab Vedotin at Various Dosing Schedules in Patients with Estrogen Receptor Positive/Human Epidermal Growth Factor Receptor 2 Negative (ER+/HER2−) Breast Cancer

Human patients with ER+/HER2− breast cancer will be treated with ladiratuzumab vedotin (LV). Patients will have cancers that express LIV1.

LV will be administered by intravenous (IV) infusion at either: (a) a dose of 1 mg/kg on Day 1 of each 7-day cycle (Q1W); (b) a dose of 1.5 mg/kg on each of Day 1 and Day 8 of each 21-day cycle (D1,8-Q3W); or (c) a dose of 3 mg/kg on Day 1 of each 21-day cycle (Q3W). An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered as 30 minute IV infusion at the indicated interval.

### Example 19: A Phase 1, Open-Label, Dose-Escalation Study to Evaluate the Safety and Tolerability of Ladiratuzumab Vedotin in Patients with Metastatic Breast Cancer

This is a phase 1, open label, dose escalation study to evaluate the safety and tolerability of ladiratuzumab vedotin (LV) in patients with metastatic breast cancer.

Eligible patients are females at least 18 years of age who have incurable, unresectable, locally advanced or metastatic breast cancer (LA/mBC). Patients must have pathologically and radiologically confirmed hormone receptor-positive/human epidermal growth factor 2 negative (HR+/HER2−) or metastatic triple-negative breast cancer (mTNBC) with at least 1 measurable lesion per RECIST v1.1. Subjects with HR+/HER2− disease must have received no more than 1 prior line of cytotoxic chemotherapy in the locally advanced (LA)/mBC setting, either as single agent or combination therapy. Subjects with TNBC must have received 1 prior line of cytotoxic chemotherapy in the LA/mBC setting. Progression within 6 months of completion of neoadjuvant therapy is considered an LA/mBC regimen. Enrollment is open to all patients, irrespective of their LIV-1 expression level. Available and adequate archival baseline tumor sample is required. A fresh biopsy sample may be submitted in place of an archival sample if medically feasible.

Up to 82 subjects will be enrolled (42 HR+/HER2− and 42 mTNBC) into dose escalation and dose-expansion cohorts. Patients will receive LV on Day 1, Day 8, and Day 15 in every 3-week cycle. The study will have dose escalation and expansions cohorts. Patients with HR+/HER2− and TN disease will be enrolled into separate dose-expansion cohorts for each dose level.

Dose escalation will be conducted using the modified toxicity probability interval (mTPI) method according to Ji Y. et al. Clin Trials 7(6):653-63 (2010). Only 1 dose-escalation cohort will be open at a time. Dose-expansion cohorts may be opened at any dose level that has cleared dose-limiting toxicity (DLT) evaluation. Initially, up to approximately 10 patients will be enrolled at each dose level. DLT evaluation will only include data from the dose-escalation cohorts, but the totality of data from all patients at each dose level will be used to determine the recommended weekly LV dose.

LV will be administered at a dose of 0.75 mg/kg to 1.75 mg/kg by intravenous infusion over the course of 30 minutes on Day 1, Day 8, and Day 15 in every 3-week cycle.

Patients may continue on treatment until disease progression or unacceptable toxicity.

Antitumor activity will be assessed by radiographic tumor imaging at protocol-specified time points. Response will be categorized by RECIST Version 1.1.

Safety assessments will consist of the surveillance of adverse events (AEs), laboratory test measures, physical examination findings, electrocardiograms, vital signs, and concomitant medication records.

### Objectives

Primary Objective
To evaluate the safety and tolerability of LV in patients with incurable, unresectable locally advanced or metastatic breast cancer (LA/MBC).
To identify the maximum tolerated dose (MTD) of LV, if one exists among doses and schedules tested.

Secondary Objectives
To assess the pharmacokinetics of LV.
To assess the immunogenicity of LV.
To assess the antitumor activity of LV.

Additional Objectives
To assess exploratory biomarkers of LV-mediated pharmacodynamic effects and to evaluate LIV-1 expression in tumor tissue.
To evaluate the LIV-1 expression-response relationship following treatment with LV.

### Endpoints

Safety Endpoints
Type, incidence, severity, seriousness, and relatedness of adverse events (AEs)
Type, incidence, and severity of laboratory abnormalities
Incidence of DLT

Efficacy Endpoints
ORR, confirmed and unconfirmed
Duration of response (DOR)
Progression-free survival (PFS)
OS
PFS ratio relative to prior therapy

Patients will be evaluated for response after every 2 cycles of treatment in the first 10 cycles and after every fourth cycle thereafter; patients who achieve stable disease (SD) or better according to Response Evaluation Criteria for Solid Tumors (RECIST) Version 1.1 will be eligible to continue receiving study treatment until disease progression or unacceptable toxicity. In the event that a patient tolerates at least 4 cycles of LV and achieves a partial response (PR) or SD, additional treatment cycles at the highest dose level considered to be tolerable may be administered upon approval by the medical monitor.

### Dosing

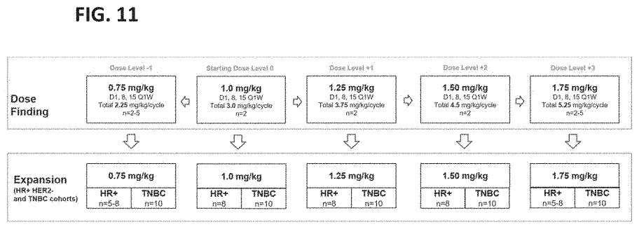

The dose escalation and dose expansion scheme is described in FIG. 11. Dose escalation will begin at dose level 0. The first patients enrolled at each dose level will be evaluated for DLTs. If a dose clears DLT evaluation, subsequent patients may be enrolled at the next dose level or in the dose-expansion cohorts as shown. Up to approximately 10 patients will be enrolled at each dose level.

The starting dose of LV will be dose level 0 at 1.0 mg/kg on Day 1, Day 8, and Day 15. Planned dose levels are described in the following table:

| | | Maximum LV dose |
|---|---|---|
| | Weekly LV Dose | in a 3-week cycle |
| Dose Level | (mg/kg) | (mg/kg/cycle) |
| −1 | 0.75 | 2.25 |
| 0 | 1.00 | 3.00 |
| 1 | 1.25 | 3.75 |
| 2 | 1.50 | 4.50 |
| 3 | 1.75 | 5.25 |

Only 1 dose level will be open at a time in dose escalation to allow for DLT evaluation. DLT evaluation will only include data from dose-escalation cohorts.

Dose escalation will use the modified toxicity probability interval (mTPI) method. Ji Y. et al. Clin Trials 7(6):653-63 (2010). The mTPI method is a model-based dose-escalation design that uses a Bayesian statistical framework and a beta-binomial hierarchic model. Using a target DLT rate of 25% with a 5% margin, the dosing-decision rules are:
1. Escalate if current DLT rate is most likely <20%
2. Continue if current DLT rate is most likely between 20% and 30%
3. De-escalate if current DLT rate is most likely >30%

Each dose cohort will start with 2 patients. Escalation to the next dose level will occur if 0 of the 2 patients encounter a DLT, while de-escalation will occur if both patients encounter a DLT. If 1 of the 2 patients encounter a DLT, an additional 4 patients will be accrued (total of 6 DLT-evaluable patients). Escalation to the next dose level will occur if ≤1 of the 6 patients encounter a DLT. The MTD will be estimated based on data from all patients across all evaluated dose levels.

| Number of | | | | | |
|---|---|---|---|---|---|
| Patients | Number of DLT-Evaluable Patients Treated at Current Dose | | | | |
| with DLTs | 2 | 3 | 4 | 5 | 6 |
| 0 | Escalate | Escalate | Escalate | Escalate | Escalate |
| 1 | Stay | Stay | Stay | Stay | Escalate |
| 2 | De-escalate^{a} | De-escalate | De-escalate | Stay | Stay |
| 3 | | Unacceptable | Unacceptable | Unacceptable | De-escalate |
| | | Dose, De- | Dose, De- | Dose, De- | |
| | | escalate | escalate | escalate | |
| 4 | | | Unacceptable | Unacceptable | Unacceptable |
| | | | Dose, De- | Dose, De- | Dose, De- |
| | | | escalate | escalate | escalate |
| 5 | | | | Unacceptable | Unacceptable |
| | | | | Dose, De- | Dose, De- |
| | | | | escalate | escalate |
| 6 | | | | | Unacceptable |
| | | | | | Dose, De- |
| | | | | | escalate |

^{a}Dose will be de-escalated if the first 2 DLT-evaluable patients in a dose level experience a DLT. Further evaluation of that dose level may be allowed.

### DLT Criteria

The DLT evaluation period is the first treatment cycle (3 weeks). A DLT will be defined as any AE ≥Grade 3, according to the NCI CTCAE v4.03, that is possibly, probably, or definitely related to treatment with LV, unless evidence exists that the AE has an etiology other than the investigational agent (e.g., disease progression, pre-existing medical condition, underlying disease, intercurrent illness, or concomitant medication), and/or the event has no plausible temporal relationship to administration of the investigational agent.

The following events will not be considered a DLT:
Any AE clearly related to disease progression or concomitant disease (e.g., diabetes)
Asymptomatic grade 3 non-hematologic laboratory abnormalities which resolve to grade 1 or baseline within 14 days
Grade 3 or 4 lymphopenia will not be considered a DLT
Grade 3 thrombocytopenia will not be considered a DLT unless it does not resolve to grade 2 within 14 days or is associated with clinically significant bleeding
Grade 3 or 4 neutropenia or anemia will not be considered a DLT unless one of the following applies:
Grade 4 neutropenia lasts >7 days or results in hospitalization for fever or infection
Grade 4 anemia that is not explained by the underlying disease
Grade 3 allergic reactions

### Concurrent Dose Expansion

Dose-expansion cohorts may be opened at any dose level that has cleared DLT evaluation. Up to approximately 10 patients with HR+/HER2-negative disease will be enrolled at each dose level across the dose escalation and dose-expansion cohorts. Examples of the number of patients enrolled in DLT evaluation and dose expansion are shown in the following table:

| Patients required for | Patients that may be | Total number |
|---|---|---|
| DLT evaluation at | enrolled in dose expansion | of patients at |
| each dose level | at each dose level | each dose level |
| 2 | 8 | 10 |
| 3 | 7 | 10 |
| 4 | 6 | 10 |
| 5 | 5 | 10 |
| 6 | 4 | 10 |

The first patients enrolled at each dose level will be evaluated for DLTs. If a dose clears DLT evaluation, subsequent patients may be enrolled at the next dose level or in the dose-expansion cohorts shown above. Up to approximately 10 patients will be enrolled at each dose level.

Patients with mTNBC will not be enrolled in the dose-escalation cohort. There will be a separate dose-expansion cohort at each dose level for patients with mTNBC that will enroll up to approximately 10 patients.

The totality of data from all patients in dose escalation and expansion will be used to determine the recommended weekly LV dose. After the weekly dose has been determined, additional patients (up to 30) may be enrolled at that dose or lower.

### Inclusion Criteria

1. Pathologically confirmed diagnosis of breast cancer with radiographic evidence of incurable, unresectable, locally advanced or metastatic disease.

2. Patients with BR+/HER2-negative disease who are chemotherapy-eligible and are not considered a candidate for further hormonal therapy.
i. Patients will be considered HR+ if biopsies show >1% expression of ER or PR per current American Society of Clinical Oncology/College of American Pathologists (ASCO/CAP) 2018 guidelines.
ii. Must have progressed on or relapsed after receiving endocrine or hormonally-directly therapy with CDK inhibitors.
iii. Must have received no more than 1 prior cytotoxic regimen in the incurable, unresectable LA/MBC setting

3. Available and adequate archival baseline tumor sample is required.

4. Measureable disease as defined in RECIST Version 1.1: at least 1 tumor lesion ≥10 mm in the longest diameter or a lymph node ≥15 mm in short axis measurement assessed by CT (computed tomography) scan.

5. Females ≥18 years of age.

6. An Eastern Cooperative Oncology Group (ECOG) performance status score of 0 or 1.

7. Patients must have completed treatment with chemotherapy, radiotherapy, hormonal therapy, or other treatment with an investigational agent ≥2 weeks prior to first dose of study drug, unless disease progression is documented, and have recovered from any clinically significant toxicity associated with the treatment.

8. Patients must have completed treatment with a biologic agent or immunotherapy ≥4 weeks prior to the first dose of study drug, unless disease progression is documented, and have recovered from any clinically significant toxicity associated with the treatment. An exception to this requirement is treatment with denosumab, which is permitted on study.

9. The following baseline laboratory data:
absolute neutrophil count (ANC)≥1500/μL,
platelet count ≥100,000/μL
hemoglobin (Hgb)≥8.0 g/dL
serum bilirubin ≤1.5× upper limit of normal (ULN)
serum creatinine ≤1.5×ULN
alanine aminotransferase (ALT) and aspartate aminotransferase (AST)≤1.5×ULN or ≤3× ULN if liver metastases present

10. Females of childbearing potential must have a negative serum or urine beta human chorionic gonadotrophin (β-hCG) pregnancy test result within 7 days prior to the first dose of LV and must agree to use 2 effective contraceptive methods during the study and for an extended time after the last dose of study drug. Examples of effective contraception methods include, but are not limited to, the following: non-hormonal intrauterine device (IUD), condoms, diaphragm, tubal ligation (injections, implants), barrier methods, vasectomy (for male partners), or complete abstinence. Barrier methods include male and female condoms, diaphragms, and spermicides (creams or gels that contain a chemical to kill sperm).

11. Patients must provide written informed consent.

### Exclusion Criteria

1. Pre-existing neuropathy of ≥Grade 2.

2. History of another primary invasive malignancy that has not been in remission for at least 3 years with the exception of carcinoma in situ of the cervix, squamous or basal cell skin cancer, or thyroid cancer.

3. Known or suspected cerebral/meningeal metastasis that has not been definitively treated.

4. Any active Grade 3 or higher (per the NCI CTCAE v4.03) viral, bacterial, or fungal infection within 2 weeks prior to the first dose of LV.

5. Positive for hepatitis B by surface antigen expression, active hepatitis C infection (positive by polymerase chain reaction (PCR) or on antiviral therapy for hepatitis C within the last 6 months), or a known history of being seropositive for HIV.

6. Documented history of a cerebral vascular event (stroke or transient ischemic attack), unstable angina, myocardial infarction, or cardiac symptoms (including congestive heart failure) consistent with New York Heart Association Class within 6 months prior to their first dose of LV.

7. Females who are breastfeeding.

8. Known hypersensitivity to any excipient contained in the drug formulation of LV.

9. Major surgery ≤3 weeks of study treatment.

10. Prior treatment with LV or prior treatment with an MMAE-containing therapy.

### Discontinuation of Study Drug

A patient's treatment with study drug may be discontinued for any of the following reasons:
Progressive disease (PD)
AE
Investigator decision, non-AE (rationale for decision must be clearly documented)
Patient decision, non-AE
Study termination by sponsor
other non-AE
   Patient Withdrawal from Study

Any patient may be discontinued from the study for any of the following reasons:
Completed study per protocol
Patient withdrawal of consent
Study termination by sponsor
Lost to follow-up
Death
Other

### Treatments

LV is an ADC consisting of the anti-LIV1A monoclonal antibody hLIV22 conjugated to MMAE, a synthetic analog of the naturally occurring tubulin-binding agent, dolastatin 10.

LV is a sterile, preservative-free, white to off-white lyophilized cake or powder for reconstitution for IV administration. LV is supplied in single-use glass vials. Each drug product vial contains LV for Injection, trehalose, histidine, and polysorbate 80. Drug product vials are labeled with a nominal content of

40 mg/vial. Each vial contains 45 mg of LV. Enough overfill is included to allow for 40 mg of LV to be withdrawn for use.

When reconstituted with 8.8 mL Water for Injection (WFI), US Pharmacopeia (USP), the concentration of reconstituted LV product is 5 mg/mL. The reconstituted drug product is a clear to slightly opalescent, colorless to light yellow solution with no visible particulate matter. The pH is approximately 6.0. The reconstituted solution is subsequently diluted in sterile 0.9% Sodium Chloride for Injection, USP, for IV administration.

Single-use vials containing LV must be stored under refrigeration at 2-8° C. in an appropriate locked room accessible only to the pharmacist, investigator, or a duly designated person.

Chemical and physical stability of the reconstituted drug product has been demonstrated for 24 hours at 2-8° C. and at room temperature. However, LV drug product does not contain preservatives; therefore, from a microbiological standpoint, opened and reconstituted vials should be used immediately. If not used immediately, the in-use storage should not be longer than 24 hours under refrigeration at 2-8° C. The prepared dosing solution (reconstituted drug product solution and saline dilution in an IV bag or polypropylene syringe) should be administered within 8 hours after exposing to ambient temperature and light condition.

It is recommended that the drug product vials and solutions be protected from direct sunlight until the time of use.

Reconstituted LV should not be shaken.

Any partially used vials or prepared dosing solutions should be discarded by the site according to institutional drug disposal procedures. Unused vials may only be discarded by the site after authorization by the sponsor.

### Dose and Administration

Dosing is based on patient actual body weight. Doses must be adjusted for patients who experience a ≥10% change in weight from baseline. Other dose adjustments for changes in body weight are permitted per institutional standard.

LV will be given on Day 1, Day 8, and Day 15 in every 3-week cycle. At least 7 days must elapse between administrations of LV. If there is an AE which prevents dosing or lab criteria for dosing are not met on dosing days, the dose should be skipped. Dosing may resume when the AE improves sufficiently to meet dosing criteria or returns to baseline.

In instances where the delay in dosing is not related to the study drug, continued therapy will be discussed with the Medical Monitor on a case-by-case basis.

Infusion-related reactions may occur during the infusion of study treatment. The infusion should be administered at a site properly equipped and staffed to manage anaphylaxis should it occur. Routine premedication should not be administered for the prevention of infusion-related reactions prior to the first dose of LV.

All supportive measures consistent with optimal patient care should be given throughout the study according to institutional standards. Supportive measures may include extending the infusion time and/or administering medications for infusion-related reactions.

Patients who have experienced a Grade 1 or Grade 2 infusion-related reaction with

LV should be premedicated for subsequent infusions.

Patients who experience a Grade 3 infusion-related reaction may potentially receive additional treatment with LV at the discretion of the investigator after discussion with the sponsor.

Premedication may include acetaminophen, an antihistamine, and a corticosteroid administered 30-60 minutes prior to each infusion or according to institutional standards.

If anaphylaxis or a Grade 4 infusion-related reaction occurs, LV administration should be immediately and permanently discontinued.

### Response/Efficacy Assessments

Treatment response will be assessed by radiographic tumor evaluation at protocol-specified time points. Spiral CT or MRI scans of chest, abdomen, and pelvis must be obtained; a CT or MRI of the neck must also be obtained if documented or suspected involvement in this region. The same modality should be used for all subsequent response assessments whenever possible. A diagnostic quality CT is required unless medically contraindicated. For patients unable to tolerate contrast-enhanced CT assessments, MM imaging is acceptable. If any other radiographic or disease assessment exam is conducted per SOC, the assessment information will be collected in the CRF.

Clinical response will be determined by the investigator at each assessment according to RECIST Version 1.1 (Eisenhauer E A et al. Eur. J. Cancer 45(2):228-47 2009). Clinical response may also be assessed by BICR according to RECIST v1.1. In addition, clinical progression per investigator judgment will also be collected in the CRF. In cases of clinical progression, radiologic assessment should be performed to also document radiologic progression. Treatment decisions should be based on investigator assessment.

Patients' clinical data must be available for CRF source verification. Copies of tumor images must be made available for review by the sponsor (or its designee) upon request.

### Example 20: Safety and Tolerability of Ladiratuzumab Vedotin in Patients with Metastatic Breast Cancer

Patients were enrolled in the study described in Example 19. Patients had either first or second line endocrine refractory HR+/HER2− metastatic breast cancer or second line metastatic triple negative breast cancer (TNBC). LV was administered at a dose of 1.0 mg/kg, 1.25 mg/kg, or 1.5 mg/kg by intravenous infusion over the course of 30 minutes on Day 1, Day 8, and Day 15 in every 3-week cycle as described in Example 19.

Subject demographics are shown in the following table:

| | HR+ | | TNBC | | Total | |
|---|---|---|---|---|---|---|
| | (n = 22) | | (n = 18) | | (n = 40) | |
| Age (years) | | | | | | |
| Median | 55 | (37-75) | 54 | (40-74) | 54 | (37-75) |
| Weight (kg) | | | | | | |
| Median | 78 | (54-95) | 73 | (56-113) | 77 | (54-113) |
| ECOG | | | | | | |
| 0 | 15 | (68%) | 11 | (61%) | 26 | (65%) |
| 1 | 7 | (32%) | 7 | (39%) | 14 | (35%) |
| Prior lines of | | | | | | |
| systemic cytotoxic | | | | | | |
| therapy for LA/mTNBC | | | | | | |
| Median | 1 | (0-1) | 1 | (1-1) | 1 | (1-1) |

| LV treatment dose | | | | |
|---|---|---|---|---|
| 1.0 | mg/kg | 10 | 10 | 20 |
| 1.25 | mg/kg | 10 | 8 | 18 |
| 1.5 | mg/kg | 2 | 0 | 2 |

There were no dose limiting toxicities observed at:
Dose level 0 (1 mg/kg Q1W)
Dose level +1 (1.25 mg/kg Q1W)
Dose level +2 (1.5 mg/kg Q1W)

The following table lists the Grade 3 or higher treatment emergent adverse events (TEAEs) that were observed:

| | 1.0 mg/kg | | 1.25 mg/kg | | 1.5 mg/kg | | | |
|---|---|---|---|---|---|---|---|---|
| | (n = 20) | | (n = 18) | | (n = 2) | | Total | |
| Adverse Event | n (%) | | n (%) | | n (%) | | (n = 40) | |
| Any Adverse Event | 12 | (60%) | 14 | (78%) | 2 | (100%) | 28 | (70%) |
| Neutropenia | 2 | (10%) | 4 | (22%) | 1 | (50%) | 7 | (18%) |

| | | | | | | | |
|---|---|---|---|---|---|---|---|
| Fatigue | 4 | (20%) | 2 | (11%) | — | 6 | (15%) |
| Hyperglycemia | 3 | (15%) | 1 | (6%) | — | 4 | (10%) |
| Hypophosphataemia | 1 | (5%) | 2 | (11%) | — | 3 | (8%) |
| Diarrhoea | 1 | (5%) | 1 | (6%) | — | 2 | (5%) |

| | | | | | | | |
|---|---|---|---|---|---|---|---|
| Myalgia | 0 | 1 | (6%) | 1 | (50%) | 2 | (5%) |

| | | | | | | | |
|---|---|---|---|---|---|---|---|
| Nausea | 1 | (5%) | 1 | (6%) | — | 2 | (5%) |
| Neutrophil count | 1 | (5%) | 1 | (6%) | — | 2 | (5%) |
| decreased | | | | | | | |
| Peripheral Sensory | 3 | (15%) | 6 | (33%) | — | 9 | (23%) |
| Neuropathy | | | | | | | |

* Grade 5 ischemic colitis was observed in 1 subject treated with 1 mg/kg LV. This was unrelated to treatment.

The following table lists serious adverse events (SAEs) that were observed:

| | 1.0 mg/kg | | 1.25 mg/kg | | 1.5 mg/kg | | | |
|---|---|---|---|---|---|---|---|---|
| | (n = 20) | | (n = 18) | | (n = 2) | | Total | |
| Adverse Event | n (%) | | n (%) | | n (%) | | (n = 40) | |
| Any Adverse Event | 5 | (25%) | 5 | (28%) | 1 | (50%) | 11 | (28%) |

| | | | | | | | |
|---|---|---|---|---|---|---|---|
| Dyspnoea | 2 | (10%) | 1 | (6%) | 0 | 3 | (8%) |
| Hyperglycaemia | 1 | (5%) | 1 | (6%) | 0 | 2 | (5%) |

| | | | | | | |
|---|---|---|---|---|---|---|
| Pneumonia | 0 | 2 | (11%) | 0 | 2 | (5.%) |

| | | | | | | |
|---|---|---|---|---|---|---|
| Anaemia | 1 | (5%) | 0 | 0 | 1 | (3%) |
| Colitis | 1 | (5%) | 0 | 0 | 1 | (3%) |
| Dehydration | 1 | (5%) | 0 | 0 | 1 | (3%) |
| Diabetic | 1 | (5%) | 0 | 0 | 1 | (3%) |
| ketoacidosis | | | | | | |
| Embolism | 1 | (5%) | 0 | 0 | 1 | (3%) |
| Fatigue | 1 | (5%) | 0 | 0 | 1 | (3%) |
| Febrile neutropenia | 1 | (5%) | 0 | 0 | 1 | (3%) |

The following table shows a comparison the incidence of patients treated with LV using the previous dosing regimen of 2.5 mg/kg on Day 1 of each 21-day cycle (Q3W) compared to the Q1W dosing regimen described in the present example and Example 19:

| | LV 2.5 mg/kg Q3W | LV Q1W | |
|---|---|---|---|
| Adverse Event | n = 65 | N = 40 | |
| Fatigue | 38 (59%) | 18 | (45%) |
| Constipation | 32 (49%) | 10 | (25%) |
| Nausea | 32 (49%) | 14 | (35%) |
| Decreased Appetite | 29 (45%) | 11 | (28%) |
| Diarrhea | 23 (35%) | 11 | (28%) |
| Alopecia | 22 (34%) | 10 | (25%) |
| Peripheral Sensory | 21 (32%) | 9 | (23%) |
| Neuropathy | | | |
| Neutropenia | 19 (29%) | 10 | (25%) |
| Abdominal Pain | 18 (28%) | 7 | (18%) |
| Hypokalemia | 17 (26%) | 4 | (10%) |
| Vomiting | 14 (22%) | 8 | (20%) |

Surprisingly, despite receiving a higher total dose of LV in each 3 week period (3.0-4.5 mg/kg on Q1W vs. 2.5 mg/kg on Q3W), subjects receiving LV once every week experienced a lower incidence of a variety of different adverse event.

### Example 21: A Phase II Study of Ladiratuzumab Vedotin in Advanced Solid Tumors

Ladiratuzumab vedotin (LV) is an antibody-drug conjugate comprising a LIV1 targeted human monoclonal immunoglobulin conjugated via a protease-cleavable valine citrulline linker to the drug monomethyl auristatin E (MMAE), a dolastatin 10 analog. Dolastatins and auristatins belong to a class of chemotherapies that act as microtubule disrupting agents.

This study evaluates the efficacy, safety and tolerability of 2.5 mg/kg ladiratuzumab vedotin in patients with locally advanced or metastatic small cell lung cancer, non-small cell lung cancer, head and neck cancer, esophageal cancer, gastric cancer or gastroesophageal junction cancer. Patients with locally-advanced or metastatic small cell lung cancer, non-small cell lung cancer, head and neck cancer, esophageal cancer, gastric cancer or gastroesophageal junction cancer whose disease has progressed after first and subsequent lines of treatment have significant unmet medical need for therapies that can meaningfully improve their prognosis.

### Methods

This global, open label, multicenter trial is designed to assess the safety, tolerability, and activity of ladiratuzumab vedotin for the treatment of selected solid tumors. Eligible patients are at least 18 years of age with inoperable, locally advanced or metastatic cancer. Patients are enrolled into one of 6 cohorts based on tumor type, including small cell lung cancer, non-small cell lung cancer-squamous, non-small cell lung cancer-nonsquamous, head and neck squamous cell carcinoma, esophageal squamous cell carcinoma, and gastric and gastroesophageal junction adenocarcinoma.

In all eligible patients, ladiratuzumab vedotin is administered at a dose of 2.5 mg/kg as a 30 minute intravenous infusion on Day 1 of each 21-day cycle (Q3W). Dosing is based on the subject's body weight, measured each cycle. For subjects weighing >100 kg, dosing will be based on a 100 kg maximum weight (calculated to not exceed 250 mg per cycle). An individual's dose may be modified based upon treatment-related adverse events. Response is assessed every 6 weeks (±3 days) for the first 12 months and every 12 weeks (±7 days) thereafter. RECIST v1.1 is used by the investigator to score responses for primary and secondary endpoints as well as progression. Objective responses are confirmed with repeat scans 4-6 weeks after the first documentation of response.

The primary analysis of the study will be performed separately for each cohort when all treated subjects in the cohort have been followed for at least 6 months or come off study, whichever comes first. The primary efficacy endpoint of confirmed ORR per RECIST v1.1 will be estimated for each cohort based on the full analysis set (FAS), comprising all subjects who received any amount of study treatment. The point estimate of ORR and 90% exact confidence intervals (CIs) using the Clopper-Pearson method will be provided for each cohort.

Interim futility analyses will be performed separately for each cohort after at least 12 subjects of a given cohort have been treated and are efficacy evaluable post-baseline. The Bayesian predictive probability approach will be used to determine the futility criteria. At the time of each interim analysis, the predictive probability of success (PPoS) will be calculated. A PPoS <10% indicates that it is unlikely the ORR will be better than the response rate of current standard of care at the end of the study given the interim result. Based on efficacy and safety data, together with the PPoS, a cohort may be stopped early by the sponsor.

Inclusion criteria and exclusion criteria for patients enrolled in trial are shown in Table 3.

TABLE 3
List of inclusion and exclusion criteria

| Inclusion | 1. Age of at least 18 years, or legal age according to local regulations, | |
|---|---|---|
| Criteria | whichever is older | |
| | 2. Measurable disease according to RECIST v1.1 as assessed by the | |
| | investigator | |
| | | A minimum of one non-nodal lesion ≥10 mm in the longest diameter |
| | | from a non-irradiated area; or |
| | | Lymph node lesion ≥15 mm in the shortest diameter from a non- |
| | | irradiated area |
| | | If target lesion(s) are located within previously irradiated area only, the |
| | | subject can be enrolled only if there has been demonstrated progression |
| | | in the “in field” lesion and upon approval of the sponsor's medical |
| | | monitor |
| | 3. Eastern Cooperative Oncology Group (ECOG) Performance Score of 0 or 1 | |
| | 4. The following baseline laboratory data: | |
| | | absolute neutrophil count (ANC) ≥1500/μL assessed at least 2 weeks |
| | | after growth factor support, if applicable |
| | | platelet count ≥100 × 10⁹/L assessed at least 2 weeks after transfusion |
| | | with blood products, if applicable |
| | | hemoglobin (≥8.0 g/dL) assessed at least 2 weeks after transfusion with |
| | | blood products and/or growth factor support, if applicable |
| | | serum bilirubin ≤1.5 × upper limit of normal (ULN) or direct bilirubin |
| | | ≤3 × ULN in subjects diagnosed with Gilbert's syndrome |
| | | estimated glomerular filtration rate (GFR) ≥30 mL/min/1.73 m2 using |
| | | the Modification of Diet in Renal Disease (MDRD) study equation as |
| | | applicable |
| | | alanine aminotransferase (ALT) and aspartate aminotransferase (AST) |
| | | ≤1.5 × ULN (if liver metastases are present, then ≤3 × ULN is allowed) |
| | 5. Subjects of childbearing potential as defined in Section Error! Reference | |
| | source not found., under the following conditions: | |
| | a. | Must have a negative serum or urine pregnancy test (minimum |
| | | sensitivity 25 mIU/mL or equivalent units of beta human chorionic |
| | | gonadotropin [β-hCG]) result within 3 days prior to the first dose of LV. |
| | | Subjects with false positive results and documented verification that the |
| | | subject is not pregnant are eligible for participation. |
| | b. | Must agree not to try to become pregnant during the study and for at |
| | | least 6 months after the final dose of study drug administration |
| | c. | Must agree not to breastfeed or donate ova, starting at time of informed |
| | | consent and continuing through 6 months after the final dose of study |
| | | drug administration |
| | d. | If sexually active in a way that could lead to pregnancy, must |
| | | consistently use 2 highly effective methods of birth control starting at |
| | | time of informed consent and continuing throughout the study and for at |
| | | least 6 months after the final dose of study drug administration |
| | 6. Subjects who can father children, under the following conditions: | |
| | a. | Must agree not to donate sperm starting at time of informed consent and |
| | | continuing throughout the study period and for at least 6 months after |
| | | the final study drug administration |
| | b. | If sexually active with a subject of childbearing potential in a way that |
| | | could lead to pregnancy, must consistently use 2 highly effective |
| | | methods of birth control starting at time of informed consent and |
| | | continuing throughout the study and for at least 6 months after the final |
| | | dose of study drug administration |
| | c. | If sexually active with a subject who is pregnant or breastfeeding, must |
| | | consistently use one of 2 contraception options starting at time of |
| | | informed consent and continuing throughout the study and for at least |
| | | 6 months after the final dose of study drug administration |

```
7. Available and adequate archival baseline tumor sample is required. If an
  archival baseline tumor sample is not available, a fresh biopsy sample may
  be submitted if medically feasible or the medical monitor should be
  contacted to review this requirement. Formalin fixed paraffin embedded
  (FFPE) blocks and core needle or excisional biopsy of metastatic site are
  preferred.
8. Relapsed, locally advance or metastatic small cell lung cancer, non-small cell
lung cancer-squamous, non-small cell lung cancer-nonsquamous, head and
neck squamous cell carcinoma, esophageal squamous cell carcinoma, and
gastric and gastroesophageal junction adenocarcinoma that has failed prior lines
of systemic treatment as specified and which are not candidates for standard
therapy.
```

```
Small Cell Lung Cancer
  Subjects must have pathologically-documented SCLC
  Must have extensive stage disease
  Must have disease progression during or following prior platinum-
based systemic chemotherapy for extensive stage disease
  No more than 1 prior line of cytotoxic chemotherapy for extensive
disease stage
  May have received prior anti-PD(L)1 therapy, unless contraindicated
  Mixed SCLC/neuroendocrine tumors with NSCLC histologies are not
eligible
Non-Small Cell Lung Cancer- Squamous
  Must have pathologically-documented squamous cell NSCLC
  Must have unresectable locally advanced or metastatic disease
  Must have disease progression during or following systemic therapy
  a. Participants must have progressed during or after a platinum-
  based combination therapy administered for the treatment of
  metastatic disease
  b. Participants must have progressed within 6 months of last
  dose of platinum-based adjuvant, neoadjuvant, or definitive
  chemotherapy, or concomitant chemoradiation regimen for early
  stage or locally advanced stage disease.
  Subjects with known epidermal growth factor receptor (EGFR),
anaplastic lymphoma kinase (ALK), reactive oxygen species (ROS),
BRAF, or other actionable mutations are not eligible
  No more than 1 prior line of cytotoxic chemotherapy for their
advanced disease
  Must have received prior anti-PD(L)1 therapy, unless contraindicated
  Subjects with mixed histology NSCLC are eligible as long as the
tumor is predominantly squamous histology. Mixed
SCLC/neuroendocrine tumors with NSCLC histologies are not eligible.
Non-Small Cell Lung Cancer- Nonsquamous
  Must have pathologically-documented nonsquamous NSCLC
  Must have unresectable locally advanced or metastatic disease
  Must have disease progression during or following systemic therapy
  a. Participants must have progressed during or after a platinum-
  based combination therapy administered for the treatment of
  metastatic disease
  b. Participants must have progressed within 6 months of last
  dose of platinum-based adjuvant, neoadjuvant, or definitive
  chemotherapy, or concomitant chemoradiation regimen for early
  stage or locally advanced state disease.
  Subjects with known EGFR, ALK, ROS, BRAF, tropomyosin receptor
kinase (TRK), or other actionable mutations are not eligible
  Must have had prior platinum-based chemotherapy
  No more than 1 prior line of cytotoxic chemotherapy for their
advanced disease
  Must have received prior anti-PD(L)1 therapy, unless contraindicated
  Mixed tumors will be categorized by the predominant cell type.
Subject is ineligible if the subject has predominantly squamous cell
histology NSCLC or if small cell elements are present.
Head and Neck Squamous Cell Carcinoma
  Must have pathologically-documented squamous cell carcinoma of the
head and neck with primary tumor site arising from the oral cavity,
oropharynx, hypopharynx, and larynx; tumors arising from the
nasopharynx are excluded
  Must have unresectable locally recurrent or metastatic disease
  Must have disease progression during or following prior line of
systemic therapy
  a. Disease progression after treatment with a platinum-
  containing regimen for recurrent/metastatic disease; or
  b. Recurrence/progression within 6 months of last dose of
  platinum therapy given as part of a multimodal therapy in the
  curative setting
  No more than 1 line of cytotoxic chemotherapy for their advanced
disease
  May have received prior anti-PD(L)1 therapy, unless contraindicated
Esophageal Squamous Cell Carcinoma
  Must have pathologically-documented squamous cell carcinoma of the
esophagus
  Must have unresectable locally advanced or metastatic disease
  Must have disease progression during or following systemic therapy
  Must have received prior platinum-based chemotherapy
  No more than 1 line of cytotoxic chemotherapy for their advanced
disease
Gastric and Gastroesophageal Junction Adenocarcinoma
  Must have pathologically-documented gastric or GEJ adenocarcinoma
  Must have unresectable locally advanced or metastatic disease
  Must have had prior platinum-based therapy
  Must have disease progression during or following systemic therapy
  Participants with known human epidermal growth factor receptor 2
(HER2) overexpression must have received prior HER2-targeted
therapy
  No more than 1 line of prior cytotoxic chemotherapy for their
advanced disease
  Subjects with known deficient mismatch repair (dMMR) and/or
microsatellite instability-high (MSI-H) may have received prior anti-
PD(L)1 therapy, unless contraindicated
```

| | |
|---|---|
| Exclusion | 1. Active concurrent malignancy or a previous malignancy within the past 3 years |
| Criteria | 2. Known active central nervous system lesions |
| | 3. Any ongoing clinically significant toxicity associated with prior treatment |
| | (Grade 2 or higher) |
| | 4. Ongoing sensory or motor neuropathy of Grade ≥2 |
| | 5. Has received prior radiotherapy within 2 weeks of start of study treatment |

LV is a sterile, preservative-free, white to off-white lyophilized cake or powder for reconstitution for IV administration. LV is supplied in single-use glass vials. Each drug product vial contains LV for injection, trehalose, histidine, and polysorbate 80. Drug product vials are labeled with a nominal content of 40 mg/vial. Each vial contains 45 mg of LV. Enough overfill is included to allow for 40 mg of LV to be withdrawn for use.

When reconstituted with 8.8 mL water for injection (WFI), United States Pharmacopeia (USP) grade or equivalent, the concentration of reconstituted LV product is 5 mg/mL. The reconstituted drug product is a clear to slightly opalescent, colorless to light yellow solution with no visible particulate matter. The pH is approximately 6.0. The reconstituted solution is subsequently diluted in sterile 0.9% Sodium Chloride for Injection, USP grade or equivalent, for IV administration.

Dose modifications for LV treatment-associated toxicity are described in Table 4. The maximum doses after modifications are:
200 mg for subjects reduced to 2.0 mg/kg
150 mg for subjects reduced to 1.5 mg/kg

TABLE 4
Dose levels

| Dose Level | Dose | Maximum Dose |
|---|---|---|
| Starting dose | 2.5 mg/kg | 250 mg |
| −1 | 2.0 mg/kg | 200 mg |
| −2 | 1.5 mg/kg | 150 mg |

Doses reduced for LV-related toxicity should not be re-escalated.

If a subject has a clinically significant, unresolved AE on Day 1 of Cycle 2 (C2) or beyond, the start of the cycle may be delayed for up to 14 days. Delays of >14 days must be approved by the medical monitor.

In the event a subject is unable to tolerate their dose level, additional treatment cycles (C2 or later) may be administered at a lower dose level upon approval by the medical monitor.

Objectives and endpoints are described in Table 5. Confirmed Objective Response Rate (ORR) is defined as the proportion of subjects who achieve a confirmed Complete Response (CR) or Partial Response (PR) according to RECIST v1.1 as assessed by the investigator. Subjects who do not have at least 2 post-baseline response assessments (initial response and confirmation scan) will be counted as non-responders.

Disease Control Rate (DCR) is defined as the proportion of subjects who achieve a confirmed CR or PR according to RECIST v1.1 as assessed by the investigator, or meet the SD criteria at least once after start of study treatment at a minimum interval of 6 weeks. Subjects who do not have at least 1 post-baseline response assessment will be counted as non-responders.

Duration of Response (DOR) is defined as the time from the first documentation of objective response (CR or PR that is subsequently confirmed) to the first documentation of PD or death due to any cause, whichever comes first.

DOR data will be censored as described below:
Subjects who do not have PD and are still on study at the time of an analysis will be censored at the date of last disease assessment documenting absence of PD.
Subjects who started a new anticancer treatment prior to documentation of PD will be censored at the date of last disease assessment prior to the start of new treatment.
Subjects who are removed from the study prior to documentation of PD will be censored at the date of last disease assessment documenting absence of PD.

DOR will only be calculated for subjects who achieve a confirmed CR or PR.

Progression-free survival (PFS) is defined as the time from the start of study treatment to the first documentation of PD or death due to any cause, whichever comes first.

The same censoring rules as for DOR will be applied to PFS. Subjects lacking an evaluation of tumor response after their first dose of study drug will have their event time censored at Day 1.

Overall Survival (OS) is defined as the time from the start of study treatment to date of death due to any cause. In the absence of death, survival time will be censored at the last date the subject is known to be alive (i.e., date of last contact).

TABLE 5
Objectives and endpoints

| Primary Objective | Corresponding Primary Endpoint |
|---|---|
| Evaluate antitumor activity of LV | Investigator-determined confirmed ORR as |
| | measured by RECIST v1.1 |
| Secondary Objectives | Corresponding Secondary Endpoints |
| Evaluate the safety and tolerability of LV | Type, incidence, severity, seriousness, and |
| | relatedness of AEs |
| Evaluate stability and control of disease | Investigator-determined DCR as measured by |
| | RECIST v1.1 |
| Evaluate durability of response in subjects | Investigator-determined DOR as measured by |
| who respond to LV | RECIST v1.1 |
| Evaluate PFS of subjects treated with LV | Investigator-determined PFS as measured by |
| | RECIST v1.1 |
| Evaluate survival of subjects treated with LV | OS |
| Assess PK of LV | Selected PK parameters for LV, total antibody, |
| | and MMAE |
| Assess immunogenicity of LV | Incidence of ATAs to LV |
| Additional Objectives | Corresponding Additional Endpoints |
| Assess biomarkers of biological activity and | Relationship between biomarkers in blood and |
| resistance and predictive biomarkers of response | tumor tissue to efficacy, safety, or other |
| | biomarker endpoints following treatment with LV |

### Example 22: Anti-Tumor Activity of Ladiratuzumab Vedotin in Patients with Small Cell Lung Cancer

Human patients with unresectable, locally advanced or metastatic small cell lung cancer will be treated with ladiratuzumab vedotin (LV). Patients will have cancers that express LIV1. Patients will have extensive disease stage and will have disease progression during or following prior platinum-based systemic chemotherapy for extensive stage disease. Patients will have no more than 1 prior line of cytotoxic chemotherapy for the extensive disease stage. Patients may have received prior anti-PD(L)1 therapy, unless contraindicated. Patients will not have mixed SCLC/neuroendocrine tumors with NSCLC histologies.

LV will be administered by intravenous (IV) infusion at a dose of 2.5 mg/kg on Day 1 of each 21-day cycle. Dosing may not exceed 250 mg per infusion. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 2.5 mg/kg as a 30 minute IV infusion every 21 days. Any subject receiving >200 mg LV per cycle (weight >80 kg) is required to receive prophylactic granulocyte colony-stimulating factor (G-CSF). For patients weighing more than 100 kg, dosing will be capped at 250 mg per infusion.

### Example 23: Anti-Tumor Activity of Ladiratuzumab Vedotin in Patients with Squamous Non-Small Cell Lung Cancer

Human patients with unresectable, locally advanced or metastatic squamous non-small cell lung cancer will be treated with ladiratuzumab vedotin (LV). Patients will have cancers that express LIV1. Patients will have extensive disease stage and will have disease progression during or following prior platinum-based systemic chemotherapy for extensive stage disease. Patients will have no more than 1 prior line of cytotoxic chemotherapy for the extensive disease stage. Patients may have received prior anti-PD(L)1 therapy, unless contraindicated. Patients may have mixed histology NSCLC as long as the tumor is predominantly squamous histology. Patients will not have mixed SCLC/neuroendocrine tumors with NSCLC histologies. Patients will not have known epidermal growth factor receptor (EGFR), anaplastic lymphoma kinase (ALK), reactive oxygen species (ROS), BRAF, or other actionable mutations.

LV will be administered by intravenous (IV) infusion at a dose of 2.5 mg/kg on Day 1 of each 21-day cycle. Dosing may not exceed 250 mg per infusion. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 2.5 mg/kg as a 30 minute IV infusion every 21 days. Any subject receiving >200 mg LV per cycle (weight >80 kg) is required to receive prophylactic granulocyte colony-stimulating factor (G-CSF). For patients weighing more than 100 kg, dosing will be capped at 250 mg per infusion.

### Example 24: Anti-Tumor Activity of Ladiratuzumab Vedotin in Patients with Nonsquamous Non-Small Cell Lung Cancer

Human patients with unresectable, locally advanced or metastatic nonsquamous non-small cell lung cancer will be treated with ladiratuzumab vedotin (LV). Patients will have cancers that express LIV1. Patients will have disease progression during or following prior platinum-based systemic chemotherapy for extensive stage disease. Patients will have no more than 1 prior line of cytotoxic chemotherapy for their advanced disease. Patients may have received prior anti-PD(L)1 therapy, unless contraindicated. Patients will not have predominantly squamous cell histology NSCLC or small cell elements. Patients will not have known EGFR, ALK, ROS, BRAF, tropomyosin receptor kinase (TRK), or other actionable mutations.

LV will be administered by intravenous (IV) infusion at a dose of 2.5 mg/kg on Day 1 of each 21-day cycle. Dosing may not exceed 250 mg per infusion. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 2.5 mg/kg as a 30 minute IV infusion every 21 days. Any subject receiving >200 mg LV per cycle (weight >80 kg) is required to receive prophylactic granulocyte colony-stimulating factor (G-CSF). For patients weighing more than 100 kg, dosing will be capped at 250 mg per infusion.

### Example 25: Anti-Tumor Activity of Ladiratuzumab Vedotin in Patients with Head and Neck Squamous Cell Carcinoma

Human patients with unresectable, locally advanced or metastatic head and neck squamous cell carcinoma will be treated with ladiratuzumab vedotin (LV). Patients will have cancers that express LIV1. Patients will have squamous cell carcinoma of the head and neck with primary tumor site arising from the oral cavity, oropharynx, hypopharynx, or larynx. Patients will have extensive disease stage and will have disease progression during or following prior platinum-based systemic chemotherapy for extensive stage disease. Patients will have no more than 1 prior line of cytotoxic chemotherapy for the extensive disease stage. Patients may have received prior anti-PD(L)1 therapy, unless contraindicated.

LV will be administered by intravenous (IV) infusion at a dose of 2.5 mg/kg on Day 1 of each 21-day cycle. Dosing may not exceed 250 mg per infusion. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 2.5 mg/kg as a 30 minute IV infusion every 21 days. Any subject receiving >200 mg LV per cycle (weight >80 kg) is required to receive prophylactic granulocyte colony-stimulating factor (G-CSF). For patients weighing more than 100 kg, dosing will be capped at 250 mg per infusion.

### Example 26: Anti-Tumor Activity of Ladiratuzumab Vedotin in Patients with Esophageal Squamous Cell Carcinoma

Human patients with unresectable, locally advanced or metastatic esophageal squamous cell carcinoma will be treated with ladiratuzumab vedotin (LV). Patients will have cancers that express LIV1. Patients will have extensive disease stage and will have disease progression during or following prior systemic therapy. Patients will have had prior platinum-based chemotherapy. Patients will have no more than 1 prior line of cytotoxic chemotherapy for the extensive disease stage.

LV will be administered by intravenous (IV) infusion at a dose of 2.5 mg/kg on Day 1 of each 21-day cycle. Dosing may not exceed 250 mg per infusion. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 2.5 mg/kg as a 30 minute IV infusion every 21 days. Any subject receiving >200 mg LV per cycle (weight >80 kg) is required to receive prophylactic granulocyte colony-stimulating factor (G-CSF). For patients weighing more than 100 kg, dosing will be capped at 250 mg per infusion.

### Example 27: Anti-Tumor Activity of Ladiratuzumab Vedotin in Patients with Gastric or Gastroesophageal Adenocarcinoma

Human patients with unresectable, locally advanced or metastatic gastric or gastroesophageal adenocarcinoma will be treated with ladiratuzumab vedotin (LV). Patients will have cancers that express LIV1. Patients will have extensive disease stage and will have disease progression during or following prior systemic therapy. Patients will have had prior platinum-based chemotherapy. Patients will have no more than 1 prior line of cytotoxic chemotherapy for the extensive disease stage. Patients with human epidermal growth factor receptor 2 (HER2) will have received prior HER2-targeted therapy. Patients with deficient mismatch repair (dMMR) and/or microsatellite instability-high (MSI-H) may have received prior anti-PD(L)1 therapy, unless contraindicated.

LV will be administered by intravenous (IV) infusion at a dose of 2.5 mg/kg on Day 1 of each 21-day cycle. Dosing may not exceed 250 mg per infusion. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 2.5 mg/kg as a 30 minute IV infusion every 21 days. Any subject receiving >200 mg LV per cycle (weight >80 kg) is required to receive prophylactic granulocyte colony-stimulating factor (G-CSF). For patients weighing more than 100 kg, dosing will be capped at 250 mg per infusion.

### Example 28: A Phase II Study of Ladiratuzumab Vedotin in Advanced Solid Tumors

Ladiratuzumab vedotin (LV) is an antibody-drug conjugate comprising a LIV1 targeted human monoclonal immunoglobulin conjugated via a protease-cleavable valine citrulline linker to the drug monomethyl auristatin E (MMAE), a dolastatin 10 analog. Dolastatins and auristatins belong to a class of chemotherapies that act as microtubule disrupting agents.

This study evaluates the efficacy, safety and tolerability of 1.0 mg/kg or 1.25 mg/kg ladiratuzumab vedotin in patients with locally advanced or metastatic small cell lung cancer, non-small cell lung cancer, head and neck cancer, esophageal cancer, gastric cancer or gastroesophageal junction cancer. Patients with locally-advanced or metastatic small cell lung cancer, non-small cell lung cancer, head and neck cancer, esophageal cancer, gastric cancer or gastroesophageal junction cancer whose disease has progressed after first and subsequent lines of treatment have significant unmet medical need for therapies that can meaningfully improve their prognosis.

### Methods

This global, open label, multicenter trial is designed to assess the safety, tolerability, and activity of ladiratuzumab vedotin for the treatment of selected solid tumors. Eligible patients are at least 18 years of age with inoperable, locally advanced or metastatic cancer. Patients are enrolled into one of 6 cohorts based on tumor type, including small cell lung cancer, non-small cell lung cancer-squamous, non-small cell lung cancer-nonsquamous, head and neck squamous cell carcinoma, esophageal squamous cell carcinoma, and gastric and gastroesophageal junction adenocarcinoma.

In all eligible patients, ladiratuzumab vedotin is administered at a dose of 1.0 mg/kg or 1.25 mg/kg as a 30 minute intravenous infusion on Day 1 of each 7-day cycle (Q1W). Dosing is based on the subject's body weight, measured each cycle. For subjects weighing >100 kg, dosing will be based on a 100 kg maximum weight (calculated to not exceed 125 mg per cycle). An individual's dose may be modified based upon treatment-related adverse events. Response is assessed every 6 weeks (±3 days) for the first 12 months and every 12 weeks (±7 days) thereafter. RECIST v1.1 is used by the investigator to score responses for primary and secondary endpoints as well as progression. Objective responses are confirmed with repeat scans 4-6 weeks after the first documentation of response.

The primary analysis of the study will be performed separately for each cohort when all treated subjects in the cohort have been followed for at least 6 months or come off study, whichever comes first. The primary efficacy endpoint of confirmed ORR per RECIST v1.1 will be estimated for each cohort based on the full analysis set (FAS), comprising all subjects who received any amount of study treatment. The point estimate of ORR and 90% exact confidence intervals (CIs) using the Clopper-Pearson method will be provided for each cohort.

Interim futility analyses will be performed separately for each cohort after at least 12 subjects of a given cohort have been treated and are efficacy evaluable post-baseline. The Bayesian predictive probability approach will be used to determine the futility criteria. At the time of each interim analysis, the predictive probability of success (PPoS) will be calculated. A PPoS <10% indicates that it is unlikely the ORR will be better than the response rate of current standard of care at the end of the study given the interim result. Based on efficacy and safety data, together with the PPoS, a cohort may be stopped early by the sponsor.

Inclusion criteria and exclusion criteria for patients enrolled in trial are shown in Table 6.

TABLE 6
List of inclusion and exclusion criteria

| Inclusion | 1. Age of at least 18 years, or legal age according to local regulations, | |
|---|---|---|
| Criteria | whichever is older | |
| | 2. Measurable disease according to RECIST v1.1 as assessed by the | |
| | investigator | |
| | | A minimum of one non-nodal lesion ≥10 mm in the longest diameter |
| | | from a non-irradiated area; or |
| | | Lymph node lesion ≥15 mm in the shortest diameter from a non- |
| | | irradiated area |
| | | If target lesion(s) are located within previously irradiated area only, the |
| | | subject can be enrolled only if there has been demonstrated progression |
| | | in the “in field” lesion and upon approval of the sponsor's medical |
| | | monitor |
| | 3. Eastern Cooperative Oncology Group (ECOG) Performance Score of 0 or 1 | |
| | 4. The following baseline laboratory data: | |
| | | absolute neutrophil count (ANC) ≥1500/μL assessed at least 2 weeks |
| | | after growth factor support, if applicable |
| | | platelet count ≥100 × 10⁹/L assessed at least 2 weeks after transfusion |
| | | with blood products, if applicable |
| | | hemoglobin (≥8.0 g/dL) assessed at least 2 weeks after transfusion with |
| | | blood products and/or growth factor support, if applicable |
| | | serum bilirubin ≤1.5 × upper limit of normal (ULN) or direct bilirubin |
| | | ≤3 × ULN in subjects diagnosed with Gilbert's syndrome |
| | | estimated glomerular filtration rate (GFR) ≥30 mL/min/1.73 m2 using |
| | | the Modification of Diet in Renal Disease (MDRD) study equation as |
| | | applicable |
| | | alanine aminotransferase (ALT) and aspartate aminotransferase (AST) |
| | | ≤1.5 × ULN (if liver metastases are present, then ≤3 × ULN is allowed) |
| | 5. Subjects of childbearing potential as defined in Section Error! Reference | |
| | source not found., under the following conditions: | |
| | d. | Must have a negative serum or urine pregnancy test (minimum |
| | | sensitivity 25 mlU/mL or equivalent units of beta human chorionic |
| | | gonadotropin [β-hCG]) result within 3 days prior to the first dose of LV. |
| | | Subjects with false positive results and documented verification that the |
| | | subject is not pregnant are eligible for participation. |
| | e. | Must agree not to try to become pregnant during the study and for at |
| | | least 6 months after the final dose of study drug administration |
| | f. | Must agree not to breastfeed or donate ova, starting at time of informed |
| | | consent and continuing through 6 months after the final dose of study |
| | | drug administration |
| | g. | If sexually active in a way that could lead to pregnancy, must |
| | | consistently use 2 highly effective methods of birth control starting at |
| | | time of informed consent and continuing throughout the study and for at |
| | | least 6 months after the final dose of study drug administration |
| | 6. Subjects who can father children, under the following conditions: | |
| | h. | Must agree not to donate sperm starting at time of informed consent and |
| | | continuing throughout the study period and for at least 6 months after |
| | | the final study drug administration |
| | i. | If sexually active with a subject of childbearing potential in a way that |
| | | could lead to pregnancy, must consistently use 2 highly effective |
| | | methods of birth control starting at time of informed consent and |
| | | continuing throughout the study and for at least 6 months after the final |
| | | dose of study drug administration |
| | j. | If sexually active with a subject who is pregnant or breastfeeding, must |
| | | consistently use one of 2 contraception options starting at time of |
| | | informed consent and continuing throughout the study and for at least |
| | | 6 months after the final dose of study drug administration |

```
7. Available and adequate archival baseline tumor sample is required. If an
  archival baseline tumor sample is not available, a fresh biopsy sample may
  be submitted if medically feasible or the medical monitor should be
  contacted to review this requirement. Formalin fixed paraffin embedded
  (FFPE) blocks and core needle or excisional biopsy of metastatic site are
  preferred.
8. Relapsed, locally advance or metastatic small cell lung cancer, non-small cell
lung cancer-squamous, non-small cell lung cancer-nonsquamous, head and
neck squamous cell carcinoma, esophageal squamous cell carcinoma, and
gastric and gastroesophageal junction adenocarcinoma that has failed prior lines
of systemic treatment as specified and which are not candidates for standard
therapy.
```

```
Small Cell Lung Cancer
  Subjects must have pathologically-documented SCLC
  Must have extensive stage disease
  Must have disease progression during or following prior platinum-
based systemic chemotherapy for extensive stage disease
  No more than 1 prior line of cytotoxic chemotherapy for extensive
disease stage
  May have received prior anti-PD(L)1 therapy, unless contraindicated
  Mixed SCLC/neuroendocrine tumors with NSCLC histologies are not
eligible
Non-Small Cell Lung Cancer- Squamous
  Must have pathologically-documented squamous cell NSCLC
  Must have unresectable locally advanced or metastatic disease
  Must have disease progression during or following systemic therapy
  a. Participants must have progressed during or after a platinum-
  based combination therapy administered for the treatment of
  metastatic disease
  b. Participants must have progressed within 6 months of last
  dose of platinum-based adjuvant, neoadjuvant, or definitive
  chemotherapy, or concomitant chemoradiation regimen for early
  stage or locally advanced stage disease.
  Subjects with known epidermal growth factor receptor (EGFR),
anaplastic lymphoma kinase (ALK), reactive oxygen species (ROS),
BRAF, or other actionable mutations are not eligible
  No more than 1 prior line of cytotoxic chemotherapy for their
advanced disease
  Must have received prior anti-PD(L)1 therapy, unless contraindicated
  Subjects with mixed histology NSCLC are eligible as long as the
tumor is predominantly squamous histology. Mixed
SCLC/neuroendocrine tumors with NSCLC histologies are not eligible.
Non-Small Cell Lung Cancer- Nonsquamous
  Must have pathologically-documented nonsquamous NSCLC
  Must have unresectable locally advanced or metastatic disease
  Must have disease progression during or following systemic therapy
  a. Participants must have progressed during or after a platinum-
  based combination therapy administered for the treatment of
  metastatic disease
  b. Participants must have progressed within 6 months of last
  dose of platinum-based adjuvant, neoadjuvant, or definitive
  chemotherapy, or concomitant chemoradiation regimen for early
  stage or locally advanced state disease.
  Subjects with known EGFR, ALK, ROS, BRAF, tropomyosin receptor
kinase (TRK), or other actionable mutations are not eligible
  Must have had prior platinum-based chemotherapy
  No more than 1 prior line of cytotoxic chemotherapy for their
advanced disease
  Must have received prior anti-PD(L)1 therapy, unless contraindicated
  Mixed tumors will be categorized by the predominant cell type.
Subject is ineligible if the subject has predominantly squamous cell
histology NSCLC or if small cell elements are present.
Head and Neck Squamous Cell Carcinoma
  Must have pathologically-documented squamous cell carcinoma of the
head and neck with primary tumor site arising from the oral cavity,
oropharynx, hypopharynx, and larynx; tumors arising from the
nasopharynx are excluded
  Must have unresectable locally recurrent or metastatic disease
  Must have disease progression during or following prior line of
systemic therapy
  a. Disease progression after treatment with a platinum-
  containing regimen for recurrent/metastatic disease; or
  b. Recurrence/progression within 6 months of last dose of
  platinum therapy given as part of a multimodal therapy in the
  curative setting
  No more than 1 line of cytotoxic chemotherapy for their advanced
disease
  May have received prior anti-PD(L)1 therapy, unless contraindicated
Esophageal Squamous Cell Carcinoma
  Must have pathologically-documented squamous cell carcinoma of the
esophagus
  Must have unresectable locally advanced or metastatic disease
  Must have disease progression during or following systemic therapy
  Must have had prior platinum-based chemotherapy
  No more than 1 line of cytotoxic chemotherapy for their advanced
disease
Gastric and Gastroesophageal Junction Adenocarcinoma
  Must have pathologically-documented gastric or GEJ adenocarcinoma
  Must have unresectable locally advanced or metastatic disease
  Must have received prior platinum-based therapy
  Must have disease progression during or following systemic therapy
  Participants with known human epidermal growth factor receptor 2
(HER2) overexpression must have received prior HER2-targeted
therapy
  No more than 1 line of prior cytotoxic chemotherapy for their
advanced disease
  Subjects with known deficient mismatch repair (dMMR) and/or
microsatellite instability-high (MSI-H) may have received prior anti-
PD(L)1 therapy, unless contraindicated
```

| | |
|---|---|
| Exclusion | 1. Active concurrent malignancy or a previous malignancy within the past 3 |
| Criteria | years |
| | 2. Known active central nervous system lesions |
| | 3. Any ongoing clinically significant toxicity associated with prior treatment |
| | (Grade 2 or higher) |
| | 4. Ongoing sensory or motor neuropathy of Grade ≥2 |
| | 5. Has received prior radiotherapy within 2 weeks of start of study treatment |

LV is a sterile, preservative-free, white to off-white lyophilized cake or powder for reconstitution for IV administration. LV is supplied in single-use glass vials. Each drug product vial contains LV for injection, trehalose, histidine, and polysorbate 80. Drug product vials are labeled with a nominal content of 40 mg/vial. Each vial contains 45 mg of LV. Enough overfill is included to allow for 40 mg of LV to be withdrawn for use.

When reconstituted with 8.8 mL water for injection (WFI), United States Pharmacopeia (USP) grade or equivalent, the concentration of reconstituted LV product is 5 mg/mL. The reconstituted drug product is a clear to slightly opalescent, colorless to light yellow solution with no visible particulate matter. The pH is approximately 6.0. The reconstituted solution is subsequently diluted in sterile 0.9% Sodium Chloride for Injection, USP grade or equivalent, for IV administration.

Dose modifications for LV treatment-associated toxicity are described in Table 7. The maximum doses after modifications are:
100 mg for subjects reduced to 1.0 mg/kg
75 mg for subjects reduced to 0.75 mg/kg

TABLE 7
Dose levels

| | | |
|---|---|---|
| Dose Level | Dose | Maximum Dose |

| | | | | |
|---|---|---|---|---|
| Starting dose | 1.25 | mg/kg | 125 | mg |
| −1 | 1.0 | mg/kg | 100 | mg |
| −2 | 0.75 | mg/kg | 75 | mg |

Doses reduced for LV-related toxicity should not be re-escalated.

If a subject has a clinically significant, unresolved AE on Day 1 of Cycle 2 (C2) or beyond, the start of the cycle may be delayed.

In the event a subject is unable to tolerate their dose level, additional treatment cycles (C2 or later) may be administered at a lower dose level upon approval by the medical monitor.

Objectives and endpoints are described in Table 5. Confirmed Objective Response Rate (ORR) is defined as the proportion of subjects who achieve a confirmed Complete Response (CR) or Partial Response (PR) according to RECIST v1.1 as assessed by the investigator. Subjects who do not have at least 2 post-baseline response assessments (initial response and confirmation scan) will be counted as non-responders.

Disease Control Rate (DCR) is defined as the proportion of subjects who achieve a confirmed CR or PR according to RECIST v1.1 as assessed by the investigator, or meet the SD criteria at least once after start of study treatment at a minimum interval of 6 weeks. Subjects who do not have at least 1 post-baseline response assessment will be counted as non-responders.

Duration of Response (DOR) is defined as the time from the first documentation of objective response (CR or PR that is subsequently confirmed) to the first documentation of PD or death due to any cause, whichever comes first.

DOR data will be censored as described below:
Subjects who do not have PD and are still on study at the time of an analysis will be censored at the date of last disease assessment documenting absence of PD.
Subjects who started a new anticancer treatment prior to documentation of PD will be censored at the date of last disease assessment prior to the start of new treatment.
Subjects who are removed from the study prior to documentation of PD will be censored at the date of last disease assessment documenting absence of PD.

DOR will only be calculated for subjects who achieve a confirmed CR or PR.

Progression-free survival (PFS) is defined as the time from the start of study treatment to the first documentation of PD or death due to any cause, whichever comes first.

The same censoring rules as for DOR will be applied to PFS. Subjects lacking an evaluation of tumor response after their first dose of study drug will have their event time censored at Day 1.

Overall Survival (OS) is defined as the time from the start of study treatment to date of death due to any cause. In the absence of death, survival time will be censored at the last date the subject is known to be alive (i.e., date of last contact).

TABLE 8
Objectives and endpoints

| Primary Objective | Corresponding Primary Endpoint |
|---|---|
| Evaluate antitumor activity of LV | Investigator-determined confirmed ORR as |
| | measured by RECIST v1.1 |
| Secondary Objectives | Corresponding Secondary Endpoints |
| Evaluate the safety and tolerability of LV | Type, incidence, severity, seriousness, and |
| | relatedness of AEs |
| Evaluate stability and control of disease | Investigator-determined DCR as measured by |
| | RECIST v1.1 |
| Evaluate durability of response in subjects who | Investigator-determined DOR as measured by |
| respond to LV | RECIST v1.1 |
| Evaluate PFS of subjects treated with LV | Investigator-determined PFS as measured by |
| | RECIST v1.1 |
| Evaluate survival of subjects treated with LV | OS |
| Assess PK of LV | Selected PK parameters for LV, total antibody, |
| | and MMAE |
| Assess immunogenicity of LV | Incidence of ATAs to LV |
| Additional Objectives | Corresponding Additional Endpoints |
| Assess biomarkers of biological activity and | Relationship between biomarkers in blood and |
| resistance and predictive biomarkers of response | tumor tissue to efficacy, safety, or other |
| | biomarker endpoints following treatment with LV |

### Example 29: Anti-Tumor Activity of Ladiratuzumab Vedotin in Patients with Small Cell Lung Cancer

Human patients with unresectable, locally advanced or metastatic small cell lung cancer will be treated with ladiratuzumab vedotin (LV). Patients will have cancers that express LIV1. Patients will have extensive disease stage and will have disease progression during or following prior platinum-based systemic chemotherapy for extensive stage disease. Patients will have no more than 1 prior line of cytotoxic chemotherapy for the extensive disease stage. Patients may have received prior anti-PD(L)1 therapy, unless contraindicated. Patients will not have mixed SCLC/neuroendocrine tumors with NSCLC histologies.

LV will be administered by intravenous (IV) infusion at a dose of 1.0 mg/kg or 1.25 mg/kg on Day 1 of each 7-day cycle. Dosing may not exceed 100 mg or 125 mg per infusion for doses of 1.0 mg/kg and 1.25 mg/kg, respectively. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

### Example 30: Anti-Tumor Activity of Ladiratuzumab Vedotin in Patients with Squamous Non-Small Cell Lung Cancer

Human patients with unresectable, locally advanced or metastatic squamous non-small cell lung cancer will be treated with ladiratuzumab vedotin (LV). Patients will have cancers that express LIV1. Patients will have extensive disease stage and will have disease progression during or following prior platinum-based systemic chemotherapy for extensive stage disease. Patients will have no more than 1 prior line of cytotoxic chemotherapy for the extensive disease stage. Patients may have received prior anti-PD(L)1 therapy, unless contraindicated. Patients may have mixed histology NSCLC as long as the tumor is predominantly squamous histology. Patients will not have mixed SCLC/neuroendocrine tumors with NSCLC histologies. Patients will not have known epidermal growth factor receptor (EGFR), anaplastic lymphoma kinase (ALK), reactive oxygen species (ROS), BRAF, or other actionable mutations.

LV will be administered by intravenous (IV) infusion at a dose of 1.0 mg/kg or 1.25 mg/kg on Day 1 of each 7-day cycle. Dosing may not exceed 100 mg or 125 mg per infusion for doses of 1.0 mg/kg and 1.25 mg/kg, respectively. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

### Example 31: Anti-Tumor Activity of Ladiratuzumab Vedotin in Patients with Nonsquamous Non-Small Cell Lung Cancer

Human patients with unresectable, locally advanced or metastatic nonsquamous non-small cell lung cancer will be treated with ladiratuzumab vedotin (LV). Patients will have cancers that express LIV1. Patients will have disease progression during or following prior platinum-based systemic chemotherapy for extensive stage disease. Patients will have no more than 1 prior line of cytotoxic chemotherapy for their advanced disease. Patients may have received prior anti-PD(L)1 therapy, unless contraindicated. Patients will not have predominantly squamous cell histology NSCLC or small cell elements. Patients will not have known EGFR, ALK, ROS, BRAF, tropomyosin receptor kinase (TRK), or other actionable mutations.

LV will be administered by intravenous (IV) infusion at a dose of 1.0 mg/kg or 1.25 mg/kg on Day 1 of each 7-day cycle. Dosing may not exceed 100 mg or 125 mg per infusion for doses of 1.0 mg/kg and 1.25 mg/kg, respectively. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

### Example 32: Anti-Tumor Activity of Ladiratuzumab Vedotin in Patients with Head and Neck Squamous Cell Carcinoma

Human patients with unresectable, locally advanced or metastatic head and neck squamous cell carcinoma will be treated with ladiratuzumab vedotin (LV). Patients will have cancers that express LIV1. Patients will have squamous cell carcinoma of the head and neck with primary tumor site arising from the oral cavity, oropharynx, hypopharynx, or larynx. Patients will have extensive disease stage and will have disease progression during or following prior platinum-based systemic chemotherapy for extensive stage disease. Patients will have no more than 1 prior line of cytotoxic chemotherapy for the extensive disease stage. Patients may have received prior anti-PD(L)1 therapy, unless contraindicated.

LV will be administered by intravenous (IV) infusion at a dose of 1.0 mg/kg or 1.25 mg/kg on Day 1 of each 7-day cycle. Dosing may not exceed 100 mg or 125 mg per infusion for doses of 1.0 mg/kg and 1.25 mg/kg, respectively. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

### Example 33: Anti-Tumor Activity of Ladiratuzumab Vedotin in Patients with Esophageal Squamous Cell Carcinoma

Human patients with unresectable, locally advanced or metastatic esophageal squamous cell carcinoma will be treated with ladiratuzumab vedotin (LV). Patients will have cancers that express LIV1. Patients will have extensive disease stage and will have disease progression during or following prior systemic therapy. Patients will have had prior platinum-based chemotherapy. Patients will have no more than 1 prior line of cytotoxic chemotherapy for the extensive disease stage.

LV will be administered by intravenous (IV) infusion at a dose of 1.0 mg/kg or 1.25 mg/kg on Day 1 of each 7-day cycle. Dosing may not exceed 100 mg or 125 mg per infusion for doses of 1.0 mg/kg and 1.25 mg/kg, respectively. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

### Example 34: Anti-Tumor Activity of Ladiratuzumab Vedotin in Patients with Gastric or Gastroesophageal Adenocarcinoma

Human patients with unresectable, locally advanced or metastatic gastric or gastroesophageal adenocarcinoma will be treated with ladiratuzumab vedotin (LV). Patients will have cancers that express LIV1. Patients will have extensive disease stage and will have disease progression during or following prior systemic therapy. Patients will have had prior platinum-based chemotherapy. Patients will have no more than 1 prior line of cytotoxic chemotherapy for the extensive disease stage. Patients with human epidermal growth factor receptor 2 (HER2) will have received prior HER2-targeted therapy. Patients with deficient mismatch repair (dMMR) and/or microsatellite instability-high (MSI-H) may have received prior anti-PD(L)1 therapy, unless contraindicated.

LV will be administered by intravenous (IV) infusion at a dose of 1.0 mg/kg or 1.25 mg/kg on Day 1 of each 7-day cycle. Dosing may not exceed 100 mg or 125 mg per infusion for doses of 1.0 mg/kg and 1.25 mg/kg, respectively. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

1. A method of treating a subject having or at risk of having a LIV1-associated cancer, comprising:
administering to the subject a therapeutically effective dose of an antibody or an antigen-binding fragment thereof that specifically binds human LIV1,
wherein the antibody or antigen-binding fragment thereof comprises a heavy chain variable region (HCVR) having at least 95% identity to SEQ ID NO:1, and a light chain variable region (LCVR) having at least 95% identity to SEQ ID NO:2,
wherein the cancer is a solid tumor.

2. The method of claim 1, wherein the heavy chain variable region of the antibody or antigen-binding fragment thereof comprises the three complementarity determining regions (CDRs) of SEQ ID NO:1 and the light chain variable region of the antibody or antigen-binding fragment thereof comprises the three CDRs of SEQ ID NO:2.

3-4. (canceled)

5. The method of claim 1, wherein the heavy chain variable region comprises the sequence of SEQ ID NO:1 and the light chain variable region comprises the sequence of SEQ ID NO:2.

6. The method claim 1, wherein the antibody or antigen-binding fragment thereof is conjugated to monomethyl auristatin E (MMAE):

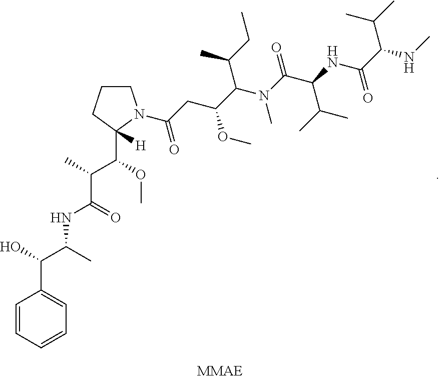 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

7. The method of claim 1, wherein the antibody or antigen-binding fragment thereof is conjugated to valine-citrulline-monomethyl auristatin E (vcMMAE):

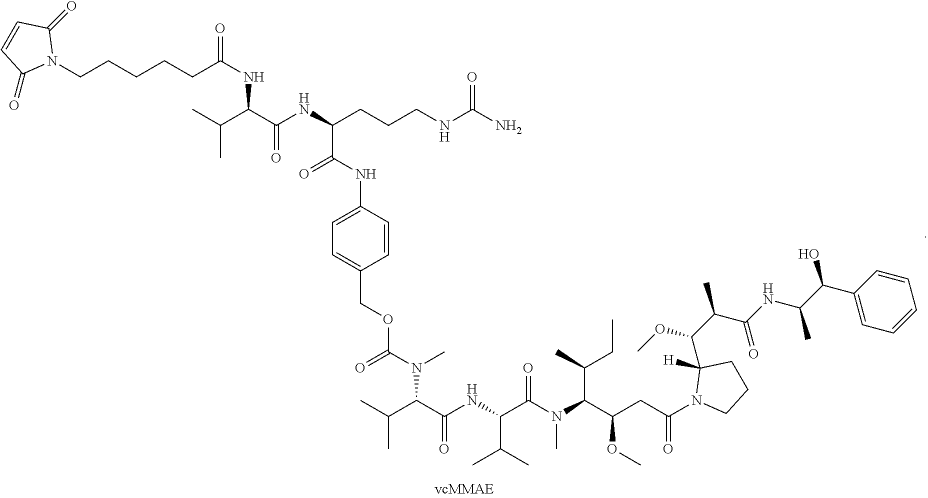 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

8-9. (canceled)

10. The method of claim 1, wherein the dose is about 2.5 mg/kg of body weight of the subject.

11-12. (canceled)

13. The method of claim 10, wherein the treatment cycle is a Q3W treatment cycle.

14. The method of claim 1, wherein the dose is about 1.0 mg/kg or about 1.25 mg/kg of body weight of the subject.

15-17. (canceled)

18. The method of claim 14, wherein the treatment cycle is a Q1W treatment cycle.

19-21. (canceled)

22. The method of claim 1, wherein the solid tumor is selected from the group consisting of lung cancer, head and neck cancer, esophageal cancer, gastric cancer, and gastroesophageal junction cancer.

23-74. (canceled)

75. The method of claim 1, wherein the cancer is an advanced stage cancer.

76-79. (canceled)

80. The method of claim 1, wherein the subject received prior treatment with standard of care therapy for the cancer and failed the prior treatment.

81. The method of claim 1, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express LIV1.

82. The method of claim 1, wherein one or more therapeutic effects in the subject is improved after administration of the antibody or antigen-binding fragment thereof relative to a baseline.

83. The method of claim 82, wherein the one or more therapeutic effects is selected from the group consisting of: size of a tumor derived from the cancer, objective response rate, duration of response, time to response, progression free survival, and overall survival.

84-92. (canceled)

93. The method of claim 1, wherein the route of administration for the antibody or antigen-binding fragment thereof is intravenous infusion.

94. The method claim 1, wherein the antibody or antigen-binding fragment thereof is administered as a monotherapy.

95. The method of claim 1, wherein the antibody or antigen-binding fragment thereof is administered in combination with a checkpoint inhibitor.

96-99. (canceled)

100. The method of claim 1, wherein the subject is a human.

101. A kit comprising:
(a) a dosage ranging from about 0.5 mg/kg to about 2.8 mg/kg of an antibody or antigen-binding fragment thereof that binds LIV1; and
(b) instructions for using the antibody or antigen-binding fragment thereof according to the method of claim 1.

102. A method of treating a subject having or at risk of having a LIV1-associated cancer, comprising:
administering to the subject a therapeutically effective dose of a LIV1 antibody-drug conjugate (LIV1-ADC), wherein the LIV1-ADC comprises a humanized hLIV22 antibody conjugated to a vcMMAE (valine-citruline-monomethyl auristatin E), wherein the hLIV22 antibody comprises a heavy chain variable region comprising the sequence of SEQ ID NO:1 and a light chain variable region comprising the sequence of SEQ ID NO:2,
wherein the vcMMAE has the structure: 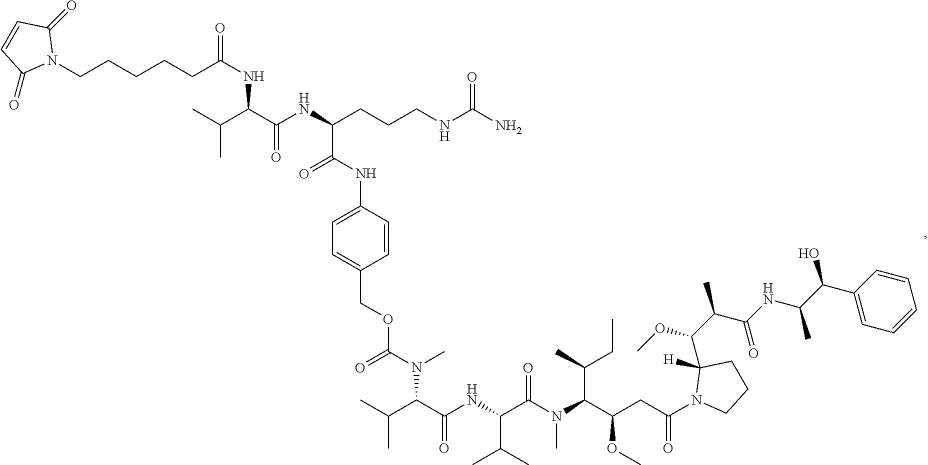 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
wherein the LIV1-ADC is administered about once per week.

103-105. (canceled)

106. The method of claim 102, wherein the LIV1-ADC is administered at a dose of about 1.0 mg/kg or about 1.25 mg/kg of body weight of the subject.

107-109. (canceled)

110. A method of treating a subject having or at risk of having a LIV1-associated cancer, comprising:
administering to the subject a therapeutically effective dose of a LIV1 antibody-drug conjugate (LIV1-ADC), wherein the LIV1-ADC comprises a humanized hLIV22 antibody conjugated to a vcMMAE (valine-citruline-monomethyl aurstating E), wherein the hLIV22 antibody comprises a heavy chain variable region comprising the sequence of SEQ ID NO:1 and a light chain variable region comprising the sequence of SEQ ID NO:2,
wherein the vcMMAE has the structure: 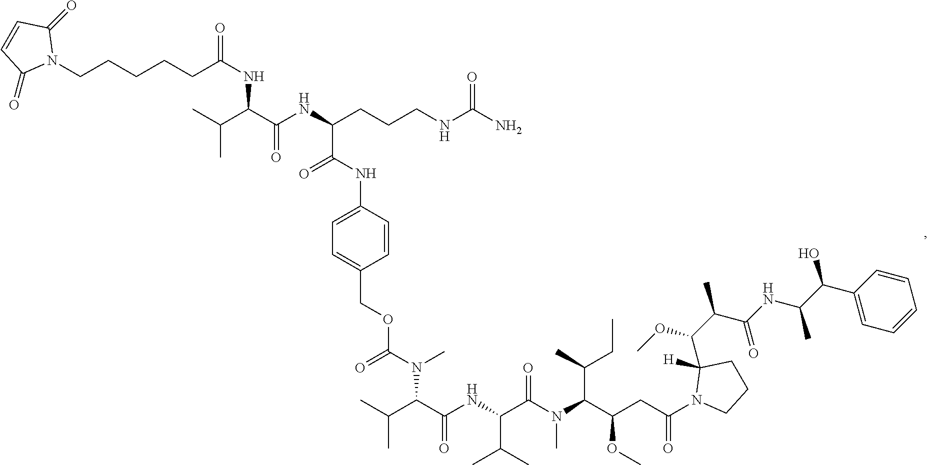 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
wherein the LIV1-ADC is administered twice in a three week treatment cycle.

111. The method of claim 110, wherein the LIV1-ADC is administered on day 1 and day 8 of the three week treatment cycle.

112. The method of claim 110, wherein the LIV1-ADC is administered is administered at a dose of about 0.5 mg/kg to about 3.0 mg/kg of body weight of the subject.

113-121. (canceled)

122. The method of claim 102, wherein the LIV1-associated cancer is a breast cancer.

123. The method of claim 122, wherein the breast cancer is selected from the group consisting of estrogen receptor positive (ER+) breast cancer, progesterone receptor positive/human epidermal growth factor receptor 2 negative (PR+/HER2−) breast cancer, triple negative breast cancer, hormone receptor positive (HR+) breast cancer, HER2 positive breast cancer, and HR+/HER2 negative breast cancer.

124-135. (canceled)

136. The method of claim 102, wherein the cancer is an advanced stage cancer.

137-141. (canceled)

142. The method of claim 102, wherein the subject received prior treatment with standard of care therapy for the cancer and failed the prior treatment.

143-145. (canceled)

146. The method of claim 102, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express LIV1.

147. The method of claim 102, wherein one or more therapeutic effects in the subject is improved after administration of the LIV1-ADC relative to a baseline.

148. The method of claim 147, wherein the one or more therapeutic effects is selected from the group consisting of: size of a tumor derived from the cancer, objective response rate, duration of response, time to response, progression free survival, and overall survival.

149-157. (canceled)

158. The method of claim 102, wherein the route of administration for the LIV1-ADC is intravenous infusion.

159. The method of claim 102, wherein the LIV1-ADC is administered as a monotherapy.

160. The method of claim 102, wherein the LIV1-ADC is administered in combination with trastuzumab.

161. (canceled)

162. The method of claim 102, wherein the subject is a human.

163. A kit comprising:
(a) a dosage ranging from about 0.5 mg/kg to about 3.0 mg/kg of a LIV1-ADC; and
(b) instructions for using the LIV1-ADC according to the method of claim 102.
